SEQUENCE LISTING (258 sequences)

SEQ ID NO: 1 - protein, 99 aa, Homo sapiens
VFVNTTESCEVERLFKATSPRGEPQDGLYCIRNSSTKSGKVLVVWDETSNKVRNYRIFEKDSKFYLEGEVLFVSVGSMVEHYHTHVLPSHQSLLLRHPY

SEQ ID NO: 2 - protein, 91 aa, Homo sapiens
WYHGPVSRNAAEYLLSSGINGSFLVRESESSPGQRSISLRYEGRVYHYRINTASDGKLYVSSESRFNTLAELVHHHSTVADGLITTLHYPA

SEQ ID NO: 3 - protein, 91 aa, Homo sapiens
WYHGPVSRSAAEYLLSSLINGSFLVRESESSPGQLSISLRYEGRVYHYRINTTADGKVYVTAESRFSTLAELVHHHSTVADGLVTTLHYPA

SEQ ID NO: 4 - protein, 100 aa, Homo sapiens
WYHGRIPRQVSENLVQRDGDFLVRDSLSSPGNFVLTCQWKNLAQHFKINRTVLRLSEAYSRVQYQFEMESFDSIPGLVRCYVGNRRPISQQSGAIIFQPI

SEQ ID NO: 5 - protein, 97 aa, Homo sapiens
WFFRSQGRKEAERQLLAPINKAGSFLIRESETNKGAFSLSVKDVTTQGELIKHYKIRCLDEGGYYISPRITFPSLQALVQHYSKKGDGLCQRLTLPC

SEQ ID NO: 6 - protein, 108 aa, Homo sapiens
WYAGACDRKSAEEALHRSNKDGSFLIRKSSGHDSKQPYTLVVFFNKRVYNIPVRFIEATKQYALGRKKNGEEYFGSVAEIIRNHQHSPLVLIDSQNNTKDSTRLKYAV

SEQ ID NO: 7 - protein, 97 aa, Homo sapiens
WFAGNISRSQSEQLLRQKGKEGAFMVRNSSQVGMYTVSLFSKAVNDKKGTVKHYHVHTNAENKLYLAENYCFDSIPKLIHYHQHNSAGMITRLRHPV

SEQ ID NO: 8 - protein, 97 aa, Homo sapiens
WYSKHMTRSQAEQLLKQEGKEGGFIVRDSSKAGKYTVSVFAKSTGDPQGVIRHYVVCSTPQSQYYLAEKHLFSTIPELINYHQHNSAGLISRLKYPV

SEQ ID NO: 9 - protein, 103 aa, Homo sapiens
QPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTG

SEQ ID NO: 10 - protein, 103 aa, Homo sapiens
QPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTG

SEQ ID NO: 11 - protein, 103 aa, Homo sapiens
QPWPTLLKNWQLLAVNHPGYMAFLTYDEVQERLQACRDKPGSYIFRPSCTRLGQWAIGYVSSDGSILQTIPANKPLSQVLLEGQKDGFYLYPDGKTHNPDLTE

SEQ ID NO: 12 - protein, 87 aa, Homo sapiens
EFHGMISREAADQLLIVAEGSYLIRESQRQPGTYTLALRFGSQTRNFRLYYDGKHFVGEKRFESIHDLVTDGLITLYIETKAAEYIA

SEQ ID NO: 13 - protein, 69 aa, Homo sapiens
EFHGIISREQADELLGGVEGAYILRESQRQPGCYTLALRFGNQTLNYRLFHDGKHFVGEKRFESIHDLV

SEQ ID NO: 14 - protein, 82 aa, Homo sapiens
WYWGSITASEARQHLQKMPEGTFLVRDSTHPSYLFTLSVKTTRGPTNVRIEYADSSFRLDSNCLSRPRILAFPDVVSLVQHY

SEQ ID NO: 15 - protein, 111 aa, Homo sapiens
WYIGEYSRQAVEEAFMKENKDGSFLVRDCSTKSKEEPYVLAVFYENKVYNVKIRFLERNQQFALGTGLRGDEKFDSVEDIIEHYKNFPIILIDGKDKTGVHRKQCHLTQPL

SEQ ID NO: 16 - protein, 106 aa, Homo sapiens
WYWGRLSRQEAVALLQGQRHGVFLVRDSSTSPGDYVLSVSENSRVSHYIINSSGPRPPVPPSPAQPPPGVSPSRLRIGDQEFDSLPALLEFYKIHYLDTTTLIEPV

SEQ ID NO: 17 - protein, 89 aa, Homo sapiens
WYMGPVSRQEAQTRLQGQRHGMFLVRDSSTCPGDYVLSVSENSRVSHYIINSLPNRRFKIGDQEFDHLPALLEFYKIHYLDTTTLIEPA

SEQ ID NO: 18 - protein, 90 aa, Homo sapiens
WFHGKITREQAERLLYPPETGLFLVRESTNYPGDYTLCVSCDGKVEHYRIMYHASKLSIDEEVYFENLMQLVEHYTSDADGLCTRLIKPK

SEQ ID NO: 19 - protein, 95 aa, Homo sapiens
WYHGNLTRHAAEALLLSNGCDGSYLLRDSNETTGLYSLSVRAKDSVKHFHVEYTGYSFKFGFNEFSSLKDFVKHFANQPLIGSETGTLMVLKHPY

SEQ ID NO: 20 - protein, 91 aa, Homo sapiens
WYHGAIPRIEAQELLKKQGDFLVRESHGKPGEYVLSVYSDGQRRHFIIQYVDNMYRFEGTGFSNIPQLIDHHYTTKQVITKKSGVVLLNPI

SEQ ID NO: 21 - protein, 90 aa, Homo sapiens
WYHGAIPRAEVAELLVHSGDFLVRESQGKQEYVLSVLWDGLPRHFIIQSLDNLYRLEGEGFPSIPLLIDHLLSTQQPLTKKSGVVLHRAV

SEQ ID NO: 22 - protein, 98 aa, Homo sapiens
WYFGKIGRKDAERQLLSPGNPQGAFLIRESETTKGAYSLSIRDWDQTRGDHVKHYKIRKLDMGGYYITTRVQFNSVQELVQHYMEVNDGLCNLLIAPC

SEQ ID NO: 23 - protein, 93 aa, Homo sapiens
WFFGAIGRSDAEKQLLYSENKTGSFLIRESESQKGEFSLSVLDGAVVKHYRIKRLDEGGFFLTRRRIFSTLNEFVSHYTKTSDGLCVKLGKPC

SEQ ID NO: 24 - protein, 98 aa, Homo sapiens
WYFGKLGRKDAERQLLSFGNPRGTFLIRESETTKGAYSLSIRDWDDMKGDHVKHYKIRKLDNGGYYITTRAQFETLQQLVQHYSERAAGLCCRLVVPC

SEQ ID NO: 25 - protein, 95 aa, Homo sapiens
WYSGRISRQLAEEILMKRNHLGAFLIRESESSPGEFSVSVNYGDQVQHFKVLREASGKYFLWEEKFNSLNELVDFYRTTTIAKKRQIFLRDEEPL

SEQ ID NO: 26 - protein, 92 aa, Homo sapiens
WFHEGLSRHQAENLLMGKEVGFFIIRASQSSPGDFSISVRHEDDVQHFKVMRDNKGNYFLWTEKFPSLNKLVDYYRTNSISRQKQIFLRDRT

SEQ ID NO: 27 - protein, 59 aa, Homo sapiens
WYSGRISRQLAEEILMKRNHLGAFLIRESESSPGEFSVSVNNRAQRGPCLGPKSHSRLG

SEQ ID NO: 28 - protein, 82 aa, Homo sapiens
WFHGRISREESHRIIKQQGLVDGLFLLRDSQSNPKAFVLTLCHHQKIKNFQILPCEDDGQTFFSLDDGNTKFSDLIQLVDFY

SEQ ID NO: 29 - protein, 97 aa, Homo sapiens
WFHHKISRDEAQRLIIQQGLVDGVFLVRDSQSNPKTFVLSMSHGQKIKHFQIIPVEDDGEMFHTLDDGHTRFTDLIQLVEFYQLNKGVLPCKLKHYC

SEQ ID NO: 30 - protein, 93 aa, Homo sapiens
WFFGKIPRAKAEEMLSKQRHDGAFLIRESESAPGDFSLSVKFGNDVQHFKVLRDGAGKYFLWVVKFNSLNELVDYHRSTSVSRNQQIFLRDIE

SEQ ID NO: 31 - protein, 97 aa, Homo sapiens
WFHGRISREESQRLIGQQGLVDGLFLVRESQRNPQGFVLSLCHLQKVKHYLILPSEEEGRLYFSMDDGQTRFTDLLQLVEFHQLNRGILPCLLRHCC

SEQ ID NO: 32 - protein, 98 aa, Homo sapiens
WFFKGISRKDAERQLLAPGNMLGSFMIRDSETTKGSYSLSVRDYDPRQGDTVKHYKIRTLDNGGFYISPRSTFSTLQELVDHYKKGNDGLCQKLSVPC

SEQ ID NO: 33 - protein, 92 aa, Homo sapiens
WFHGAISREDAENLLESQPLGSFLIRVSHSHVGYTLSYKAQSSCCHFMVKLLDDGTFMIPGEKVAHTSLDALVTFHQQKPIEPRRELLTQPC

SEQ ID NO: 34 - protein, 100 aa, Homo sapiens
WYNKSISRDKAEKLLLDTGKEGAFMVRDSRTAGTYTVSVFTKAVVSENNPCIKHYHIKETNDNPKRYYVAEKYVFDSIPLLINYHQHNGGGLVTRLRYPV

SEQ ID NO: 35 - protein, 106 aa, Homo sapiens
GCHGPICTEYAINKLRQEGSEEGMYVLRWSCTDFDNILMTVTCFEKSEQVQGAQKQFKNFQIEVQKGRYSLHGSDRSFPSLGDLMSHLKKQILRTDNISFMLKRCC

SEQ ID NO: 36 - protein, 82 aa, Homo sapiens
HGPISMDFAISKLKKAGNQTGLYVLRCSPKDFNKYFLTFAVERENVIEYKHCLITKNENEEYNLSGTKKNFSSLKDLLNCYQ

SEQ ID NO: 37 - protein, 101 aa, Homo sapiens
QCHGPITLDFAINKLKTGGSRPGSYVLRRSPQDFDSFLLTVCVQNPLGPDYKGCLIRRSPTGTFLLVGLSRPHSSLRELLATCWDGGLHVDGVAVTLTSCC

SEQ ID NO: 38 - protein, 93 aa, Homo sapiens
FFFGNITREEAEDYLVQGGMSDGLYLLRQSRNYLGGFALSVAHGRKAHHYTIERELNGTYAIAGGRTHASPADLCHYHSQESDGLVCLLKKPF

SEQ ID NO: 39 - protein, 92 aa, Homo sapiens
WFHGKISREESEQIVLIGSKTNGKFLIRARDNNGSYALCLLHEGKVLHYRIDKDKTGKLSIPEGKKFDTLWQLVEHYSYKADGLLRVLTVPC

SEQ ID NO: 40 - protein, 98 aa, Homo sapiens
WFFKNLSRKDAERQLLAPGNTHGSFLIRESESTAGSFSLSVRDFDQNQGEVVKHYKIRNLDNGGFYISPRITFPGLHELVRHYTNASDGLCTRLSRPC

SEQ ID NO: 41 - protein, 109 aa, Homo sapiens
WYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAA

SEQ ID NO: 42 - protein, 98 aa, Homo sapiens
WFFKDITRKDAERQLLAPGNSAGAFLIRESETLKGSFSLSVRDFDPVHGDVIKHYKIRSLDNGGYYISPRITFPCISDMIKHYQKQADGLCRRLEKAC

SEQ ID NO: 43 - protein, 90 aa, Homo sapiens
WFHGKISGQEAVQQLQPPEDGLFLVRESARHPGDYVLCVSFGRDVIHYRVLHRDGHLTIDEAVFFCNLMDMVEHYSKDKGAICTKLVRPK

SEQ ID NO: 44 - protein, 95 aa, Homo sapiens
WYYGKVTRHQAEMALNERGHEGDFLIRDSESSPNDFSVSLKAQGKNKHFKVQLKETVYCIGQRKFSTMEELVEHYKKAPIFTSEQGEKLYLVKHL

SEQ ID NO: 45 - protein, 95 aa, Homo sapiens
WYYGNVTRHQAECALNERGVEGDFLIRDSESSPSDFSVSLKASGKNKHFKVQLVDNVYCIGQRRFHTMDELVEHYKKAPIFTSEHGEKLYLVRAL

SEQ ID NO: 46 - protein, 95 aa, Homo sapiens
WFVEDINRVQAEDLLYGKPDGAFLIRESSKKGCYACSVVADGEVKHCVIYSTARGYGFAEPYNLYSSLKELVLHYQQTSLVQHNDSLNVRLAYPV

SEQ ID NO: 47 - protein, 96 aa, Homo sapiens
WYWGDISREEVNDKLRDMPDGTFLVRDASTKMQGDYTLTLRKGGNNKLIKIYHRDGKYGFSDPLTFNSVVELINHYHHESLAQYNPKLDVKLMYPV

SEQ ID NO: 48 - protein, 95 aa, Homo sapiens
WNVGSSNRNKAENLLRGKRDGTFLVRESSKQGCYACSVVVDGEVKHCVINKTATGYGFAEPYNLYSSLKELVLHYQHTSLVQHNDSLNVTLAYPV

SEQ ID NO: 49 - protein, 96 aa, Homo sapiens
WYWGDISREEVNEKLRDTADGTFLVRDASTKMHGDYTLTLRKGGNNKLIKIFHRDGKYGFSDPLTFSSVVELINHYRNESLAQYNPKLDVKLLYPV

SEQ ID NO: 50 - protein, 95 aa, Homo sapiens
WYVGKINRTQAEEMLSGKRDGTFLIRESSQRGCYACSVVVDGDTKHCVIYRTATGFGFAEPYNLYGSLKELVLHYQHASLVQHNDALTVTLAHPV

SEQ ID NO: 51 - protein, 96 aa, Homo sapiens
WYWGDISREEVNEKLRDTPDGTFLVRDASSKIQGEYTLTLRKGGNNKLIKVFHRDGHYGFSEPLTFCSVVDLINHYRHESLAQYNAKLDTRLLYPV

SEQ ID NO: 52 - protein, 89 aa, Homo sapiens
WYHASLTRAQAEHMLMRVPRDGAFLVRKRNEPNSYAISFRAEGKIKHCRVQQEGQTVMLGNSEFDSLVDLISYYEKHPLYRKMKLRYPI

SEQ ID NO: 53 - protein, 108 aa, Homo sapiens
WFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPV

SEQ ID NO: 54 - protein, 90 aa, Homo sapiens
WYYDSLSRGEAEDMLMRIPRDGAFLIRKREGSDSYAITFRARGKVKHCRINRDGRHFVLGTSAYFESLVELVSYYEKHSLYRKMRLRYPV

SEQ ID NO: 55 - protein, 104 aa, Homo sapiens
WFHKKVEKRTSAEKLLQEYCMETGGKDGTFLVRESETFPNDYTLSFWRSGRVQHCRIRSTMEGGTLKYYLTDNLTFSSIYALIQHYRETHLRCAEFELRLTDPV

SEQ ID NO: 56 - protein, 93 aa, Homo sapiens
WFFGCISRSEAVRRLQAEGNATGAFLIRVSEKPSADYVLSVRDTQAVRHYKIWRRAGGRLHLNEAVSFLSLPELVNYHRAQSLSHGLRLAAPC

SEQ ID NO: 57 - protein, 105 aa, Homo sapiens
WFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPL

SEQ ID NO: 58 - protein, 97 aa, Homo sapiens
WFHPNITGVEAENLLLTRGVDGSFLARPSKSNPGDFTLSVRRNGAVTHIKIQNTGDYYDLYGGEKFATLAELVQYYMEHHGQLKEKNGDVIELKYPL

SEQ ID NO: 59 - protein, 104 aa, Homo sapiens
WYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPY

SEQ ID NO: 60 - protein, 97 aa, Homo sapiens
WFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPL

SEQ ID NO: 61 - protein, 91 aa, Homo sapiens
WFHGKISKQEAYNLLMTVGQVCSFLVRPSDNTPGDYSLYFRTNENIQRFKICPTPNNQFMMGGRYYNSIGDIIDHYRKEQIVEGYYLKEPV

SEQ ID NO: 62 - protein, 92 aa, Homo sapiens
WYHGKLDRTIAEERLRQAGKSGSYLIRESDRRPGSFVLSFLSQMNVVNHFRIIAMCGDYYIGGRRFSSLSDLIGYYSHVSCLLKGEKLLYPV

SEQ ID NO: 63 - protein, 95 aa, Homo sapiens
WLQLQANAAAALHMLRTEPPGTFLVRKSNTRQCQALCMRLPEASGPSFVSSHYILESPGGVSLEGSELMFPDLVQLICAYCHTRDILLLPLQLPR

SEQ ID NO: 64 - protein, 94 aa, Homo sapiens
WLQLSLSEEEAAEVLQAQPPGIFLVHKSTKMQKKVLSLRLPCEFGAPLKEFAIKESTYTFSLEGSGISFADLFRLIAFYCISRDVLPFTLKLPY

SEQ ID NO: 65 - protein, 96 aa, Homo sapiens
WLQLSLGQAEVARILHRVVAGMFLVRRDSSSKQLVLCVHFPSLNESSAEVLEYTIKEEKSILYLEGSALVFEDIFRLIAFYCVSRDLLPFTLRLPQ

SEQ ID NO: 66 - protein, 99 aa, Homo sapiens
VYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEK

SEQ ID NO: 67 - protein, 97 aa, Homo sapiens
YYHGRLTKQDCETLLLKEGVDGNFLLRDSESIPGVLCLCVSFKNIVYTYRIFREKHGYYRIQTAEGSPKQVFPSLKELISKFEKPNQGMVVHLLKPI

SEQ ID NO: 68 - protein, 92 aa, Homo sapiens
WFHGFITRREAERLLEPKPQGCYLVRFSESAVTFVLTYRSRTCCRHFLLAQLRDGRHVVLGEDSAHARLQDLLLHYTAHPLSPYGETLTEPL

SEQ ID NO: 69 - protein, 101 aa, Homo sapiens
AWYHGLLSRQKAEALLQQNGDFLVRASGSRGGNPVISCRWRGSALHFEVFRVALRPRPGRPTALFQLEDEQFPSIPALVHSYMTGRRPLSQATGAVVSRPV

SEQ ID NO: 70 - protein, 94 aa, Homo sapiens
WFHGILTLKKANELLLSTGMPGSFLIRVSERIKGYALSYLSEDGCKHFLIDASADAYSFLGVDQLQHATLADLVEYHKEEPITSLGKELLLYPC

SEQ ID NO: 71 - protein, 93 aa, Homo sapiens
WFHGIISREDAEALLENMTEGAFLVRVSEKIWGYTLSYRLQKGFKHFLVDASGDFYSFLGVDPNRHATLTDLVDFHKEEIITVSGGELLQEPC

SEQ ID NO: 72 - protein, 99 aa, Homo sapiens
WFHGMLSRLKAAQLVLTGGTGSHGVFLVRQSETRRGEYVLTFNFQGKAKHLRLSLNEEGQCRVQHLWFQSIFDMLEHFRVHPIPLESGGSSDVVLVSYV

SEQ ID NO: 73 - protein, 99 aa, Homo sapiens
WFHGTLSRVKAAQLVLAGGPRNHGLFVIRQSETRPGEYVLTFNFQGKAKHLRLSLNGHGQCHVQHLWFQSVLDMLRHFHTHPIPLESGGSADITLRSYV

SEQ ID NO: 74 - protein, 99 aa, Homo sapiens
WFHGPISRVKAAQLVQLQGPDAHGVFLVRQSETRRGEYVLTFNFQGIAKHLRLSLTERGQCRVQHLHFPSVVDMLHHFQRSPIPLECGAACDVRLSSYV

SEQ ID NO: 75 - protein, 100 aa, Homo sapiens
WYHGRIPREVSETLVQRNGDFLIRDSLTSLGDYVLTCRWRNQALHFKINKVVVKAGESYTHIQYLFEQESFDHVPALVRYHVGSRKAVSEQSGAIIYCPV

SEQ ID NO: 76 - protein, 97 aa, Homo sapiens
WAFAGISRPCALALLRRDVLGAFLLWPELGASGQWCLSVRTQCGVVPHQVFRNHLGRYCLEHLPAEFPSLEALVENHAVTERSLFCPLDMGRLNPTY

SEQ ID NO: 77 - protein, 109 aa, Homo sapiens
WYSGNCDRYAVESALLHLQKDGAYTVRPSSGPHGSQPFTLAVLLRGRVFNIPIRRLDGGRHYALGREGRNREELFSSVAAMVQHFMWHPLPLVDRHSGSRELTCLLFPT

SEQ ID NO: 78 - protein, 92 aa, Homo sapiens
WFHGFITRKQTEQLLRDKALGSFLIRLSDRATGYILSYRGSDRCRHFVINQLRNRRYIISGDTQSHSTLAELVHHYQEAQLEPFKEMLTAAC

SEQ ID NO: 79 - protein, 95 aa, Homo sapiens
WYHGAISRGDAENLLRLCKECSYLVRNSQTSKHDYSLSLRSNQGFMHMKLAKTKEKYVLGQNSPPFDSVPEVIHYYTTRKLPIKGAEHLSLLYPV

SEQ ID NO: 80 - protein, 92 aa, Homo sapiens
WFHGKLSRREAEALLQLNGDFLVRESTTTPGQYVLTGLQSGQPKHLLLVDPEGVVRTKDHRFESVSHLISYHMDNHLPIISAGSELCLQQPV

SEQ ID NO: 81 - protein, 92 aa, Homo sapiens
WYHGRMSRRAAERMLRADGDFLVRDSVTNPGQYVLTGMHAGQPKHLLLVDPEGVVRTKDVLFESISHLIDHHLQNGQPIVAAESELHLRGVV

SEQ ID NO: 82 - protein, 92 aa, Homo sapiens
WYQGEMSRKEAEGLLEKDGDFLVRKSTTNPGSFVLTGMHNGQAKHLLLVDPEGTIRTKDRVFDSISHLINHHLESSLPIVSAGSELCLQQPV

SEQ ID NO: 83 - protein, 92 aa, Homo sapiens
CYHGKLSRKAAESLLVKDGDFLVRESATSPGQYVLSGLQGGQAKHLLLVDPEGKVRTKDHVFDNVGHLIRYHMDNSLPIISSGSEVSLKQPV

SEQ ID NO: 84 - protein, 96 aa, Homo sapiens
WFHGPLNRADAESLLSLCKEGSYLVRLSETNPQDCSLSLRSSQGFLHLKFARTRENQVVLGQHSGPFPSVPELVLHYSSRPLPVQGAEHLALLYPV

SEQ ID NO: 85 - protein, 96 aa, Homo sapiens
WYHGAISRAEAESRLQPCKEAGYLVRNSESGNSRYSIALKTSQGCVHIIVAQTKDNKYTLNQTSAVFDSIPEVVHYYSNEKLPFKGAEHMTLLYPV

SEQ ID NO: 86 - protein, 96 aa, Homo sapiens
WYHGAISRTDAENLLRLCKEASYLVRNSETSKNDFSLSLKSSQGFMHMKLSRTKEHKYVLGQNSPPFSSVPEIVHHYASRKLPIKGAEHMSLLYPV

SEQ ID NO: 87 - protein, 97 aa, Homo sapiens
WNHGNITRSKAEELLSRTGKDGSFLVRASESISRAYALCVLYRNCVYTYRILPNEDDKFTVQASEGVSMRFFTKLDQLIEFYKKENMGLVTHLQYPV

SEQ ID NO: 88 - protein, 97 aa, Homo sapiens
WYHRDLSRAAAEELLARAGRDGSFLVRDSESVAGAFALCVLYQKHVHTYRILPDGEDFLAVQTSQGVPVRRFQTLGELIGLYAQPNQGLVCALLLPV

SEQ ID NO: 89 - protein, 92 aa, Homo sapiens
WLFEGLGRDKAEELLQLPDTKVGSFMIRESETKKGFYSLSVRHRQVKHYRIFRLPNNWYYISPRLTFQCLEDLVNHYSEVADGLCCVLTTPC

SEQ ID NO: 90 - protein, 98 aa, Homo sapiens
WLYEGLSREKAEELLLLPGNPGGAFLIRESQTRRGSYSLSVRLSRPASWDRIRHYRIHCLDNGWLYISPRLTFPSLQALVDHYSELADDICCLLKEPC

SEQ ID NO: 91 - protein, 95 aa, Homo sapiens
FYWGPLSVHGAHERLRAEPVGTFLVRDSRQRNCFFALSVKMASGPTSIRVHFQAGRFHLDGSRESFDCLFELLEHYVAAPRRMLGAPLRQRRVRP

SEQ ID NO: 92 - protein, 109 aa, Homo sapiens
WYWGSMTVNEAKEKLKEAPEGTFLIRDSSHSDYLLTISVKTSAGPTNLRIEYQDGKFRLDSIICVKSKLKQFDSVVHLIDYYVQMCKDKRTGPEAPRNGTVHLYLTKPL

SEQ ID NO: 93 - protein, 97 aa, Homo sapiens
FYWSAVTGGEANLLLSAEPAGTFLIRDSSDQRHFFTLSVKTQSGTKNLRIQCEGGSFSLQSDPRSTQPVPRFDCVLKLVHHYMPPPGAPSFPSPPTE

SEQ ID NO: 94 - protein, 96 aa, Homo sapiens
CYWGVMDKYAAEALLEGKPEGTFLLRDSAQEDYLFSVSFRRYSRSLHARIEQWNHNFSFDAHDPCVFHSPDITGLLEHYKDPSACMFFEPLLSTPL

SEQ ID NO: 95 - protein, 96 aa, Homo sapiens
CYWGVMDRYEAEALLEGKPEGTFLLRDSAQEDYLFSVSFRRYNRSLHARIEQWNHNFSFDAHDPCVFHSSTVTGLLEHYKDPSSCMFFEPLLTISL

SEQ ID NO: 96 - protein, 97 aa, Homo sapiens
WYWGPITRWEAEGKLANVPDGSFLVRDSSDDRYLLSLSFRSHGKTLHTRIEHSNGRFSFYEQPDVEGHTSIVDLIEHSIRDSENGAFCYSRSRLPGS

SEQ ID NO: 97 - protein, 99 aa, Homo sapiens
WYWGPMNWEDAEMKLKGKPDGSFLVRDSSDPRYILSLSFRSQGITHHTRMEHYRGTFSLWCHPKFEDRCQSVVEFIKRAIMHSKNGKFLYFLRSRVPGL

SEQ ID NO: 98 - protein, 107 aa, Homo sapiens
YIKRVIAHPSFHNINFKQAEKMMETMDQGDVIIRPSSKGENHLTVTWKVSDGIYQHVDVREEGKENAFSLGATLWINSEEFEDLDEIVARYVQPMASFARDLLNHKY

SEQ ID NO: 99 - protein, 98 aa, Homo sapiens
WYFGKITRRESERLLLNAENPRGTFLVRESETTKGAYCLSVSDFDNAKGLNVKHYKIRKLDSGGFYITSRTQFNSLQQLVAYYSKHADGLCHRLTTVC

SEQ ID NO: 100 - protein, 93 aa, Homo sapiens
WYFSGVSRTQAQQLLLSPPNEPGAFLIRPSESSLGGYSLSVRAQAKVCHYRVSMAADGSLYLQKGRLFPGLEELLTYYKANWKLIQNPLLQPC

SEQ ID NO: 101 - protein, 104 aa, Homo sapiens
ACFYTVSRKEATEMLQKNPSLGNMILRPGSDSRNYSITIRQEIDIPRIKHYKVMSVGQNYTIELEKPVTLPNLFSVIDYFVKETRGNLRPFICSTDENTGQEPS

SEQ ID NO: 102 - protein, 116 aa, Homo sapiens
YMMSEVLAKEEARRALETPSCFLKVSRLEAQLLLERYPECGNLLLRPSGDGADGVSVTTRQMHNGTHVVRHYKVKREGPKYVIDVEQPFSCTSLDAVVNYFVSHTKKALVPFLLDE

SEQ ID NO: 103 - protein, 98 aa, Homo sapiens
WNDGCIMGFISKERERALLKDQQPGTFLLRFSESSREGAITFTWVERSQNGGEPDFHAVEPYTKKELSAVTFPDIIRNYKVMAAENIPENPLKYLYPN

SEQ ID NO: 104 - protein, 96 aa, Homo sapiens
WNDGRIMGFVSRSQERRLLKKTMSGTFLLRFSESSEGGITCSWVEHQDDDKVLIYSVQPYTKEVLQSLPLTEIIRHYQLLTEENIPENPLRFLYPR

SEQ ID NO: 105 - protein, 91 aa, Homo sapiens
WNEGYIMGFISKERERAILSTKPPGTFLLRFSESSKEGGVTFTWVEKDISGKTQIQSVEPYTKQQLNNMSFAEIIMGYKIMDATNILVSPL

SEQ ID NO: 106 - protein, 96 aa, Homo sapiens
WIDGYVMGFVSKEKERLLLKDKMPGTFLLRFSESHLGGITFTWVDHSESGEVRFHSVEPYNKGRLSALPFADILRDYKVIMAENIPENPLKYLYPD

SEQ ID NO: 107 - protein, 98 aa, Homo sapiens
WNDGAILGFVNKQQAHDLLINKPDGTFLLRFSDSEIGGITIAWKFDSPERNLWNLKPFTTRDFSIRSLADRLGDLSYLIYVFPDRPKDEVFSKYYTPV

SEQ ID NO: 108 - protein, 98 aa, Homo sapiens
WNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPV

SEQ ID NO: 109 - protein, 116 aa, Homo sapiens
WFDGVLDLTKRCLRSYWSDRLIIGFISKQYVTSLLLNEPDGTFLLRFSDSEIGGITIAHVIRGQDGSPQIENIQPFSAKDLSIRSLGDRIRDLAQLKNLYPKKPKDEAFRSHYKPE

SEQ ID NO: 110 - protein, 99 aa, Homo sapiens
WYCRNMNRSKAEQLLRSEDKEGGFMVRDSSQPGLYTVSLYTKFGGEGSSGFRHYHIKETTTSPKKYYLAEKHAFGSIPEIIEYHKHNAAGLVTRLRYPV

SEQ ID NO: 111 - protein, 110 aa, Homo sapiens
WYKPEISREQAIALLKDQEPGAFIIRDSHSFRGAYGLAMKVSSPPPTIMQQNKKGDMTHELVRHFLIETGPRGVKLKGCPNEPNFGSLSALVYQHSIIPLALPCKLVIPN

SEQ ID NO: 112 - protein, 111 aa, Homo sapiens
WYKADISREQAIAMLKDKEPGSFIVRDSHSFRGAYGLAMKVATPPPSVLQLNKKAGDLANELVRHFLIECTPKGVRLKGCSNEPYFGSLTALVCQHSITPLALPCKLLIPE

SEQ ID NO: 113 - protein, 108 aa, Homo sapiens
WFKPNITREQAIELLRKEEPGAFVIRDSSSYRGSFGLALKVQEVPASAQSRPGEDSNDLIRHFLIESSAKGVHLKGADEEPYFGSLSAFVCQHSIMALALPCKLTIPQ

SEQ ID NO: 114 - protein, 108 aa, Homo sapiens
WYKPHLSRDQAIALLKDKDPGAFLIRDSHSFQGAYGLALKVATPPPSAQPWKGDPVEQLVRHFLIETGPKGVKIKGCPSEPYFGSLSALVSQHSISPISLPCCLRIPS

SEQ ID NO: 115 - protein, 97 aa, Homo sapiens
WYHRNITRNQAEHLLRQESKEGAFIVRDSRHLGSYTISVFMGARRSTEAAIKHYQIKKNDSGQWYVAERHAFQSIPELIWYHQHNAAGLMTRLRYPV

SEQ ID NO: 116 - protein, 80 aa, Homo sapiens
GIHGPLLEPFVQAKLRPEDGLYLIHWSTSHPYRLILTVAQRSQAPDGMQSLRLRKFPIEQQDGAFVLEGWGRSFPSVREL

SEQ ID NO: 117 - protein, 95 aa, Homo sapiens
WYAGPMERAGAESILANRSDGTFLVRQRVKDAAEFAISIKYNVEVKHIKIMTAEGLYRITEKKAFRGLTELVEFYQQNSLKDCFKSLDTTLQFPF

SEQ ID NO: 118 - protein, 95 aa, Homo sapiens
WFAGNMERQQTDNLLKSHASGTYLIRERPAEAERFAISIKFNDEVKHIKVVEKDNWIHITEAKKFDSLLELVEYYQCHSLKESFKQLDTTLKYPY

SEQ ID NO: 119 - protein, 95 aa, Homo sapiens
WYAGAMERLQAETELINRVNSTYLVRHRTKESGEYAISIKYNNEAKHIKILTRDGFFHIAENRKFKSLMELVEYYKHHSLKEGFRTLDTTLQFPY

SEQ ID NO: 120 - protein, 98 aa, Homo sapiens
WYFGKMGRKDAERLLLNPGNQRGIFLVRESETTKGAYSLSIRDWDEIRGDNVKHYKIRKLDNGGYYITTRAQFDTLQKLVKHYTEHADGLCHKLTTVC

SEQ ID NO: 121 - protein, 92 aa, Homo sapiens
WYHSSLTREEAERKLYSGAQTDGKFLLRPRKEQGTYALSLIYGKTVYHYLISQDKAGKYCIPEGTKFDTLWQLVEYLKLKADGLIYCLKEAC

SEQ ID NO: 122 - protein, 93 aa, Homo sapiens
FFYGSISRAEAEEHLKLAGMADGLFLLRQCLRSLGGYVLSLVHDVRFHHFPIERQLNGTYAIAGGKAHCGPAELCEFYSRDPDGLPCNLRKPC

SEQ ID NO: 123 - protein, 6 aa, synthetic construct
GQSQPD

SEQ ID NO: 124 - protein, 6 aa, synthetic construct
RQRKQE

SEQ ID NO: 125 - protein, 6 aa, synthetic construct
SPRIQE

SEQ ID NO: 126 - protein, 6 aa, synthetic construct
SQGRKV

SEQ ID NO: 127 - protein, 6 aa, synthetic construct
SISKQG

SEQ ID NO: 128 - protein, 6 aa, synthetic construct
SQTYPG

SEQ ID NO: 129 - protein, 7 aa, synthetic construct
SETVKGA

SEQ ID NO: 130 - protein, 160 aa, synthetic construct
MKIEEHHHHHHSSGKLGLNDIFEAQKIEWHESSGEDLYFQSGTTMDDYKDDDDKGGSEVQKPLHEQLWYHGAIPRAEVAELLVHSGDFLVREGQSQPDYVLSVLWDGLPRHFIIQSLDNLYRLEGEGFPSIPLLIDHLLSTQQPLTKKSGVVLHRAVPSR

SEQ ID NO: 131 - protein, 160 aa, synthetic construct
MKIEEHHHHHHSSGKLGLNDIFEAQKIEWHESSGEDLYFQSGTTMDDYKDDDDKGGSEVQKPLHEQLWYHGAIPRAEVAELLVHSGDFLVRERQRKQEYVLSVLWDGLPRHFIIQSLDNLYRLEGEGFPSIPLLIDHLLSTQQPLTKKSGVVLHRAVPSR

SEQ ID NO: 132 - protein, 160 aa, synthetic construct
MKIEEHHHHHHSSGKLGLNDIFEAQKIEWHESSGEDLYFQSGTTMDDYKDDDDKGGSEVQKPLHEQLWYHGAIPRAEVAELLVHSGDFLVRESPRIQEYVLSVLWDGLPRHFIIQSLDNLYRLEGEGFPSIPLLIDHLLSTQQPLTKKSGVVLHRAVPSR

SEQ ID NO: 133 - protein, 160 aa, synthetic construct
MKIEEHHHHHHSSGKLGLNDIFEAQKIEWHESSGEDLYFQSGTTMDDYKDDDDKGGSEVQKPLHEQLWYHGAIPRAEVAELLVHSGDFLVRESQGRKVYVLSVLWDGLPRHFIIQSLDNLYRLEGEGFPSIPLLIDHLLSTQQPLTKKSGVVLHRAVPSR

SEQ ID NO: 134 - protein, 160 aa, synthetic construct
MKIEEHHHHHHSSGKLGLNDIFEAQKIEWHESSGEDLYFQSGTTMDDYKDDDDKGGSEVQKPLHEQLWYHGAIPRAEVAELLVHSGDFLVRESISKQGYVLSVLWDGLPRHFLIQSLDNLYRLEGEGFPSIPLLIDHLLSTQQPLTKKSGVVLHRAVPSR

SEQ ID NO: 135 - protein, 160 aa, synthetic construct
MKIEEHHHHHHSSGKLGLNDIFEAQKIEWHESSGEDLYFQSGTTMDDYKDDDDKGGSEVQKPLHEQLWYHGAIPRAEVAELLVHSGDFIVRESQTYPGYVLSVLWDGLPRHFIIQSLDNLYRLEGEGFPSIPLLIDHLLSTQQPLTKKSGVVLHRAVPSR

SEQ ID NO: 136 - protein, 165 aa, synthetic construct
MKIEEHHHHHHSSGKLGLNDIFEAQKIEWHESSGEDLYFQSGTTMDDYKDDDDKGGSDSIQAEEWYFGKITRRESERLLLNAENPRGTFLVRESETVKGAYALSVSDFDNAKGLNVKHYLIRKLDSGGFYITSRTQFNSLQQLVAYYSKHADGLCHRLTTVCPSR

SEQ ID NO: 137 - protein, 180 aa, synthetic construct
MKIEEHHHHHHSSGKLGLNDIFEAQKIEWHESSGEDLYFQSGTTMDDYKDDDDKGGSSPSNYITPVNSLEKHSWYHGPVSRNAAEYLLSSGINGSFLVREGQSQPDRVISLRYEGRVYHYIINTASDGKLYVSSESRFNTLAELVHHHSTVADGLITTLHYPAPKRNKPTVYGVSPNYSR

SEQ ID NO: 138 - protein, 181 aa, synthetic construct
MKIEEHHHHHHSSGKLGLNDIFEAQKIEWHESSGEDLYFQSGTTMDDYKDDDDKGGSSPSNYITPVNSLEKHSWYHGPVSRNAAEYLLSSGINGSFLVRESETVKGARAISLRYEGRVYHYLINTASDGKLYVSSESRFNTLAELVHHHSTVADGLITTLHYPAPKRNKPTVYGVSPNYSR

SEQ ID NO: 139 - protein, 173 aa, synthetic construct
MKIEEHHHHHHSSGKLGLNDIFEAQKIEWHESSGEDLYFQSGTTMDDYKDDDDKGSARVESLEMERWFFRSQGRKEAERQLLAPINKAGSFLIREGQSQPDFVLSVKDVTTQGELIKHYIIRCLDEGGYYISPRITFPSLQALVQHYSKKGDGLCQRLTLPCVRPAPQNPWSR

SEQ ID NO: 140 - protein, 174 aa, synthetic construct
MKIEEHHHHHHSSGKLGLNDIFEAQKIEWHESSGEDLYFQSGTTMDDYKDDDDKGSARVESLEMERWFFRSQGRKEAERQLLAPINKAGSFLIRESETVKGAFALSVKDVTTQGELIKHYLIRCLDEGGYYISPRITFPSLQALVQHYSKKGDGLCQRLTLPCVRPAPQNPWSR

SEQ ID NO: 141 - protein, 174 aa, synthetic construct
MKIEEHHHHHHSSGKLGLNDIFEAQKIEWHESSGEDLYFQSGTTMDDYKDDDDKGSEAEDSIEMYEWYSKHMTRSQAEQLLKQEGKEGGFIVRDGQSQPDYVVSVFAKSTGDPQGVIRHYIVCSTPQSQYYLAEKHLFSTIPELINYHQHNSAGLISRLKYPVSQQNKNAPSSR

SEQ ID NO: 142 - protein, 175 aa, synthetic construct
MKIEEHHHHHHSSGKLGLNDIFEAQKIEWHESSGEDLYFQSGTTMDDYKDDDDKGSEAEDSIEMYEWYSKHMTRSQAEQLLKQEGKEGGFIVRDSETVKGAYAVSVFAKSTGDPQGVIRHYLVCSTPQSQYYLAEKHLFSTIPELINYHQHNSAGLISRLKYPVSQQNKNAPSSR

SEQ ID NO: 143 - protein, 165 aa, synthetic construct
MKIEEHHHHHHSSGKLGLNDIFEAQKIEWHESSGEDLYFQSGTTMDDYKDDDDKGSARFDSSDRSAWYMGPVSRQEAQTRLQGQRHGMFLVRDGQSQPDYVLSVSENSRVSHYIINSLPNRRFKIGDQEFDHLPALLEFYKIHYLDTTTLIEPAPRYPSPPMGSR

SEQ ID NO: 144 - protein, 166 aa, synthetic construct
MKIEEHHHHHHSSGKLGLNDIFEAQKIEWHESSGEDLYFQSGTTMDDYKDDDDKGSARFDSSDRSAWYMGPVSRQEAQTRLQGQRHGMFLVRDSETVKGAYALSVSENSRVSHYLINSLPNRRFKIGDQEFDHLPALLEFYKIHYLDTTTLIEPAPRYPSPPMGSR

SEQ ID NO: 145 - protein, 161 aa, synthetic construct
MKIEEHHHHHHSSGKLGLNDIFEAQKIEWHESSGEDLYFQSGTTMDDYKDDDDKGGSEVQKPLHEQLWYHGAIPRAEVAELLVHSGDFLVRESETVKGAYALSVLWDGLPRHFLIQSLDNLYRLEGEGFPSIPLLIDHLLSTQQPLTKKSGVVLHRAVPSR

SEQ ID NO: 146 - protein, 173 aa, synthetic construct
MKIEEHHHHHHSSGKLGLNDIFEAQKIEWHESSGEDLYFQSGTTMDDYKDDDDKGGSPKNYIEMKPHPWFFGKIPRAKAEEMLSKQRHDGAFLIREGQSQPDFVLSVKFGNDVQHFIVLRDGAGKYFLWVVKFNSLNELVDYHRSTSVSRNQQIFLRDIEQVPQQPTYVQASR

SEQ ID NO: 147 - protein, 189 aa, synthetic construct
MKIEEHHHHHHSSGKLGLNDIFEAQKIEWHESSGEDLYFQSGTTMDDYKDDDDKGGSPKNYIEMKPHPWFFGKIPRAKAEEMLSKQRHDGAFLIRESETVKGAFALSVKFGNDVQHFLVLRDGAGKYFLWVVKFNSLNELVDYHRSTSVSRNQQIFLRDIEQVPQQPTYVQASRYNPVILIMKRWHRYN

SEQ ID NO: 148 - protein, 174 aa, synthetic construct
MKIEEHHHHHHSSGKLGLNDIFEAQKIEWHESSGEDLYFQSGTTMDDYKDDDDKGSAKANSLEPEPWFFKNLSRKDAERQLLAPGNTHGSFLIREGQSQPDFVLSVRDFDQNQGEVVKHYIIRNLDNGGFYISPRITFPGLHELVRHYTNASDGLCTRLSRPCQTQKPQKPWSR

SEQ ID NO: 149 - protein, 175 aa, synthetic construct
MKIEEHHHHHHSSGKLGLNDIFEAQKIEWHESSGEDLYFQSGTTMDDYKDDDDKGSAKANSLEPEPWFFKNLSRKDAERQLLAPGNTHGSFLIRESETVKGAFALSVRDFDQNQGEVVKHYLIRNLDNGGFYISPRITFPGLHELVRHYTNASDGLCTRLSRPCQTQKPQKPWSR

SEQ ID NO: 150 - protein, 163 aa, synthetic construct
MKIEEHHHHHHSSGKLGLNDIFEAQKIEWHESSGEDLYFQSGTTMDDYKDDDDKGSSLTGKFAGNPWYYGKVTRHQAEMALNERGHEGDFLIRDGQSQPDFVVSLKAQGKNKHFIVQLKETVYCIGQRKFSTMEELVEHYKKAPIFTSEQGEKLYLVKHLSSR

SEQ ID NO: 151 - protein, 164 aa, synthetic construct
MKIEEHHHHHHSSGKLGLNDIFEAQKIEWHESSGEDLYFQSGTTMDDYKDDDDKGSSLTGKFAGNPWYYGKVTRHQAEMALNERGHEGDFLIRDSETVKGAFAVSLKAQGKNKHFLVQLKETVYCIGQRKFSTMEELVEHYKKAPIFTSEQGEKLYLVKHLSSR

SEQ ID NO: 152 - protein, 171 aa, synthetic construct
MKIEEHHHHHHSSGKLGLNDIFEAQKIEWHESSGEDLYFQSGTTMDDYKDDDDKGSDSSVSLQDAEWYWGDISREEVNDKLRDMPDGTFLVRDGQSQPDYVLTLRKGGNNKLIIIYHRDGKYGFSDPLTFNSVVELINHYHHESLAQYNPKLDVKLMYPVSRYQQDQLVSR

SEQ ID NO: 153 - protein, 172 aa, synthetic construct
MKIEEHHHHHHSSGKLGLNDIFEAQKIEWHESSGEDLYFQSGTTMDDYKDDDDKGSDSSVSLQDAEWYWGDISREEVNDKLRDMPDGTFLVRDSETVKGAYALTLRKGGNNKLILIYHRDGKYGFSDPLTFNSVVELINHYHHESLAQYNPKLDVKLMYPVSRYQQDQLVSR

SEQ ID NO: 154 - protein, 161 aa, synthetic construct
MKIEEHHHHHHSSGKLGLNDIFEAQKIEWHESSGEDLYFQSGTTMDDYKDDDDKGGSSLQNAEWYWGDISREEVNEKLRDTADGTFLVRDGQSQPDYVLTLRKGGNNKLIIIFHRDGKYGFSDPLTFSSVVELINHYRNESLAQYNPKLDVKLLYPVSKSR

SEQ ID NO: 155 - protein, 162 aa, synthetic construct
MKIEEHHHHHHSSGKLGLNDIFEAQKIEWHESSGEDLYFQSGTTMDDYKDDDDKGGSSLQNAEWYWGDISREEVNEKLRDTADGTFLVRDSETVKGAYALTLRKGGNNKLILIFHRDGKYGFSDPLTFSSVVELINHYRNESLAQYNPKLDVKLLYPVSKSR

SEQ ID NO: 156 - protein, 171 aa, synthetic construct
MKIEEHHHHHHSSGKLGLNDIFEAQKIEWHESSGEDLYFQSGTTMDDYKDDDDKGSGSPPSLQDAEWYWGDISREEVNEKLRDTPDGTFLVRDGQSQPDYVLTLRKGGNNKLIIVFHRDGHYGFSEPLTFCSVVDLINHYRHESLAQYNAKLDTRLLYPVSRYQQDQIVSR

SEQ ID NO: 157 - protein, 172 aa, synthetic construct
MKIEEHHHHHHSSGKLGLNDIFEAQKIEWHESSGEDLYFQSGTTMDDYKDDDDKGSGSPPSLQDAEWYWGDISREEVNEKLRDTPDGTFLVRDSETVKGAYALTLRKGGNNKLILVFHRDGHYGFSEPLTFCSVVDLINHYRHESLAQYNAKLDTRLLYPVSKYQQDQIVSR

SEQ ID NO: 158 - protein, 181 aa, synthetic construct
MKIEEHHHHHHSSGKLGLNDIFEAQKIEWHESSGEDLYFQSGTTMDDYKDDDDKGSLNCADPTSERWFHGHLSGKEAEKLLTEKGKHGSFLVREGQSQPDFVLSVRTGDDKGESNDGKSKVTHVIIRCQELKYDVGGGERFDSLTDLVEHYKKNPMVETLGTVLQLKQPLNTTRINAADSR

SEQ ID NO: 159 - protein, 182 aa, synthetic construct
MKIEEHHHHHHSSGKLGLNDIFEAQKIEWHESSGEDLYFQSGTTMDDYKDDDDKGSLNCADPTSERWFHGHLSGKEAEKLLTEKGKHGSFLVRESETVKGAFALSVRTGDDKGESNDGKSKVTHVLIRCQELKYDVGGGERFDSLTDLVEHYKKNPMVETLGTVLQLKQPLNTTRINAADSR

SEQ ID NO: 160 - protein, 166 aa, synthetic construct
MKIEEHHHHHHSSGKLGLNDIFEAQKIEWHESSGEDLYFQSGTTMDDYKDDDDKGGSGSMTSRRWFHPNITGVEAENLLLTRGVDGSFLARPGQSQPDFVLSVRRNGAVTHIIIQNTGDYYDLYGGEKFATLAELVQYYMEHHGQLKEKNGDVIELKYPLNCADSR

SEQ ID NO: 161 - protein, 167 aa, synthetic construct
MKIEEHHHHHHSSGKLGLNDIFEAQKIEWHESSGEDLYFQSGTTMDDYKDDDDKGGSGSMTSRRWFHPNITGVEAENLLLTRGVDGSFLARPSETVKGAFALSVRRNGAVTHILIQNTGDYYDLYGGEKFATLAELVQYYMEHHGQLKEKNGDVIELKYPLNCADSR

SEQ ID NO: 162 - protein, 180 aa, synthetic construct
MKIEEHHHHHHSSGKLGLNDIFEAQKIEWHESSGEDLYFQSGTTMDDYKDDDDKGSLNCSDPTSERWYHGHMSGGQAETLLQAKGEPWTFLVREGQSQPDFVLSVLSDQPKAGPGSPLRVTHIIVMCEGGRYTVGGLETFDSLTDLVEHFKKTGIEEASGAFVYLRQPYYATRVNAADSR

SEQ ID NO: 163 - protein, 181 aa, synthetic construct
MKIEEHHHHHHSSGKLGLNDIFEAQKIEWHESSGEDLYFQSGTTMDDYKDDDDKGSLNCSDPTSERWYHGHMSGGQAETLLQAKGEPWTFLVRESETVKGAFALSVLSDQPKAGPGSPLRVTHILVMCEGGRYTVGGLETFDSLTDLVEHFKKTGIEEASGAFVYLRQPYYATRVNAADSR

SEQ ID NO: 164 - protein, 163 aa, synthetic construct
MKIEEHHHHHHSSGKLGLNDIFEAQKIEWHESSGEDLYFQSGTTMDDYKDDDDKGGSDLPYYHGRLTKQDCETLLLKEGVDGNFLLRDSETVKGALALCVSFKNIVYTYLIFREKHGYYRIQTAEGSPKQVFPSLKELISKFEKPNQGMVVHLLKPIKRTSSR

SEQ ID NO: 165 - protein, 163 aa, synthetic construct
MKIEEHHHHHHSSGKLGLNDIFEAQKIEWHESSGEDLYFQSGTTMDDYKDDDDKGSSMAEQLRGEPWFHGKLSRREAEALLQLNGDFLVREGQSQPDYVLTGLQSGQPKHLILVDPEGVVRTKDHRFESVSHLISYHMDNHLPIISAGSELCLQQPVERKLSR

SEQ ID NO: 166 - protein, 164 aa, synthetic construct
MKIEEHHHHHHSSGKLGLNDIFEAQKIEWHESSGEDLYFQSGTTMDDYKDDDDKGSSMAEQLRGEPWFHGKLSRREAEALLQLNGDFLVRESETVKGAYALTGLQSGQPKHLLLVDPEGVVRTKDHRFESVSHLISYHMDNHLPIISAGSELCLQQPVERKLSR

SEQ ID NO: 167 - protein, 164 aa, synthetic construct
MKIEEHHHHHHSSGKLGLNDIFEAQKIEWHESSGEDLYFQSGTTMDDYKDDDDKGGSDGIQAEEWYFGKITRRESERLLLNAENPRGTFLVREGQSQPDYVLSVSDFDNAKGLNVKHYIIRKLDSGGFYITSRTQFNSLQQLVAYYSKHADGLCHRLTTVCPSR

SEQ ID NO: 168 - protein, 171 aa, synthetic construct
MKIEEHHHHHHSSGKLGLNDIFEAQKIEWHESSGEDLYFQSGTTMDDYKDDDDKGSGPPQDLSVHLWYAGPMERAGAESILANRSDGTFLVRQGQSQPDFVISIKYNVEVKHIIIMTAEGLYRITEKKAFRGLTELVEFYQQNSLKDCFKSLDTTLQFPFKEPEKRTISSR

SEQ ID NO: 169 - protein, 172 aa, synthetic construct
MKIEEHHHHHHSSGKLGLNDIFEAQKIEWHESSGEDLYFQSGTTMDDYKDDDDKGSGPPQDLSVHLWYAGPMERAGAESILANRSDGTFLVRQSETVKGAFAISIKYNVEVKHILIMTAEGLYRITEKKAFRGLTELVEFYQQNSLKDCFKSLDTTLQFPFKEPEKRTISSR

SEQ ID NO: 170 - protein, 171 aa, synthetic construct
MKIEEHHHHHHSSGKLGLNDIFEAQKIEWHESSGEDLYFQSGTTMDDYKDDDDKGSSREIDYTAYPWFAGNMERQQTDNLLKSHASGTYLIREGQSQPDFVISIKFNDEVKHIIVVEKDNWIHITEAKKFDSLLELVEYYQCHSLKESFKQLDTTLKYPYKSRERSASRSR

SEQ ID NO: 171 - protein, 172 aa, synthetic construct
MKIEEHHHHHHSSGKLGLNDIFEAQKIEWHESSGEDLYFQSGTTMDDYKDDDDKGSSREIDYTAYPWFAGNMERQQTDNLLKSHASGTYLIRESETVKGAFAISIKFNDEVKHILVVEKDNWIHITEAKKFDSLLELVEYYQCHSLKESFKQLDTTLKYPYKSRERSASRSR

SEQ ID NO: 172 - protein, 171 aa, synthetic construct
MKIEEHHHHHHSSGKLGLNDIFEAQKIEWHESSGEDLYFQSGTTMDDYKDDDDKGSPKPVDYSCQPWYAGAMERLQAETELINRVNSTYLVRHGQSQPDYVISIKYNNEAKHIIILTRDGFFHIAENRKFKSLMELVEYYKHHSLKEGFRTLDTTLQFPYKEPEHSAGQSR

SEQ ID NO: 173 - protein, 172 aa, synthetic construct
MKIEEHHHHHHSSGKLGLNDIFEAQKIEWHESSGEDLYFQSGTTMDDYKDDDDKGSPKPVDYSCQPWYAGAMERLQAETELINRVNSTYLVRHSETVKGAYAISIKYNNEAKHILILTRDGFFHIAENRKFKSLMELVEYYKHHSLKEGFRTLDTTLQFPYKEPEHSAGQSR

SEQ ID NO: 174 - protein, 181 aa, synthetic construct
MKIEEHHHHHHSSGKLGLNDIFEAQKIEWHESSGEDLYFQSGTTMDDYKDDDDKGSLNCADPTSERWFHGHLSRKEAEKLLTEKGKHGSFLVREGQSQPDFVLSVRTGDDKGESNDGKSKVTHVIIRCQELKYDVGGGERFDSLTDLVEHYKKNPMVETLGTVLQLKQPLNTTRINAADSR

SEQ ID NO: 175 - protein, 182 aa, synthetic construct
MKIEEHHHHHHSSGKLGLNDIFEAQKIEWHESSGEDLYFQSGTTMDDYKDDDDKGSLNCADPTSERWFHGHLSRKEAEKLLTEKGKHGSFLVRESETVKGAFALSVRTGDDKGESNDGKSKVTHVLIRCQELKYDVGGGERFDSLTDLVEHYKKNPMVETLGTVLQLKQPLNTTRINAADSR

SEQ ID NO: 176 - protein, 166 aa, synthetic construct
MKIEEHHHHHHSSGKLGLNDIFEAQKIEWHESSGEDLYFQSGTTMDDYKDDDDKGGSGSMTSRRWFHPNITRVEAENLLLTRGVDGSFLARPGQSQPDFVLSVRRNGAVTHIIIQNTGDYYDLYGGEKFATLAELVQYYMEHHGQLKEKNGDVIELKYPLNCADSR

SEQ ID NO: 177 - protein, 167 aa, synthetic construct
MKIEEHHHHHHSSGKLGLNDIFEAQKIEWHESSGEDLYFQSGTTMDDYKDDDDKGGSGSMTSRRWFHPNITRVEAENLLLTRGVDGSFLARPSETVKGAFALSVRRNGAVTHILIQNTGDYYDLYGGEKFATLAELVQYYMEHHGQLKEKNGDVIELKYPLNCADSR

SEQ ID NO: 178 - protein, 180 aa, synthetic construct
MKIEEHHHHHHSSGKLGLNDIFEAQKIEWHESSGEDLYFQSGTTMDDYKDDDDKGSLNCSDPTSERWYHGHMSRGQAETLLQAKGEPWTFLVREGQSQPDFVLSVLSDQPKAGPGSPLRVTHIIVMCEGGRYTVGGLETFDSLTDLVEHFKKTGIEEASGAFVYLRQPYYATRVNAADSR

SEQ ID NO: 179 - protein, 181 aa, synthetic construct
MKIEEHHHHHHSSGKLGLNDIFEAQKIEWHESSGEDLYFQSGTTMDDYKDDDDKGSLNCSDPTSERWYHGHMSRGQAETLLQAKGEPWTFLVRESETVKGAFALSVLSDQPKAGPGSPLRVTHILVMCEGGRYTVGGLETFDSLTDLVEHFKKTGIEEASGAFVYLRQPYYATRVNAADSR

SEQ ID NO: 180 - protein, 163 aa, synthetic construct
MKIEEHHHHHHSSGKLGLNDIFEAQKIEWHESSGEDLYFQSGTTMDDYKDDDDKGGSDLPYYHGRLTRQDCETLLLKEGVDGNFLLRDSETVKGALALCVSFKNIVYTYLIFREKHGYYRIQTAEGSPKQVFPSLKELISKFEKPNQGMVVHLLKPIKRTSSR

SEQ ID NO: 181 - protein, 11 aa, synthetic construct
  SITE 5: note = Phosphorylated residue
EEPQYEEIPIY

SEQ ID NO: 182 - protein, 11 aa, synthetic construct
  SITE 5: note = Phosphorylated residue
KQVEYLDLDLD

SEQ ID NO: 183 - protein, 11 aa, synthetic construct
  SITE 5: note = Phosphorylated residue
EEPQYEEIPIY

SEQ ID NO: 184 - protein, 11 aa, synthetic construct
  SITE 5: note = Phosphorylated residue
SSLSYTNPAVA

SEQ ID NO: 185 - protein, 11 aa, synthetic construct
  SITE 5: note = Phosphorylated residue
EEHVYSFPNKQ

SEQ ID NO: 186 - protein, 11 aa, synthetic construct
  SITE 5: note = Phosphorylated residue
PPPVYEPVSYH

SEQ ID NO: 187 - protein, 11 aa, synthetic construct
  SITE 5: note = Phosphorylated residue
PPPVYEPVSYH

SEQ ID NO: 188 - protein, 11 aa, synthetic construct
  SITE 5: note = Phosphorylated residue
PPPVYEPVSYH

SEQ ID NO: 189 - protein, 11 aa, synthetic construct
  SITE 5: note = Phosphorylated residue
EEPQYEEIPIY

SEQ ID NO: 190 - protein, 11 aa, synthetic construct
  SITE 5: note = Phosphorylated residue
DDPSYVNVQNL

SEQ ID NO: 191 - protein, 11 aa, synthetic construct
  SITE 5: note = Phosphorylated residue
DDPSYVNVQNL

SEQ ID NO: 192 - protein, 11 aa, synthetic construct
  SITE 5: note = Phosphorylated residue
STNEYMDMKPG

SEQ ID NO: 193 - protein, 11 aa, synthetic construct
  SITE 5: note = Phosphorylated residue
PPPVYEPVSYH

SEQ ID NO: 194 - protein, 11 aa, synthetic construct
  SITE 5: note = Phosphorylated residue
EEPQYEEIPIY

SEQ ID NO: 195 - protein, 11 aa, synthetic construct
  SITE 5: note = Phosphorylated residue
DDPSYVNVQNL

SEQ ID NO: 196 - protein, 10 aa, synthetic construct
  SITE 5: note = Phosphorylated residue
PPPTYTEVDP

SEQ ID NO: 197 - protein, 11 aa, synthetic construct
  SITE 5: note = Phosphorylated residue
KQVEYLDLDLD

SEQ ID NO: 198 - protein, 10 aa, synthetic construct
  SITE 5: note = Phosphorylated residue
PPPTYTEVDP

SEQ ID NO: 199 - protein, 11 aa, synthetic construct
  SITE 5: note = Phosphorylated residue
QGVIYSDLNLP

SEQ ID NO: 200 - protein, 11 aa, synthetic construct
  SITE 5: note = Phosphorylated residue
SLTIYAQVQKA

SEQ ID NO: 201 - protein, 11 aa, synthetic construct
  SITE 5: note = Phosphorylated residue
KQVEYLDLDLD

SEQ ID NO: 202 - protein, 10 aa, synthetic construct
  SITE 5: note = Phosphorylated residue
PPPTYTEVDP

SEQ ID NO: 203 - protein, 11 aa, synthetic construct
  SITE 5: note = Phosphorylated residue
QGVIYSDLNLP

SEQ ID NO: 204 - protein, 11 aa, synthetic construct
  SITE 6: note = Phosphorylated residue
IGEGTYGVVYK

SEQ ID NO: 205 - protein, 8 aa, synthetic construct
  SITE 4: note = Phosphorylated residue
IYQYIQSR

SEQ ID NO: 206 - protein, 11 aa, synthetic construct
  SITE 7: note = Phosphorylated residue
GEPNVSYICSR

SEQ ID NO: 207 - protein, 10 aa, synthetic construct
  SITE 5: note = Phosphorylated residue
HPDIYAVPIK

SEQ ID NO: 208 - protein, 13 aa, synthetic construct
  SITE 8: note = Phosphorylated residue
REEPEALYAAVNK

SEQ ID NO: 209 - protein, 20 aa, synthetic construct
  SITE 14: note = Phosphorylated residue
TLEPVKPPTVPNDYMTSPAR

SEQ ID NO: 210 - protein, 10 aa, synthetic construct
  SITE 7: note = Phosphorylated residue
LIEDNEYTAR

SEQ ID NO: 211 - protein, 16 aa, synthetic construct
  SITE 11: note = Phosphorylated residue
IKPSSSANAIYSLAAR

SEQ ID NO: 212 - protein, 7 aa, synthetic construct
  SITE 2: note = Phosphorylated residue
RYLENVK

SEQ ID NO: 213 - protein, 15 aa, synthetic construct
  SITE 1: note = Phosphorylated residue
YSPSQNSPIHHIPSR

SEQ ID NO: 214 - protein, 9 aa, synthetic construct
  SITE 1: note = Phosphorylated residue
YRVCNVTRR

SEQ ID NO: 215 - protein, 12 aa, synthetic construct
  SITE 12: note = Phosphorylated residue
GSPHYFSPFRPY

SEQ ID NO: 216 - protein, 10 aa, synthetic construct
  SITE 6: note = Phosphorylated residue
SESVVYADIR

SEQ ID NO: 217 - protein, 26 aa, synthetic construct
  SITE 13: note = Phosphorylated residue
SLPSGSHQGPVIYAQLDHSGGHHSDK

SEQ ID NO: 218 - protein, 11 aa, synthetic construct
  SITE 5: note = Phosphorylated residue
AVDGYVKPQIK

SEQ ID NO: 219 - protein, 10 aa, synthetic construct
  SITE 4: note = Phosphorylated residue
LGHYATQLQK

SEQ ID NO: 220 - protein, 14 aa, synthetic construct
  SITE 7: note = Phosphorylated residue
LMTGDTYTAHAGAK

SEQ ID NO: 221 - protein, 19 aa, synthetic construct
  SITE 15: note = Phosphorylated residue
IADPEHDHTGFLTEYVATR

SEQ ID NO: 222 - protein, 19 aa, synthetic construct
  SITE 15: note = Phosphorylated residue
VADPDHDHTGFLTEYVATR

SEQ ID NO: 223 - protein, 9 aa, synthetic construct
  SITE 5: note = Phosphorylated residue
GIVVYTGDR

SEQ ID NO: 224 - protein, 17 aa, synthetic construct
  SITE 2: note = Phosphorylated residue
HYSVVLPTVSHSGFLYK

SEQ ID NO: 225 - protein, 14 aa, synthetic construct
  SITE 7: note = Phosphorylated residue
TLSEVDYAPAGPAR

SEQ ID NO: 226 - protein, 9 aa, synthetic construct
  SITE 2: note = Phosphorylated residue
QYDEKLAQK

SEQ ID NO: 227 - protein, 13 aa, synthetic construct
  SITE 8: note = Phosphorylated residue
NSLETLLYKPVDR

SEQ ID NO: 228 - protein, 11 aa, synthetic construct
  SITE 6: note = Phosphorylated residue
ANGTTYATYER

SEQ ID NO: 229 - protein, 11 aa, synthetic construct
  SITE 9: note = Phosphorylated residue
ANGTTYATYER

SEQ ID NO: 230 - protein, 9 aa, synthetic construct
  SITE 7: note = Phosphorylated residue
MSLTAIYDK

SEQ ID NO: 231 - protein, 9 aa, synthetic construct
  SITE 7: note = Phosphorylated residue
FLNAESYYK

SEQ ID NO: 232 - protein, 16 aa, synthetic construct
  SITE 1: note = Phosphorylated residue
YRSDIHTEAVQAALAK

SEQ ID NO: 233 - protein, 16 aa, synthetic construct
  SITE 13: note = Phosphorylated residue
MAVILGIFGTVQYRSR

SEQ ID NO: 234 - protein, 22 aa, synthetic construct
  SITE 18: note = Phosphorylated residue
LCDFGSASHVADNDITPYLVSR

SEQ ID NO: 235 - protein, 7 aa, synthetic construct
  SITE 3: note = Phosphorylated residue
ANYQTLK

SEQ ID NO: 236 - protein, 13 aa, synthetic construct
  SITE 9: note = Phosphorylated residue
HTDDEMTGYVATR

SEQ ID NO: 237 - protein, 17 aa, synthetic construct
  SITE 11: note = Phosphorylated residue
YSRLSSTDDGYIDLQFK

SEQ ID NO: 238 - protein, 9 aa, synthetic construct
  SITE 1: note = Phosphorylated residue
YKLTVGKYR

SEQ ID NO: 239 - protein, 23 aa, synthetic construct
  SITE 6: note = Phosphorylated residue
GNFQDYVRQAVSIARQVPGTPVK

SEQ ID NO: 240 - protein, 23 aa, synthetic construct
  SITE 14: note = Phosphorylated residue
GHGQPGADAEKPFYVNVEFHHER

SEQ ID NO: 241 - protein, 10 aa, synthetic construct
  SITE 4: note = Phosphorylated residue
IEKYNVPLNR

SEQ ID NO: 242 - protein, 7 aa, synthetic construct
  SITE 2: note = Phosphorylated residue
AYSDLSR

SEQ ID NO: 243 - protein, 13 aa, synthetic construct
  SITE 4: note = Phosphorylated residue
VQIYHNPTANSFR

SEQ ID NO: 244 - protein, 9 aa, synthetic construct
  SITE 5: note = Phosphorylated residue
LRSLYANYK

SEQ ID NO: 245 - protein, 10 aa, synthetic construct
  SITE 7: note = Phosphorylated residue
VIEDNEYTAR

SEQ ID NO: 246 - protein, 8 aa, synthetic construct
  SITE 6: note = Phosphorylated residue
GLPSDYGR

SEQ ID NO: 247 - protein, 9 aa, synthetic construct
  SITE 1: note = Phosphorylated residue
YVDMSVKTK

SEQ ID NO: 248 - protein, 11 aa, synthetic construct
  SITE 9: note = Phosphorylated residue
PGQATIFVYLK

SEQ ID NO: 249 - protein, 9 aa, synthetic construct
  SITE 7: note = Phosphorylated residue
VDVNSVYQK

SEQ ID NO: 250 - protein, 14 aa, synthetic construct
  SITE 4: note = Phosphorylated residue
NEVYDNYQNWTSLK

SEQ ID NO: 251 - protein, 12 aa, synthetic construct
  SITE 4: note = Phosphorylated residue
SHGYMSTPPPVK

SEQ ID NO: 252 - protein, 11 aa, synthetic construct
  SITE 2: note = Phosphorylated residue
VYISAVLRDQR

SEQ ID NO: 253 - protein, 14 aa, synthetic construct
  SITE 9: note = Phosphorylated residue
NHTYPHLGYHHPMR

SEQ ID NO: 254 - protein, 7 aa, synthetic construct
  SITE 3: note = Phosphorylated residue
PVYPPVS

SEQ ID NO: 255 - protein, 6 aa, synthetic construct
HHHHHH

SEQ ID NO: 256 - protein, 5 aa, synthetic construct
  SITE 1: note = Phosphorylated residue
YEEIE

SEQ ID NO: 257 - protein, 8 aa, synthetic construct
  SITE 4: note = Phosphorylated residue
PPVYEPVS

SEQ ID NO: 258 - protein, 7 aa, synthetic construct
  SITE 4: note = Phosphorylated residue
PPVYEPV

(19) United States
(12) Patent Application Publication
(10) Pub. No.: US 2026/0055143 A1
(43) Pub. Date: Feb. 26, 2026

(54) HIGH AFFINITY VARIANTS OF SH2 DOMAINS

(71) Applicant: Axoiya Inc., Portola Valley, CA (US)

(72) Inventors: Sachdev S. SIDHU, Toronto (CA); Donald Gregory MARTYN, Toronto (CA); Gianluca VEGGIANI, Toronto (CA); Bradley Patrick YATES, Johnston, IA (US)

(21) Appl. No.: 19/383,102

(22) Filed: Nov. 7, 2025

Related U.S. Application Data
(63) Continuation of application No. PCT/US24/28614, filed on May 9, 2024.
(60) Provisional application No. 63/500,974, filed on May 9, 2023.

(51) Int. Cl. C07K 14/00; A61K 38/00

(52) U.S. Cl. CPC C07K 14/001; A61K 38/00; C07K 2319/60; C07K 2319/61; C07K 2319/72

(57) ABSTRACT

The present disclosure provides for high affinity variants of SH2 domains, methods of manufacturing such compositions, their use in screening, and in methods of their administration. The compositions and methods provided herein can be used for targeting protein phosphorylation of tyrosine residues as a means for identifying markers of pathology for therapeutic interventions.

### CROSS-REFERENCE

This application is a continuation of International Application No. PCT/US2024/28614, filed May 9, 2024, which claims the benefit of U.S. Provisional Application No. 63/500,974 filed May 9, 2023, each of which are incorporated by reference herein in their entirety.

### SEQUENCE LISTING

The instant application contains a Sequence Listing which has been submitted electronically in XML format and is hereby incorporated by reference in its entirety. Said XML copy, created on May 23, 2024, is named 219439-701601_SL.xml and is 271,804 bytes in size.

### BACKGROUND

Protein phosphorylation plays a significant role in regulating signal transduction pathways that are important for cell growth, migration, and replication. Aberrant protein phosphorylation has been implicated in numerous diseases and has been described as one of the major contributors to the development of cancer.

### BRIEF SUMMARY

Described herein are compositions comprising a modified Src homology 2 (SH2) domain, wherein the modified SH2 domain comprises one or more amino acid substitutions in a BC loop region, and wherein the amino acid substitutions provide for enhanced phosphorylated tyrosine (pTyr) binding to the modified SH2 domain compared to an unmodified SH2 domain. Described herein are compositions, wherein the modified SH2 domain further comprises one or more amino acid substitutions in a C anti-parallel β-sheet (βC) and a D anti-parallel β-sheet (βD), wherein the amino acid substitutions provide hydrophobic interactions with pTyr. Described herein are compositions, wherein the one or more amino acid substitutions in the C anti-parallel β-sheet (βC) comprises an Ala or a Val. Described herein are compositions, wherein the one or more amino acid substitutions in the D anti-parallel β-sheet (βD) comprises a Leu or Ile. Described herein are compositions, wherein the one or more amino acid substitutions in the C anti-parallel β-sheet (βC) comprises an Ala, and the one or more amino acid substitutions in the D anti-parallel β-sheet (βD) comprises a Leu. Described herein are compositions, wherein the one or more amino acid substitutions in the C anti-parallel β-sheet (βC) comprises a Val, and the one or more amino acid substitutions in the D anti-parallel β-sheet (βD) comprises an Ile. Described herein are compositions, wherein the modified SH2 domain further comprises an Arg residue in an αA-helix, wherein the Arg residue forms hydrogen bond with pTyr. Described herein are compositions, wherein after the one or more amino acid substitutions in the BC loop region, wherein the BC loop region comprises an amino acid sequence selected from SEQ ID NOS: 123-129. Described herein are compositions, wherein the SH2 domain having an amino acid sequence having at least 80%, at least 85%, at least 90%, or at least 95% sequence identity to the sequence selected from SEQ ID NOS: 130-180. Described herein are compositions, wherein the SH2 domain having an amino acid sequence selected from SEQ ID NOS: 130-180. Described herein are compositions, wherein the modified SH2 domain is a superbinder Fes SH2 domain variant having an amino acid sequence of SEQ ID NO: 130 (sFes¹), and the BC loop motif comprises an amino acid sequence of SEQ ID NO: 123. Described herein are compositions, wherein the modified SH2 domain is a superbinder Fes SH2 domain variant having an amino acid sequence of SEQ ID NO: 131 (sFes²), and the BC loop motif comprises an amino acid sequence of SEQ ID NO: 124. Described herein are compositions, wherein the modified SH2 domain is a superbinder Fes SH2 domain variant having an amino acid sequence of SEQ ID NO: 132 (sFes³), and the BC loop region comprises an amino acid sequence of SEQ ID NO: 125. Described herein are compositions, wherein the modified SH2 domain is a superbinder Fes SH2 domain variant having an amino acid sequence of SEQ ID NO: 133 (sFes⁴), and the BC loop region comprises an amino acid sequence of SEQ ID NO: 126. Described herein are compositions, wherein the modified SH2 domain is a superbinder Fes SH2 domain variant having an amino acid sequence of SEQ ID NO: 134 (sFes⁵), and the BC loop region comprises an amino acid sequence of SEQ ID NO: 127. Described herein are compositions, wherein the modified SH2 domain is a superbinder Fes SH2 domain variant having an amino acid sequence of SEQ ID NO: 135 (sFes⁶), and the BC loop region comprises an amino acid sequence of SEQ ID NO: 128. Described herein are compositions, wherein the compositions further comprise a detectable label. Described herein are compositions, wherein the detectable label is a radioactive label. Described herein are compositions, wherein the detectable label is a fluorescent label. Described herein are compositions, wherein the fluorescent label is fluorescein, rhodamine, or Texas Red. Described herein are compositions, wherein the fluorescent label comprises one or more fluorescent proteins. Described herein are compositions, wherein the detectable label is a biotin-based label. Described herein are compositions, wherein the detectable label is an electron-dense reagent. Described herein are compositions, wherein the detectable label is an enzyme. Described herein are compositions, wherein the enzyme is alkaline phosphatase, horseradish peroxidase, or luciferase.

Described herein is a library comprising the compositions as described herein.

Described herein are nucleic acids, wherein the nucleic acid encodes for the compositions as described herein.

Described herein are BC loops of SH2 domains having amino acid sequences selected from SEQ ID NOS: 123-129.

Described herein are methods for isolating a pTyr-containing polypeptide from a complex mixture of peptides, the methods comprising: (a) contacting a proteinaceous preparation with any one of the compositions described herein, wherein the modified SH2 domain is immobilized; and (b) isolating pTyr-containing polypeptide bound to any of the compositions of step (a). Described herein are methods, wherein the modified SH2 domain is immobilized to streptavidin beads. Described herein are methods, wherein the modified SH2 domain is biotinylated. Described herein are methods, wherein the proteinaceous preparation is from organisms. Described herein are methods, wherein the organisms are single cells, multicellular organisms, tissues, organs, or bacteria. Described herein are methods, wherein the proteinaceous preparation is from bodily fluids. Described herein are methods, wherein the bodily fluids are blood, tears, spinal fluid, synovial fluid, bronchoalveolar fluid, bronchoalveolar lavage, tissue extracts, urine, sweat, saliva, excrement, or phlegm. Described herein are methods, wherein the methods further comprise (c) characterizing the polypeptides isolated in step (b) by mass spectrometry (MS), tandem mass spectrometry (MS-MS), and/or MS3 analysis. Described herein are methods, wherein the method further comprises (d) utilizing a search program to substantially match the spectra obtained for the polypeptides isolated in step (b) during the characterization of step (c) with the spectra for a known peptide sequence, thereby identifying parent proteins of the isolated polypeptide.

Described herein are inhibitors of cellular PTK signaling pathway, wherein the inhibitors comprise the compositions described herein.

Described herein are methods of screening cancer cells, comprising: (a) lysing a sample to obtain cell lysates, wherein the sample comprises cells; (b) contacting the cell lysates and a control with any of the compositions described herein; c) collecting proteins with phosphorylated tyrosine bound to the modified Src homology 2 (SH2) domain; d) comparing a level of the proteins with phosphorylated tyrosine in the sample to a level of the proteins with phosphorylated tyrosine in the control. Described herein are methods, wherein the modified SH2 domain is labeled. Described herein are methods, wherein the modified SH2 domain is labeled with a radioactive label. Described herein are methods, wherein the detectable label is a fluorescent label. Described herein are methods, wherein the fluorescent label is fluorescein, rhodamine, or Texas Red. Described herein are methods, wherein the fluorescent label comprises one or more fluorescent proteins. Described herein are methods, wherein the modified SH2 domain is labeled with a biotin-based label. Described herein are methods, wherein the modified SH2 domain is labeled with an electron-dense reagent. Described herein are methods, wherein the modified SH2 domain is labeled with an enzyme. Described herein are methods, wherein the enzyme is alkaline phosphatase, horseradish peroxidase, or luciferase. Described herein are methods, wherein the comparing is performed by at least one of spectroscopic, photochemical, biochemical, immunochemical, or chemical techniques.

Described herein are methods of detecting a protein tyrosine kinase in a biological sample comprising: (a) contacting the biological sample with any of the compositions described herein; and (b) applying an assay for detecting the detectable label in the biological sample after the contacting, wherein detection of the label in the biological sample indicates the presence of the protein kinase in the biological sample. Described herein are methods, wherein the assay applies quantifiable or semi-quantifiable spectroscopic, photochemical, biochemical, immunochemical, or chemical means technique. Described herein are methods, wherein the assay is mass spectrometry (MS), tandem mass spectrometry (MS-MS), MS3 analysis, SPECT, CT and PET imaging, enzyme linked immunosorbent assay (ELISA), or luciferase assay.

Described herein are methods of diagnosing a disorder or condition associated with altered levels of pTyr in a subject, said method comprising: (a) contacting a sample taken from said subject with one or more of the compositions of described herein wherein the modified SH2 domains are immobilized; (b) isolating polypeptides bound by the at least one immobilized peptide, thereby isolating pTyr-containing polypeptides from the sample; (c) measuring the level of pTyr-containing polypeptides in the sample using a spectroscopic, photochemical, biochemical, immunochemical, or chemical technique; (d) comparing the level measured in (c) with a normal control level; and (e) determining whether the subject has the disorder or condition based on the comparison of (d).

Described herein are pharmaceutical compositions comprising the compositions described herein. Described herein are pharmaceutical compositions, wherein the pharmaceutical composition described herein further comprising a solubilizing agent and an excipient. Described herein are pharmaceutical compositions, wherein the excipient comprises one or more of a buffering agent, a stabilizer, an antioxidant, a binder, a diluent, a dispersing agent, a rate controlling agent, a lubricant, a glidant, a disintegrant, a plasticizer, a preservative, or any combinations thereof. Described herein are pharmaceutical compositions, wherein the excipient comprises di-sodium hydrogen phosphate dihydrate, sodium dihydrogen phosphate dihydrate, sodium chloride, myo-inositol, sorbitol, or any combinations thereof. Described herein are pharmaceutical compositions, wherein the pharmaceutical composition further comprises one or more of a preservative, a diluent, and a carrier. Described herein are pharmaceutical compositions, wherein the pharmaceutical composition further comprises an additional active ingredient or salt thereof.

Described herein are methods of treating a disorder associated with protein phosphorylation, comprising administering to a subject any of the pharmaceutical compositions described herein. Described herein are methods, wherein the disease is an allergic disease. Described herein are methods, wherein the disease is an autoimmune disease. Described herein are methods, wherein the disorder is cancer.

Described herein are methods of producing a modified SH2 domain having equal or increased binding affinity to a pTyr-containing ligand relative to its unmodified version of the SH2 domain (SH2 domain to be modified), the SH2 domain to be modified domain having a pTyr binding pocket (αA2), a three-strand anti-parallel β-sheet including a B anti-parallel β-sheet (βB), a C anti-parallel β-sheet (βC) and a D anti-parallel β-sheet (βD), the three-strand anti-parallel β-sheet being flanked by a two α-helices, the BB being separated from RC by a BC loop, the methods comprising: a) aligning the amino acid sequence of the SH2 domain to be modified using a suitable alignment algorithm to multiple SH2 domain sequences to determine the amino acid positions of αA2, the three-strand anti-parallel β-sheet and the two α-helices; and at least one or both of b1) replacing the BC loop (amino acid positions 59 to 68) of the SH2 domain to be modified with a BC loop motif of a superbinder SH2 domain that exhibits increased binding affinity for the pTyr-containing ligand relative to a wild-type version of the SH2 domain superbinder, and b2) when the SH2 domain to be modified lacks an Arg residue at the αA2 position (position 33), substituting the amino acid at the position αA2 of the SH2 domain to be modified with an Arg residue, thereby producing the modified SH2 domain having equal or increased binding affinity to the pTyr-containing ligand. In some aspects, when the SH2 domain to be modified contains hydrophilic amino acid residues at amino acid position 70 (PC2) and at amino acid position 102 (βD6), then the method further comprises replacing each of said hydrophilic amino acid residues at position βC2 and at position βD6 with a hydrophobic amino acid residue, wherein the amino acid position 70 and the amino acid position 102 of the SH2 domain to be modified are determined from the sequence alignment of (a). Described herein are methods, wherein the superbinder SH2 domain is a superbinder Src SH2 domain (sSrc, SEQ ID NO: 136), and the BC loop motif has an amino acid sequence of SETVKGA (SEQ ID NO: 129). Described herein are methods, wherein the methods further comprise replacing the amino acid at position βC2 (position 70) and the amino acid at position βD6 (position 102) of the SH2 domain to be modified with an Ala residue and a Leu residue respectively, wherein the amino acid position 70 and the amino acid position 102 of the SH2 domain to be modified are determined from the sequence alignment of (a). Described herein are methods, wherein the superbinder SH2 domain is a superbinder Fes SH2 domain variant (sFes1) having an amino acid sequence of SEQ ID NO: 130, and the BC loop motif has an amino acid sequence of GQSQPD (SEQ ID NO: 123). Described herein are methods, wherein the superbinder SH2 domain is a superbinder Fes SH2 domain variant (sFes2) having an amino acid sequence of SEQ ID NO: 131, and the BC loop motif has an amino acid sequence of RQRKQE (SEQ ID NO: 124). Described herein are methods, wherein the superbinder SH2 domain is a superbinder Fes SH2 domain variant having an amino acid sequence of SEQ ID NO: 132 (sFes3), and the BC loop motif has an amino acid sequence of SPRIQE (SEQ ID NO: 125). Described herein are methods, wherein the superbinder SH2 domain is a superbinder Fes SH2 domain variant having an amino acid sequence of SEQ ID NO: 133 (sFes4), and the BC loop motif has an amino acid sequence of SQGRKV (SEQ ID NO: 126). Described herein are methods, wherein the superbinder SH2 domain is a superbinder Fes SH2 domain variant having an amino acid sequence of SEQ ID NO: 134 (sFes5), and the BC loop motif has an amino acid sequence of SISKQG (SEQ ID NO:127). Described herein are methods, wherein the superbinder SH2 domain is a superbinder Fes SH2 domain variant having an amino acid sequence of SEQ ID NO: 135 (sFes6), and the BC loop motif has an amino acid sequence of SQTYPG (SEQ ID NO: 128). Described herein are methods, wherein the methods further comprise replacing the amino acid at position βC2 (position 70) and the amino acid at position βD6 (position 102) of the SH2 domain to be modified with a Val residue and an Ile residue respectively, wherein the amino acid position 70 and the amino acid position 102 of the SH2 domain to be modified are determined from the sequence alignment of (a). Described herein are methods, wherein the multiple SH2 domain sequences include the SH2 domain sequences found in Table 1 (SEQ ID NOS: 1-122).

Described herein are methods of producing a modified SH2 domain comprising: aligning an amino acid sequence of an SH2 domain with its homologs that specifically bind to phosphorylated proteins; determining amino acid residues required for the binding using a computer program; and modifying at least one of the amino acid residues required for the binding, wherein the modification provides for increased binding affinity of the SH2 domain to phosphorylated proteins as compared to the unmodified SH2 domain. Described herein are methods, wherein the computer program comprises a biomolecular modeling program or design algorithms. Described herein are methods, wherein the computer program is RoseTTAFold ALL-Atom (RFAA).

### BRIEF DESCRIPTION OF THE DRAWINGS

The novel features of the disclosure are set forth with particularity in the appended claims. A better understanding of the features and advantages of this disclosure will be obtained by reference to the following detailed description that sets forth illustrative implementations, in which the principles of this disclosure are utilized, and the accompanying drawings of which:

FIGS. 1A-1B show, in one example, diagrams of high affinity variants of the Fes SH2 domain. FIG. 1A illustrates the structure of Fes^{wt} (PDB ID: 1WQU) depicted as a dark grey cartoon skeleton and light grey surface representation. FIG. 1B shows the sequence of the Fes SH2 domain (SEQ ID NO: 21) with indicated secondary structure, individual residues chosen for mutagenesis (bold & underlined) and the regions selected for randomized mutagenesis.

FIGS. 2A-2C illustrate, in one example, Src and Fes superbinder motif swapping. FIG. 2A illustrates the superimposition of Cα co-ordinates of Srcwt (PDC ID: 1HCT) and Feswt (PDB ID: 1WQU) rendered in PyMol. FIGS. 2B-2C show the fold increase in binding affinity of SH2 domain variants over their (wild-type) (wt) counterparts. Data are an average of 2-4 biological replicates ±SEM and are shown as fold change over Srcwt and Feswt, respectively.

FIGS. 3A-3B show, in one example, plots of motif grafting into a diverse set of SH2 domains and analysis of the differences in affinity among different SH2 domains. FIG. 3A shows the relative fold change in binding affinity of variant SH2 domains over their wt counterparts as determined by IC₅₀ values. X axis ais the logarithm of fold change in affinity of each modified SH2 domain over the wildtype counterpart. Y axis lays every SH domain tested. FIG. 3B shows the relative fold change in binding affinity of SH2 domain variants with an additional mutation to Arginine within the αA1-helix over their wt counterparts as determined by IC₅₀ values. Data are an average of 2-3 biological replicates ±SEM and are shown as fold change over their respective wt form. X axis is the logarithm of fold change in affinity of each modified SH2 domain over the wildtype counterpart. Y axis lays out the modified SH2 domains Ptn11_N, Ptn11_C, Ptn6_C, and SH2D1B.

FIG. 4 shows, in one example, a schematic workflow for TMT-based quantitative proteomics analyses using Mass Spectrometry.

FIG. 5 shows, in one example, the IC50 values for Src and Fes-SH2 variants binding to phosphopeptides and the fold changes as compared to the wild type.

FIGS. 6A-6D illustrate, in one example, structural comparison of Src-SH2 and its superbinders. FIG. 6A shows the superposition of Src-SH2 and its variants. The left panel depicts the following structures: unbound vSrc (PDB ID: 1BKL), unbound sSrc1 (PDB ID: 4F59), bound Srcwt (PDB ID: 1HCT), and bound sSrc1 (PDB ID: 4F5B). The right panel depicts the following structures: bound Srcwt, bound sSrc1 and bound sSrcF. Structures were aligned based on Ca coordinates using the ALIGN function in PyMol. FIG. 6B shows superposition of the pTyr-binding pockets of unbound vSrc and unbound sSrc1. FIG. 6C shows superposition of the pTyr-binding pockets of bound Srcwt and bound sSrc1. Hydrogen bonds are shown as dashed lines and numbers refer to interactions described in the main text. FIG. 6D shows superposition of the pTyr-binding pockets of bound sSrc1 and bound sSrcF.

FIG. 7 shows, in one example, plots of grafting of sSrc1 and sFes1 superbinder motifs into diverse SH2 domains and IC₅₀ values for each variant. Data are an average of 3-4 repetitions ±SEM. IC₅₀ values for which curve fitting could not be performed are listed as >20000 nM. “NDI” indicates “no detectable inhibition”.

### DETAILED DESCRIPTION

Mapping protein phosphorylation is of importance for uncovering pathogenic signaling pathways and identifying novel therapeutic strategies. Src Homology 2 (SH2) domains naturally bind phosphotyrosine (pTyr) containing proteins (pTyr-proteins) in cells to mediate pTyr dependent signal transduction networks. The binding affinity of natural SH2 domains to pTyr-proteins may be low. Thus, making modified SH2 domains with increased binding affinity is useful for targeting protein phosphorylation of tyrosine residues to identify markers of pathology for therapeutic interventions.

Provided herein are compositions and methods for targeting protein phosphorylation by modified SH2 domains. Compositions described herein can comprise the modified SH2 domains as described herein. Nucleic acids encoding the modified SH2 domains provided herein can comprise DNA, RNA, nucleic acid analogues, or any combination thereof. Examples described herein include (1) modified SH2 domains, (2) methods of making modified SH2 domains, (3) phosphorylation capturing reagents, (4) pharmaceutical compositions, (5) conditions for treatment, (6) Diagnosis, and (7) additional applications.

### Definitions

Unless defined otherwise, all technical and scientific terms used herein have the same meaning as commonly understood by one of ordinary skill in the art to which this disclosure belongs. Also, unless indicated otherwise, except within the claims, the use of “or” includes “and” and vice versa. Non-limiting terms are not to be construed as limiting unless expressly stated or the context clearly indicates otherwise (in some aspects, “containing”, “including”, “having” and “comprising” typically indicate “including without limitation”). Examples of limiting terms include “consisting of” and “consisting essentially of”. Singular forms including in the claims such as “a”, “an” and “the” include the plural reference unless expressly stated otherwise.

Unless specifically stated or obvious from context, as used herein, the term “about” in reference to a number or range of numbers is understood to include small fluctuations, referring to the stated number and numbers +/−10% thereof.

Unless specifically stated or obvious from context, as used herein, the term “substantially match” is understood to describes a situation that the resulting analysis and comparison is performed to identify resources that are the same; however, in practice the match can correspond to a set of resources that sufficiently similar for the methods describe herein.

The following standard one letter and three letter abbreviations for the amino acid residues used throughout the specification: A, Ala—alanine; R, Arg—Arginine; N, Asn—Asparagine; D, Asp—Aspartic acid; C, Cys—Cysteine; Q, Gln—Glutamine; E, Glu—Glutamic acid; G, Gly—Glycine; H, His—Histidine; I, Ile—Isoleucine; L, Leu—Leucine; K, Lys—Lysine; M, Met—Methionine; F, Phe—Phenyalanine; P, Pro—Proline; S, Ser—Serine; T, Thr—Threonine; W, Trp—Tryptophan; Y, Tyr—Tyrosine; and V, Val—Valine.

The term “pTyr” herein refers to phosphotyrosine. The term “pTyr-containing polypeptide” refers to a molecule that comprises a pTyr-containing peptide or peptide fragment.

The term “isolated peptide” or “isolated DNA” refers to a peptide or DNA molecule that has been produced and removed from the source that produced the peptide or DNA, such as recombinant cells or synthesis reactants. The term includes, without limitation, recombinant or cloned DNA isolates and chemically synthesized analogs or analogs biologically synthesized by heterologous systems.

The term “peptide” or “polypeptide” or “oligopeptide” as used herein is defined as a chain of amino acid residues, usually having a defined sequence. As used herein the term “peptide” is mutually inclusive of the terms “polypeptides”, “peptides” and “proteins”.

The term “parent SH2 domain” refers to a polypeptide having at least about 50% (e.g., at least about 60%, about 70%, about 80%, about 90%, or higher) homology to a wt SH2 domain derived from a human protein that contains an SH2 domain. The human genome encodes 122 SH2 domains (Table 1; SEQ ID NOS: 1-122) within 112 different proteins with many more in other species. Homology can be determined by comparing a position in each sequence which is aligned for purposes of comparison. When a position in the compared polypeptide is occupied by the same base or amino acid, then the molecules are homologous at that position. A degree of homology between sequences is a function of the number of matching or homologous positions shared by the sequences.

The terms “modified SH2 domain”, “variant SH2 domain”, “SH2 Variant”, “SH2 monobody” are used indistinguishably to refer to a parent SH2 domain that has been modified for affinity enhancement according to the methods of the present disclosure. In some aspects, a modified SH2 domain of the present disclosure has equal or more affinity to pTyr than its parent SH2. In some aspects of the present disclosure, a modified SH2 domain that is intended for use in human body for purposes including clinical or diagnostic use, may be designed from a human SH2 domain as a parent SH2 domain, in order to minimize the possibility of immune response that may be caused by supplementation of the variant SH2 domain to the body.

Table 2 includes the sequences of human SH2 domains (Table 1; SEQ IDs: 1-122), which were downloaded from the Universal Protein Resource (UniProt; https://www.uniprot.org/) and are aligned using the Constraint Based Alignment Tool (COBALT)12 from NCBI (National Center for Biotechnology Information; https://www.ncbi.nlm.nih.gov/tools/cobalt/re_cobalt.cgi). The sequence alignment was visualized using Jalview software (https://www.jalview.org/). Highlighted using grey boxes in the alignment is the αA-2 (position 33), BC Loop (positions 59-68), βC-2 (position 70) and βD-6 (position 102) that correspond to residues used to make SH2 superbinders using the grafting technique of the present disclosure. Note that dashes (“-”) depict gaps in the sequence alignment.

The term “Superbinder SH2” or “sSH2,” refers to a SH2 domain having ultra-high affinity for pTyr (range of about 0.8-1 μM). SH2 domains are comprised of a central anti-parallel β-sheet having 3 β-strands: a B β-strand (βB), a C β-strand (RC) and a D β-strand (βD). The three β strands are linked to one another by a loop. In some aspects, the βC is separated from βB by a BC loop. The central anti-parallel β-sheet is flanked by two α-helices positioned on either side. The pTyr-binding pocket is characterized by a highly conserved Arg residue in the βB6 position that coordinates the phosphate moiety of the pTyr residue, and this interaction contributes most of the total free energy of the SH2-ligand interaction. Adjacent to the pTyr-binding pocket are a series of hydrophobic pockets that interact with side chains of amino acids C-terminal to the pTyr residue. These hydrophobic pockets dictate SH2 ligand specificity and are rendered either accessible, or occluded, by the cooperative action of the EF and BG-loops. Thus, the SH2 domain employs a two-pronged binding mode that is dependant on the presence of pTyr and the type of amino acids C-terminal to the pTyr residue itself. Therefore, different SH2 domains bind different sets of pTyr-containing peptides and, therefore, can enrich a broader subset of the pTyr phosphoproteome for analysis than traditional antibody-based methods.

The term “mutation,” as used herein, refers to a deletion, an insertion of a heterologous nucleic acid, an inversion, or a substitution, including an open reading frame ablating mutations as commonly understood in the art.

The term “gene,” as used herein, refers to a segment of nucleic acid that encodes for an individual protein or RNA (also referred to as a “coding sequence” or “coding region”), optionally together with associated regulatory regions such as promoters, operators, terminators, and the like, which may be located upstream or downstream of the coding sequence.

A “promoter,” as used herein, refers a control sequence that is a region of a nucleic acid sequence at which initiation and rate of transcription are controlled. In some aspects, a promoter may contain genetic elements at which regulatory proteins and molecules may bind such as RNA polymerase and other transcription factors. The terms “operatively positioned,” “operatively linked,” “under control” and “under transcriptional control” can mean that a promoter is in a correct functional location and/or orientation in relation to a nucleic acid sequence to control transcriptional initiation and/or expression of that sequence. In some aspects, a promoter may or may not be used in conjunction with an “enhancer,” which refers to a cis-acting regulatory sequence involved in the transcriptional activation of a nucleic acid sequence.

The term “homology,” as used herein, refers to calculations of “homology” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences is a function of the number of identical positions shared by the sequences (i.e., % homology=# of identical positions/total # of positions×100). In some aspects, a position in the first sequence is occupied by the same nucleotide as the corresponding position in the second sequence, then the molecules are identical at that position. The percent homology between the two sequences is a function of the number of identical positions shared by the sequences, taking into account the number of gaps, and the length of each gap, which need to be introduced for optimal alignment of the two sequences. In some aspects, the length of a sequence aligned for comparison purposes is at least about 30%, at least about 40%, about 50%, about 60%, about 65%, about 70%, about 75%, about 80%, about 85%, about 90%, about 91%, about 92%, about 93%, about 94%, about 95%, about 96%, about 97%, about 98%, or 99%, or higher, of the length of the reference sequence. A BLAST® search may determine homology between two sequences. The homology can be between the entire lengths of two sequences or between fractions of the entire lengths of two sequences. The two sequences can be genes, nucleotides sequences, protein sequences, peptide sequences, amino acid sequences, or fragments thereof. In some aspects, the actual comparison of the two sequences is accomplished by well-known methods, using a mathematical algorithm. When utilizing BLAST and Gapped BLAST programs, any relevant parameters of the respective programs (e.g., NBLAST) can be used. In some aspects, parameters for sequence comparison can be set at score=100, word length=12, or can be varied (e.g., W=5 or W=20). Other examples include the algorithm of Myers and Miller, CABIOS, ADVANCE, ADAM, BLAT, and FASTA.

The term “subject” refers to an animal, including, but not limited to, a primate (e.g., human), cow, sheep, goat, horse, dog, cat, rabbit, rat, or mouse. In some aspects, the terms “subject” and “patient” are used interchangeably herein in reference to a mammalian subject. In some aspects, the subject is human.

The terms “treat,” “treating,” and “treatment” refers to alleviating or abrogating a disorder, disease, or condition; or one or more of the symptoms associated with the disorder, disease, or condition; or alleviating or eradicating the cause(s) of the disorder, disease, or condition itself.

The term “therapeutically effective amount” refers to the amount of a compound that, when administered, can be sufficient to treat one or more of the symptoms of the disorder, disease, or condition being treated.

The term “oncolytic,” as used herein, refers to killing of cancer or tumor cells by an agent, such as an oncolytic poxvirus, such as an oncolytic vaccinia virus, e.g., through the direct lysis of said cells, by stimulating immune response towards said cells, apoptosis, expression of toxic proteins, autophagy and shutdown of protein synthesis, induction of anti-tumoral immunity, or any combinations thereof. The direct lysis of the cancer or tumor cells infected by the agent, such as an oncolytic vaccinia virus, can be a result of replication of the virus within said cells. In certain examples, the term “oncolytic,” can refer to killing of cancer or tumor cells without lysis of said cells.

The term “oncolytic virus” as used herein refers to a virus that preferentially infects and kills tumor cells. In some implementations, the oncolytic viruses can include, but are not limited to, (i) viruses that naturally replicate preferentially in cancer cells and are non-pathogenic in humans often due to elevated sensitivity to innate antiviral signaling or dependence on oncogenic signaling pathways; and (ii) viruses that are genetically-manipulated for use. In some implementations, the oncolytic virus can be a measles virus, a poliovirus, a poxvirus, a vaccinia virus, an adenovirus, an adeno associated virus, a herpes simplex virus, a vesicular stomatitis virus, a reovirus, a Newcastle disease virus, a senecavirus, a lentivirus, a mengovirus, or a myxoma virus. In certain implementations, the oncolytic virus can be a poxvirus. In certain implementations, the oncolytic virus can be a vaccinia virus.

The term “modified oncolytic virus” as used herein refers to an oncolytic virus that comprises a modification to its constituent, such as, but not limited to, a modification in the native genome (“backbone”) of the virus like a mutation or a deletion of a viral gene, introduction of an exogenous nucleic acid, a chemical modification of a viral nucleic acid or a viral protein, and introduction of an exogenous protein or modified viral protein to the viral capsid. In general, oncolytic viruses may be modified (also known as “engineered”) in order to gain improved therapeutic effects against tumor cells. In some implementations, the modified oncolytic virus can be a modified poxvirus. In some implementations, the modified oncolytic virus can be a modified poxvirus. In some implementations, the modified oncolytic virus can be a modified vaccinia virus.

The terms “systemic delivery,” and “systemic administration,” used interchangeably herein, refers to a route of administration of medication, oncolytic virus or other substances into the circulatory system. The systemic administration may comprise oral administration, intraperitoneal administration, parenteral administration, intranasal administration, sublingual administration, rectal administration, transdermal administration, intra-arterial administration, or any combinations thereof.

### Modified SH2 Domain

i. BC Loop Substitutions

Provided here are compositions comprising modified SH2 domains. In some aspects, the modified SH2 domains comprise one or more amino acid substitutions in a BC loop region. In some aspects, the one or more amino acid substitutions provide for enhanced phosphorylated tyrosine (pTyr) binding to the modified SH2 domain compared to an unmodified SH2 domain. In some aspects, the modified SH 2 domain comprises a BC loop of a SH2 domain superbinder.

In some aspects, the modified SH2 domains further comprise one or more amino acid substitutions in the backside of the pTyr binding pocket in the SH2 domain fold of the parent domain, wherein the one or more amino acid substitutions provide hydrophobic interactions with pTyr. In some aspects, the one or more amino acid substitutions comprises an Ala or a Val. In some aspects, the one or more amino acid substitutions comprises a Leu or an Ile. In some aspects, the one or more amino acid substitutions comprises an Ala and a Leu. In some aspects, the one or more amino acid substitutions comprises a Leu and an Ile.

In some aspects, a modified SH2 domain described herein comprises a superbinder SH domain (sSH2). In some aspects, the sSH2 is sFes¹ (SEQ ID: 130). In some aspects, the modified SH2 domains comprise the sFes¹ BC loop. In some aspects, the sFes¹ BC loop comprises the amino acid sequence of SEQ ID NO: 123 (GQSQPD). In some aspects, the modified SH2 domains further comprise a hydrophobic amino acid at position βC-2 (position 70). In some aspects, the modified SH2 domains further comprise a hydrophobic amino acid residue at βD-6 (position 102), In some aspects, the modified SH2 domain comprises a Val residue at position βC-2 (position 70). In some aspects, the modified SH2 domains comprise a Ile residue at position βD-6 (position 102). In some aspects, the modified SH2 domains comprise a Val residue at position βC-2 (position 70) and a Ile residue at position βD-6 (position 102).

In some aspects, the sSH2 is sFes² (SEQ ID: 131). In some aspects, the method comprises grafting the sFes² BC loop domain. In some aspects, the sFes² BC loop domain comprises the amino acid sequence of SEQ ID NO: 124 (RQRKQE). In some aspects, the modified SH2 domains further comprise a hydrophobic amino acid at position βC-2 (position 70). In some aspects, the modified SH2 domains further comprise a hydrophobic amino acid residue at βD-6 (position 102), In some aspects, the modified SH2 domain comprises a Val residue at position βC-2 (position 70). In some aspects, the modified SH2 domains comprise a Ile residue at position βD-6 (position 102). In some aspects, the modified SH2 domains comprise a Val residue at position βC-2 (position 70) and a Ile residue at position βD-6 (position 102).

In some aspects, the sSH2 is sFes³ (SEQ ID: 132). In some aspects, the modified SH2 domains comprise the sFes³ BC loop domain. In some aspects, the sFes³ BC loop domain comprises the amino acid sequence of SEQ ID NO: 125 (SPRIQE). In some aspects, the modified SH2 domains further comprise a hydrophobic amino acid at position βC-2 (position 70). In some aspects, the modified SH2 domains further comprise a hydrophobic amino acid residue at βD-6 (position 102), In some aspects, the modified SH2 domain comprises a Val residue at position βC-2 (position 70). In some aspects, the modified SH2 domains comprise a Ile residue at position βD-6 (position 102). In some aspects, the modified SH2 domains comprise a Val residue at position βC-2 (position 70) and a Ile residue at position βD-6 (position 102).

In some aspects, the sSH2 is sFes⁴ (SEQ ID: 133). In some aspects, the modified SH2 domains comprise the sFes⁴ BC loop domain. In some aspects, the modified SH2 domains comprise the amino acid sequence of SEQ ID NO: 126 (SQGRKV). In some aspects, the modified SH2 domains further comprise a hydrophobic amino acid at position βC-2 (position 70). In some aspects, the modified SH2 domains further comprise a hydrophobic amino acid residue at βD-6 (position 102), In some aspects, the modified SH2 domain comprises a Val residue at position βC-2 (position 70). In some aspects, the modified SH2 domains comprise a Ile residue at position βD-6 (position 102). In some aspects, the modified SH2 domains comprise a Val residue at position βC-2 (position 70) and a Ile residue at position βD-6 (position 102).

In some aspects, the sSH2 is sFes⁵ (SEQ ID: 134). In some aspects, the modified SH2 domain comprises the sFes⁵ BC loop. In some aspects, the modified SH2 domain comprises SEQ ID NO: 127 (SISKQG). In some aspects, the modified SH2 domain further comprises a hydrophobic amino acid at position βC-2 (position 70). In some aspects, the modified SH2 domains further comprise a hydrophobic amino acid residue at βD-6 (position 102). In some aspects, the modified SH2 domain comprises a Val residue at position βC-2 (position 70). In some aspects, the modified SH2 domains comprise a Ile residue at position βD-6 (position 102). In some aspects, the modified SH2 domains comprise a Val residue at position βC-2 (position 70) and a Ile residue at position βD-6 (position 102).

In some aspects, the sSH2 is sFes⁶ (SEQ ID: 135). In some aspects, the modified SH2 domains comprise the sFes⁶ BC loop. In some aspects, the sFes⁶ BC loop comprises SEQ ID NO: 128 (SQTYPG). In some aspects, the modified SH2 domains further comprise a hydrophobic amino acid at position βC-2 (position 70). In some aspects, the modified SH2 domains further comprise a hydrophobic amino acid residue at βD-6 (position 102), In some aspects, the modified SH2 domain comprises a Val residue at position βC-2 (position 70). In some aspects, the modified SH2 domains comprise a Ile residue at position βD-6 (position 102). In some aspects, the modified SH2 domains comprise a Val residue at position βC-2 (position 70) and a Ile residue at position βD-6 (position 102).

In some aspects, the sSH2 is sSrc (SEQ ID: 136). In some aspects, the modified SH2 domain comprises the sSrc BC loop domain. In some aspects, the sSrc BC loop comprises SEQ ID NO: 129 (SETVKGA). In some aspects, the modified SH2 domain further comprises a hydrophobic amino acid at position βC-2 (position 70). In some aspects, the modified SH2 domain further comprises a hydrophobic amino acid residue at βD-6 (position 102). In some aspects, the modified SH2 domain comprises an Ala residue at position βC-2 (position 70). In some aspects, the modified SH2 domain comprises a Leu residue at position βD-6 (position 102). In some aspects, the modified SH2 domain comprises an Ala residue at position βC-2 (position 70) and a Leu residue at position βD-6 (position 102).

ii. Arg Residue

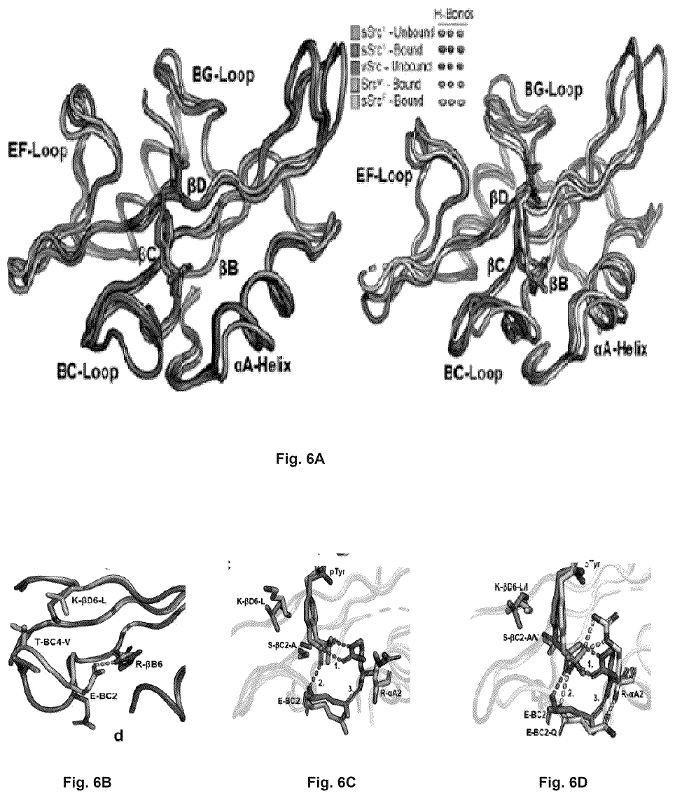

Some SH2 domains comprise an Arg residue in the αA-2 (position 33) that forms hydrogen bond with the pTyr phosphoryl group (FIGS. 6A-6D), providing a stabilization effect. Thus, in some aspects, the modified SH2 domain comprises an Arg residue at position αA-2 (position 33) of the SH2 domain. In some aspects, the Arg residue forms hydrogen bond with pTyr.

In some aspects, the SH2 domain disclosed herein comprises an amino acid sequence having at least 80%, at least 85%, at least 90%, at least 95%, at least 98%, or at least 99% sequence identity to the sequence selected from SEQ ID NOS: 130-180. In some aspects, the SH2 domain comprises an amino acid sequence selected from SEQ ID NOS: 130-180. In some aspects, the modified SH2 domain comprises an amino acid sequence selected from SEQ ID NOS: 130-180.

In some aspects, the modified SH2 domain disclosed herein is synthesized by any method in the art of peptide synthesis including solid phase synthesis and encompassing both L/D-enantiomers of the amino acids and non-canonical amino acids or synthesis in homogenous solution to generate synthetic peptides.

In some aspects, the modified SH2 domain described herein is made by the use of recombinant DNA techniques available to one skilled in the art. Nucleic acid sequences which encode for the selected peptides of the disclosure are incorporated in a known manner into appropriate expression vectors (i.e., recombinant expression vectors). Possible expression vectors include (but are not limited to) cosmids, bacmids, plasmids, or modified viruses (e.g., replication defective retroviruses, adenoviruses and adeno-associated viruses, lentiviruses; herpes viruses, poxviruses), so long as the vector is compatible with the host cell used. The expression “vector is compatible with the host cell” is defined as contemplating that the expression vector(s) contain a nucleic acid molecule of the disclosure (hereinafter described) and attendant regulatory sequence(s) selected on the basis of the host cell(s) to be used for expression, said regulatory sequence(s) being operatively linked to the nucleic acid molecule. “Operatively linked” is intended to mean that the nucleic acid is linked to regulatory sequence(s) in a manner which allows expression of the nucleic acid. Suitable regulatory sequences may be derived from a variety of sources, including bacteria), fungal, or viral genes. Selection of appropriate regulatory sequence(s) is dependent on the host cell(s) chosen and may be readily accomplished by one of ordinary skill in the art. Examples of such regulatory sequences include the following: a transcriptional promoter and enhancer, RNA polymerase binding sequence, or a ribosomal binding sequence (including a translation initiation signal). Depending on the host cell chosen and the expression vector employed, other additional sequences (such as an origin of replication, additional DNA restriction sites, enhancers, and sequences conferring inducibility of transcription) may be incorporated into the expression vector.

Also provided here are vectors which comprise nucleic acids coding for at least one sSH2 of the present disclosure. In some aspects, the polypeptides comprising the sSH2's provided herein may also be produced using cell free protein expression systems.

In some aspects, the modified SH2 domains of the present disclosure is provided with a cell membrane penetrating peptide, such as a TAT protein transduction domain. TAT-fusions have been shown to cross cell membranes and, in some instances, blood barriers.

In some aspects, the modified SH2 domains described herein is labelled with a label to facilitate their detection in a variety of assays as is understood by one of skill in the art. Such labels may include but are not limited to radioactive label, biotin, a magnetic label, a paramagnetic label, a radiodense label, an enzyme, a hapten, a cytotoxic label, a luminescent label, a fluorescent label and nucleic acid labels. In some aspects, the modified SH2 domains described herein couple to bovine serum albumin (BSA) or keyhole limpet haemocyanin. The peptides may be covalently or non-covalently coupled to a solid carrier such as a microsphere of gold or polystyrene, a slide, chip or to a wall of a microtiter plate. The peptide may be labelled directly or indirectly with a label selected from but not limited to biotin, fluorescein and an enzyme such as horseradish peroxidase alkaline phosphatase, or luciferase. In some aspects, the modified SH2 domains are preceded by a Biotin N-terminal sequence that may facilitate peptide concentration determination by OD280 (of Tyr or Y) measurement.

### Methods of Making Modified SH2 Domains

Provided herein are in silico methods of producing to post-translational modifications (PTMs). In some aspects, the method comprises: aligning an amino acid sequence of an SH2 domain with its homologs that specifically bind to phosphorylated proteins; determining amino acid residues required for the binding using a computer program comprising biomolecular modeling and design algorithms; and modifying at least one of the amino acid residues required for the binding, wherein the modification provides for increased binding affinity of the SH2 domain to phosphorylated proteins as compared to the unmodified protein domain. In some aspects, the computer program comprises a biomolecular modeling program and/or design algorithms. In some aspects, the computer program is RoseTTAFold All-Atom.

In some aspects, the method comprises making modified SH2 domains having enhanced binding affinity to a pTyr-containing polypeptide, the method comprising one or any combination of the following processes: (a) replacing the BC loop of a parent SH2 domain with a BC loop of a SH2 domain superbinder; (b) replacing the residue in a conserved alpha-helix with an Arg, wherein the Arg forms hydrogen bond with a pTyr; and (c) replacing one or more residues in the backside of a pTyr binding pocket in the SH2 domain.

i. BC Loop Grafting

Provided here are methods of producing a modified Src homology 2 (SH2) domain which exhibits equal or increased binding affinity to a pTyr-containing ligand relative to its unmodified version, the methods comprising: replacing the BC loop of the unmodified version of the SH2 domain (i.e., the SH2 domain to be modified) with the BC loop of a SH2 domain superbinder (sSH2) that exhibits increased binding affinity for the pTyr-containing ligand relative to a wild-type version of the SH2 domain superbinder.

In some aspects, methods described herein further comprise making one or more amino acid substitutions in the backside of the pTyr binding pocket in the SH2 domain fold of the parent domain, wherein the one or more amino acid substitutions provide hydrophobic interactions with pTyr. In some aspects, the one or more amino acid substitutions comprises an Ala or a Val. In some aspects, the one or more amino acid substitutions comprises a Leu or an Ile. In some aspects, the one or more amino acid substitutions comprises an Ala and a Leu. In some aspects, the one or more amino acid substitutions comprises a Leu and an Ile.

In some aspects, the sSH2 is sFes¹ (SEQ ID: 130). In some aspects, the method comprises grafting the sFes¹ BC loop domain (GQSQPD (SEQ ID NO: 123)) into the BC loop of the SH2 domain to be modified. In some aspects, when the unmodified version of the SH2 domain contains a hydrophilic amino acid at position βC2 (position 70) and another hydrophilic amino acid at position βD6 (position 102), then the method further comprises replacing said hydrophilic amino acid at position βC2 and the hydrophilic amino acid at position βD6 with a hydrophobic amino acid, respectively. In some aspects, a Val residue is introduced into βC2 and an Ile residue is introduced at position βD6.

In some aspects, the sSH2 is sFes² (SEQ ID: 131). In some aspects, the method comprises grafting the sFes² BC loop domain (RQRKQE (SEQ ID NO: 124)) into the BC loop of the SH2 domain to be modified. In some aspects, when the unmodified version of the SH2 domain contains a hydrophilic amino acid at position βC2 (position 70) and another hydrophilic amino acid at position βD6 (position 102), the method further comprises replacing said hydrophilic amino acid at position βC2 and the hydrophilic amino acid at position βD6 with a hydrophobic amino acid, respectively. In some aspects, a Val residue is introduced into βC2 and an Ile residue is introduced at position βD6.

In some aspects, the sSH2 is sFes³ (SEQ ID: 132). In some aspects, the method comprises grafting the sFes³ BC loop domain (SPRIQE (SEQ ID NO: 125)) into the BC loop of the SH2 domain to be modified. In some aspects, when the unmodified version of the SH2 domain contains a hydrophilic amino acid at position βC2 (position 70) and another hydrophilic amino acid at position βD6 (position 102), the method further comprises replacing said hydrophilic amino acid at position βC2 and the hydrophilic amino acid at position βD6 with a hydrophobic amino acid, respectively. In some aspects, a Val residue is introduced into βC2 and an Ile residue is introduced at position βD6.

In some aspects, the sSH2 is sFes⁴ (SEQ ID: 133). In some aspects, the method comprises grafting the sFes⁴ BC loop domain (SQGRKV (SEQ ID NO: 126)) into the BC loop of the SH2 domain to be modified. In some aspects, when the unmodified version of the SH2 domain contains a hydrophilic amino acid at position βC2 (position 70) and another hydrophilic amino acid at position βD6 (position 102), the method further comprises replacing said hydrophilic amino acid at position βC2 and the hydrophilic amino acid at position βD6 with a hydrophobic amino acid, respectively. In some aspects, a Val residue is introduced into βC2 and an Ile residue is introduced at position βD6.

In some aspects, the sSH2 is sFes⁵ (SEQ ID: 134). In some aspects, the method comprises grafting the sFes⁵ BC loop domain (SISKQG (SEQ ID NO: 127)) into the BC loop of the SH2 domain to be modified. In some aspects, when the unmodified version of the SH2 domain contains a hydrophilic amino acid at position βC2 (position 70) and another hydrophilic amino acid at position βD6 (position 102), the method further comprises replacing said hydrophilic amino acid at position βC2 and the hydrophilic amino acid at position βD6 with a hydrophobic amino acid, respectively. In some aspects, a Val residue is introduced into βC2 and an Ile residue is introduced at position βD6.

In some aspects, the sSH2 is sFes⁶ (SEQ ID: 135). In some aspects, the method comprises grafting the sFes⁶ BC loop domain (SQTYPG (SEQ ID NO: 128)) into the BC loop of the SH2 domain to be modified. In some aspects, when the unmodified version of the SH2 domain contains a hydrophilic amino acid at position βC2 (position 70) and another hydrophilic amino acid at position βD6 (position 102), the method further comprises replacing said hydrophilic amino acid at position βC2 and the hydrophilic amino acid at position βD6 with a hydrophobic amino acid, respectively. In some aspects, a Val residue is introduced into βC2 and an Ile residue is introduced at position βD6.

In some aspects, the sSH2 is sSrc (SEQ ID: 136). In some aspects, the method comprises grafting the sSrc BC loop domain (SETVKGA (SEQ ID NO: 129)) into the BC loop of the SH2 domain to be modified. In some aspects, when the unmodified version of the SH2 domain contains a hydrophilic amino acid at position βC-2 (position 70) βD-6 (position 102), the method further comprises replacing said hydrophilic amino acids with a hydrophobic amino acid as said positions βC-2 and βD-6. In some aspects, an Ala is introduced into βC-2 (position 70) and a Leu is introduced into BD-6 (position 102).

ii. Arg Residue Grafting

In some instances, when an SH2 domain to be modified, following methods described herein, lacks an Arg residue in the αA-2 (position 33), then the method of creating modified SH2 domains disclosed here involves the substitution of the amino acid residue at position uA-2 (position 33) of the SH2 domain to be modified with an Arg residue.

As such, provided here is a method of producing a modified Src homology 2 (SH2) domain which exhibits equal or increased binding affinity to a pTyr-containing ligand relative to its unmodified version of the SH2 domain, the method comprising: substituting the amino acid residue at position αA-2 (position 33) of the SH2 domain to be modified with an Arg residue. In some aspects, the method further comprises replacing the BC loop of a parent SH2 domain with a BC loop of the SH2 domain superbinder described above. In some aspects, the method further comprises making one or more amino acid substitutions in the backside of the pTyr binding pocket in the SH2 domain fold of the parent SH2 domain (i.e., the SH2 domain to be modified), wherein the one or more amino acid substitutions provide hydrophobic interactions with pTyr.

### Phosphorylation Capturing Reagents

SuperSrc-SH2 has been used as an affinity purification tool in mass spectrometry (AP-MS) analyses to enrich the phosphoproteomes of cancer cell lines with unprecedented coverage, whereas superFyn-SH2 variants with altered specificity profiles enabled efficient enrichment of different phosphopeptides. Therefore, the SH2 superbinders with different specificity profiles provided herein can be used to probe the pTyr-phosphoproteome with greater depth and coverage than is possible with conventional anti-pTyr antibodies or natural SH2 domains that bind phosphopeptides with modest affinity.

Provided here is a protein phosphorylation capturing reagent comprising one or more peptides comprising the modified SH2 domain described herein. In some aspects, the one or more peptides are immobilized peptides. In some aspects, the one or more immobilized peptides are selected from Table 1; SEQ IDs: 130-180. In some aspects, the one or more immobilized peptides is immobilized to streptavidin beads. In some aspects, the immobilized peptides are biotinylated.

In some aspects, the modified SH2 disclosed herein is used alone (i.e. homogeneous mixture) or as a combination (i.e. a heterogeneous mixture of various SH2 variants, fused to one another using tags (e.g. Spy26/Snoop27 Tags or other similar technologies), or expressed as protein polymers in any combination) to make a universal SH2 superbinder affinity-purification tool. In some aspects, the superbinder affinity-purification tool disclosed herein covers a greater percentage of the pTyr phosphoproteome than conventional methods. A panel of 51 new SH2 variants (Table 1; SEQ IDs: 130-180) generated using phage display and/or the BC grafting method that can be used alone or in combination with one another to enrich pTyr containing peptides or proteins from samples of interest to analyze the pTyr peptide and/or proteins from these samples. Any SH2 variant produced using the method described above can also be used in the same manner to enrich or probe for pTyr containing peptides/proteins.

Also disclosed here are methods for isolating a pTyr-containing polypeptide from a complex mixture of peptides, the method comprising: (a) obtaining a proteinaceous preparation from an organism or bodily fluid; (b) contacting the preparation with the one or more immobilized polypeptides described herein; and (c) isolating polypeptides bound by the one or more immobilized peptides in step (b). In some aspects, the organisms are single cells, multicellular organisms (including subjects as this term is defined in this document), tissues, organs, or bacteria. In some aspects, the bodily fluids are blood (whole blood, blood plasma, blood serum, capillary blood, venous blood), tears, spinal fluid, synovial fluid, bronchoalveolar fluid, bronchoalveolar lavage, tissue extracts, urine, sweat, saliva, excrement, phlegm or other like bodily fluids excreted from organisms. In some aspects, the methods further comprise (d) characterizing the polypeptides isolated in step (c) by mass spectrometry (MS), tandem mass spectrometry (MS-MS), and/or MS3 analysis. In some aspects, the method further comprises (e) utilizing a search program to substantially match the spectra obtained for the polypeptides isolated in step (c) during the characterization of step (d) with the spectra for reference peptide sequences, thereby identifying parent proteins of the isolated polypeptide.

TABLE 1
Amino acid sequences of SH2 domains

| Identifier | SEQ ID: | Sequence |
|---|---|---|
| 3BP2 | 1 | VFVNTTESCEVERLFKATSPRGEPQDGLYCIRNSSTK |
| | | SGKVLVVWDETSNKVRNYRIFEKDSKFYLEGEVLFV |
| | | SVGSMVEHYHTHVLPSHQSLLLRHPY |
| Abl1 | 2 | WYHGPVSRNAAEYLLSSGINGSFLVRESESSPGQRSI |
| | | SLRYEGRVYHYRINTASDGKLYVSSESRFNTLAELV |
| | | HHHSTVADGLITTLHYPA |
| Ab12 | 3 | WYHGPVSRSAAEYLLSSLINGSFLVRESESSPGQLSIS |
| | | LRYEGRVYHYRINTTADGKVYVTAESRFSTLAELVH |
| | | HHSTVADGLVTTLHYPA |
| BCAR3 | 4 | WYHGRIPRQVSENLVQRDGDFLVRDSLSSPGNFVLT |
| | | CQWKNLAQHFKINRTVLRLSEAYSRVQYQFEMESFD |
| | | SIPGLVRCYVGNRRPISQQSGAIIFQPI |
| BLK | 5 | WFFRSQGRKEAERQLLAPINKAGSFLIRESETNKGAF |
| | | SLSVKDVTTQGELIKHYKIRCLDEGGYYISPRITFPSL |
| | | QALVQHYSKKGDGLCQRLTLPC |
| BLNK | 6 | WYAGACDRKSAEEALHRSNKDGSFLIRKSSGHDSKQ |
| | | PYTLVVFFNKRVYNIPVRFIEATKQYALGRKKNGEE |
| | | YFGSVAEIIRNHQHSPLVLIDSQNNTKDSTRLKYAV |
| BMX | 7 | WFAGNISRSQSEQLLRQKGKEGAFMVRNSSQVGMY |
| | | TVSLFSKAVNDKKGTVKHYHVHTNAENKLYLAENY |
| | | CFDSIPKLIHYHQHNSAGMITRLRHPV |
| BTK | 8 | WYSKHMTRSQAEQLLKQEGKEGGFIVRDSSKAGKY |
| | | TVSVFAKSTGDPQGVIRHYVVCSTPQSQYYLAEKHL |
| | | FSTIPELINYHQHNSAGLISRLKYPV |
| CBL | 9 | QPWSSLLRNWNSLAVTHPGYMAFLTYDEVKARLQK |
| | | FIHKPGSYIFRLSCTRLGQWAIGYVTADGNILQTIPHN |
| | | KPLFQALIDGFREGFYLFPDGRNQNPDLTG |
| CblB | 10 | QPWGSILRNWNFLAVTHPGYMAFLTYDEVKARLQK |
| | | YSTKPGSYIFRLSCTRLGQWAIGYVTGDGNILQTIPH |
| | | NKPLFQALIDGSREGFYLYPDGRSYNPDLTG |
| CblC | 11 | QPWPTLLKNWQLLAVNHPGYMAFLTYDEVQERLQ |
| | | ACRDKPGSYIFRPSCTRLGQWAIGYVSSDGSILQTIPA |
| | | NKPLSQVLLEGQKDGFYLYPDGKTHNPDLTE |
| CHIN | 12 | EFHGMISREAADQLLIVAEGSYLIRESQRQPGTYTLA |
| | | LRFGSQTRNFRLYYDGKHFVGEKRFESIHDLVTDGLI |
| | | TLYIETKAAEYIA |
| CHIO | 13 | EFHGIISREQADELLGGVEGAYILRESQRQPGCYTLA |
| | | LRFGNQTLNYRLFHDGKHFVGEKRFESIHDLV |
| CISH | 14 | WYWGSITASEARQHLQKMPEGTFLVRDSTHPSYLFT |
| | | LSVKTTRGPTNVRIEYADSSFRLDSNCLSRPRILAFPD |
| | | VVSLVQHY |
| CLNK | 15 | WYIGEYSRQAVEEAFMKENKDGSFLVRDCSTKSKEE |
| | | PYVLAVFYENKVYNVKIRFLERNQQFALGTGLRGDE |
| | | KFDSVEDIIEHYKNFPIILIDGKDKTGVHRKQCHLTQP |
| | | L |
| CRK | 16 | WYWGRLSRQEAVALLQGQRHGVFLVRDSSTSPGDY |
| | | VLSVSENSRVSHYIINSSGPRPPVPPSPAQPPPGVSPSR |
| | | LRIGDQEFDSLPALLEFYKIHYLDTTTLIEPV |
| CRKL | 17 | WYMGPVSRQEAQTRLQGQRHGMFLVRDSSTCPGDY |
| | | VLSVSENSRVSHYIINSLPNRRFKIGDQEFDHLPALLE |
| | | FYKIHYLDTTTLIEPA |
| CSK | 18 | WFHGKITREQAERLLYPPETGLFLVRESTNYPGDYTL |
| | | CVSCDGKVEHYRIMYHASKLSIDEEVYFENLMQLVE |
| | | HYTSDADGLCTRLIKPK |
| DAPP1 | 19 | WYHGNLTRHAAEALLLSNGCDGSYLLRDSNETTGL |
| | | YSLSVRAKDSVKHFHVEYTGYSFKFGFNEFSSLKDF |
| | | VKHFANQPLIGSETGTLMVLKHPY |
| Fer | 20 | WYHGAIPRIEAQELLKKQGDFLVRESHGKPGEYVLS |
| | | VYSDGQRRHFIIQYVDNMYRFEGTGFSNIPQLIDHHY |
| | | TTKQVITKKSGVVLLNPI |
| Fes | 21 | WYHGAIPRAEVAELLVHSGDFLVRESQGKQEYVLSV |
| | | LWDGLPRHFIIQSLDNLYRLEGEGFPSIPLLIDHLLST |
| | | QQPLTKKSGVVLHRAV |
| Fgr | 22 | WYFGKIGRKDAERQLLSPGNPQGAFLIRESETTKGA |
| | | YSLSIRDWDQTRGDHVKHYKIRKLDMGGYYITTRV |
| | | QFNSVQELVQHYMEVNDGLCNLLIAPC |
| FRK | 23 | WFFGAIGRSDAEKQLLYSENKTGSFLIRESESQKGEF |
| | | SLSVLDGAVVKHYRIKRLDEGGFFLTRRRIFSTLNEF |
| | | VSHYTKTSDGLCVKLGKPC |
| FYN | 24 | WYFGKLGRKDAERQLLSFGNPRGTFLIRESETTKGA |
| | | YSLSIRDWDDMKGDHVKHYKIRKLDNGGYYITTRA |
| | | QFETLQQLVQHYSERAAGLCCRLVVPC |
| GRAP | 25 | WYSGRISRQLAEEILMKRNHLGAFLIRESESSPGEFSV |
| | | SVNYGDQVQHFKVLREASGKYFLWEEKFNSLNELV |
| | | DFYRTTTIAKKRQIFLRDEEPL |
| GRAP2 | 26 | WFHEGLSRHQAENLLMGKEVGFFIIRASQSSPGDFSI |
| | | SVRHEDDVQHFKVMRDNKGNYFLWTEKFPSLNKLV |
| | | DYYRTNSISRQKQIFLRDRT |
| GRAPL | 27 | WYSGRISRQLAEEILMKRNHLGAFLIRESESSPGEFSV |
| | | SVNNRAQRGPCLGPKSHSRLG |
| Grb10 | 28 | WFHGRISREESHRIIKQQGLVDGLFLLRDSQSNPKAF |
| | | VLTLCHHQKIKNFQILPCEDDGQTFFSLDDGNTKFSD |
| | | LIQLVDFY |
| Grb14 | 29 | WFHHKISRDEAQRLIIQQGLVDGVFLVRDSQSNPKTF |
| | | VLSMSHGQKIKHFQIIPVEDDGEMFHTLDDGHTRFT |
| | | DLIQLVEFYQLNKGVLPCKLKHYC |
| Grb2 | 30 | WFFGKIPRAKAEEMLSKQRHDGAFLIRESESAPGDFS |
| | | LSVKFGNDVQHFKVLRDGAGKYFLWVVKFNSLNEL |
| | | VDYHRSTSVSRNQQIFLRDIE |
| Grb7/1-97 | 31 | WFHGRISREESQRLIGQQGLVDGLFLVRESQRNPQGF |
| | | VLSLCHLQKVKHYLILPSEEEGRLYFSMDDGQTRFT |
| | | DLLQLVEFHQLNRGILPCLLRHCC |
| HCK | 32 | WFFKGISRKDAERQLLAPGNMLGSFMIRDSETTKGS |
| | | YSLSVRDYDPRQGDTVKHYKIRTLDNGGFYISPRSTF |
| | | STLQELVDHYKKGNDGLCQKLSVPC |
| HSH2D | 33 | WFHGAISREDAENLLESQPLGSFLIRVSHSHVGYTLS |
| | | YKAQSSCCHFMVKLLDDGTFMIPGEKVAHTSLDAL |
| | | VTFHQQKPIEPRRELLTQPC |
| ITK | 34 | WYNKSISRDKAEKLLLDTGKEGAFMVRDSRTAGTY |
| | | TVSVFTKAVVSENNPCIKHYHIKETNDNPKRYYVAE |
| | | KYVFDSIPLLINYHQHNGGGLVTRLRYPV |
| JAK1 | 35 | GCHGPICTEYAINKLRQEGSEEGMYVLRWSCTDFDN |
| | | ILMTVTCFEKSEQVQGAQKQFKNFQIEVQKGRYSLH |
| | | GSDRSFPSLGDLMSHLKKQILRTDNISFMLKRCC |
| JAK2 | 36 | HGPISMDFAISKLKKAGNQTGLYVLRCSPKDFNKYF |
| | | LTFAVERENVIEYKHCLITKNENEEYNLSGTKKNFSS |
| | | LKDLLNCYQ |
| JAK3 | 37 | QCHGPITLDFAINKLKTGGSRPGSYVLRRSPQDFDSF |
| | | LLTVCVQNPLGPDYKGCLIRRSPTGTFLLVGLSRPHS |
| | | SLRELLATCWDGGLHVDGVAVTLTSCC |
| KSYK_N | 38 | FFFGNITREEAEDYLVQGGMSDGLYLLRQSRNYLGG |
| | | FALSVAHGRKAHHYTIERELNGTYAIAGGRTHASPA |
| | | DLCHYHSQESDGLVCLLKKPF |
| KYSK_C | 39 | WFHGKISREESEQIVLIGSKTNGKFLIRARDNNGSYA |
| | | LCLLHEGKVLHYRIDKDKTGKLSIPEGKKFDTLWQL |
| | | VEHYSYKADGLLRVLTVPC |
| Lck | 40 | WFFKNLSRKDAERQLLAPGNTHGSFLIRESESTAGSF |
| | | SLSVRDFDQNQGEVVKHYKIRNLDNGGFYISPRITFP |
| | | GLHELVRHYTNASDGLCTRLSRPC |
| LCP2 | 41 | WYVSYITRPEAEAALRKINQDGTFLVRDSSKKTTTNP |
| | | YVLMVLYKDKVYNIQIRYQKESQVYLLGTGLRGKE |
| | | DFLSVSDIIDYFRKMPLLLIDGKNRGSRYQCTLTHAA |
| LYN | 42 | WFFKDITRKDAERQLLAPGNSAGAFLIRESETLKGSF |
| | | SLSVRDFDPVHGDVIKHYKIRSLDNGGYYISPRITFPC |
| | | ISDMIKHYQKQADGLCRRLEKAC |
| MATK | 43 | WFHGKISGQEAVQQLQPPEDGLFLVRESARHPGDYV |
| | | LCVSFGRDVIHYRVLHRDGHLTIDEAVFFCNLMDMV |
| | | EHYSKDKGAICTKLVRPK |
| Nck1 | 44 | WYYGKVTRHQAEMALNERGHEGDFLIRDSESSPND |
| | | FSVSLKAQGKNKHFKVQLKETVYCIGQRKFSTMEEL |
| | | VEHYKKAPIFTSEQGEKLYLVKHL |
| Nck2 | 45 | WYYGNVTRHQAECALNERGVEGDFLIRDSESSPSDF |
| | | SVSLKASGKNKHFKVQLVDNVYCIGQRRFHTMDEL |
| | | VEHYKKAPIFTSEHGEKLYLVRAL |
| P55G_C | 46 | WFVEDINRVQAEDLLYGKPDGAFLIRESSKKGCYAC |
| | | SVVADGEVKHCVIYSTARGYGFAEPYNLYSSLKELV |
| | | LHYQQTSLVQHNDSLNVRLAYPV |
| P55G_N | 47 | WYWGDISREEVNDKLRDMPDGTFLVRDASTKMQG |
| | | DYTLTLRKGGNNKLIKIYHRDGKYGFSDPLTFNSVV |
| | | ELINHYHHESLAQYNPKLDVKLMYPV |
| P85A_C | 48 | WNVGSSNRNKAENLLRGKRDGTFLVRESSKQGCYA |
| | | CSVVVDGEVKHCVINKTATGYGFAEPYNLYSSLKEL |
| | | VLHYQHTSLVQHNDSLNVTLAYPV |
| P85A_N | 49 | WYWGDISREEVNEKLRDTADGTFLVRDASTKMHGD |
| | | YTLTLRKGGNNKLIKIFHRDGKYGFSDPLTFSSVVELI |
| | | NHYRNESLAQYNPKLDVKLLYPV |
| P85B_C | 50 | WYVGKINRTQAEEMLSGKRDGTFLIRESSQRGCYAC |
| | | SVVVDGDTKHCVIYRTATGFGFAEPYNLYGSLKELV |
| | | LHYQHASLVQHNDALTVTLAHPV |
| P85B_N | 51 | WYWGDISREEVNEKLRDTPDGTFLVRDASSKIQGEY |
| | | TLTLRKGGNNKLIKVFHRDGHYGFSEPLTFCSVVDLI |
| | | NHYRHESLAQYNAKLDTRLLYPV |
| PLCGI_C | 52 | WYHASLTRAQAEHMLMRVPRDGAFLVRKRNEPNS |
| | | YAISFRAEGKIKHCRVQQEGQTVMLGNSEFDSLVDLI |
| | | SYYEKHPLYRKMKLRYPI |
| PLCGI_N | 53 | WFHGKLGAGRDGRHIAERLLTEYCIETGAPDGSFLV |
| | | RESETFVGDYTLSFWRNGKVQHCRIHSRQDAGTPKF |
| | | FLTDNLVFDSLYDLITHYQQVPLRCNEFEMRLSEPV |
| PLCG2_C | 54 | WYYDSLSRGEAEDMLMRIPRDGAFLIRKREGSDSYA |
| | | ITFRARGKVKHCRINRDGRHFVLGTSAYFESLVELVS |
| | | YYEKHSLYRKMRLRYPV |
| PLCG2_N | 55 | WFHKKVEKRTSAEKLLQEYCMETGGKDGTFLVRES |
| | | ETFPNDYTLSFWRSGRVQHCRIRSTMEGGTLKYYLT |
| | | DNLTFSSIYALIQHYRETHLRCAEFELRLTDPV |
| PTK6 | 56 | WFFGCISRSEAVRRLQAEGNATGAFLIRVSEKPSADY |
| | | VLSVRDTQAVRHYKIWRRAGGRLHLNEAVSFLSLPE |
| | | LVNYHRAQSLSHGLRLAAPC |
| PTN11_C | 57 | WFHGHLSGKEAEKLLTEKGKHGSFLVRESQSHPGDF |
| | | VLSVRTGDDKGESNDGKSKVTHVMIRCQELKYDVG |
| | | GGERFDSLTDLVEHYKKNPMVETLGTVLQLKQPL |
| PTN11_N | 58 | WFHPNITGVEAENLLLTRGVDGSFLARPSKSNPGDFT |
| | | LSVRRNGAVTHIKIQNTGDYYDLYGGEKFATLAELV |
| | | QYYMEHHGQLKEKNGDVIELKYPL |
| PTN6_C | 59 | WYHGHMSGGQAETLLQAKGEPWTFLVRESLSQPGD |
| | | FVLSVLSDQPKAGPGSPLRVTHIKVMCEGGRYTVGG |
| | | LETFDSLTDLVEHFKKTGIEEASGAFVYLRQPY |
| PTN6_N | 60 | WFHRDLSGLDAETLLKGRGVHGSFLARPSRKNQGD |
| | | FSLSVRVGDQVTHIRIQNSGDFYDLYGGEKFATLTEL |
| | | VEYYTQQQGVLQDRDGTIIHLKYPL |
| RASAI_C | 61 | WFHGKISKQEAYNLLMTVGQVCSFLVRPSDNTPGD |
| | | YSLYFRTNENIQRFKICPTPNNQFMMGGRYYNSIGDII |
| | | DHYRKEQIVEGYYLKEPV |
| RASAI_N | 62 | WYHGKLDRTIAEERLRQAGKSGSYLIRESDRRPGSF |
| | | VLSFLSQMNVVNHFRIIAMCGDYYIGGRRFSSLSDLI |
| | | GYYSHVSCLLKGEKLLYPV |
| RIN1 | 63 | WLQLQANAAAALHMLRTEPPGTFLVRKSNTRQCQA |
| | | LCMRLPEASGPSFVSSHYILESPGGVSLEGSELMFPDL |
| | | VQLICAYCHTRDILLLPLQLPR |
| RIN2 | 64 | WLQLSLSEEEAAEVLQAQPPGIFLVHKSTKMQKKVL |
| | | SLRLPCEFGAPLKEFAIKESTYTFSLEGSGISFADLFRL |
| | | IAFYCISRDVLPFTLKLPY |
| RIN3/1-96 | 65 | WLQLSLGQAEVARILHRVVAGMFLVRRDSSSKQLV |
| | | LCVHFPSLNESSAEVLEYTIKEEKSILYLEGSALVFED |
| | | IFRLIAFYCVSRDLLPFTLRLPQ |
| SH21A | 66 | VYHGKISRETGEKLLLATGLDGSYLLRDSESVPGVY |
| | | CLCVLYHGYIYTYRVSQTETGSWSAETAPGVHKRYF |
| | | RKIKNLISAFQKPDQGIVIPLQYPVEK |
| SH21B | 67 | YYHGRLTKQDCETLLLKEGVDGNFLLRDSESIPGVL |
| | | CLCVSFKNIVYTYRIFREKHGYYRIQTAEGSPKQVFP |
| | | SLKELISKFEKPNQGMVVHLLKPI |
| SH22A | 68 | WFHGFITRREAERLLEPKPQGCYLVRFSESAVTFVLT |
| | | YRSRTCCRHFLLAQLRDGRHVVLGEDSAHARLQDL |
| | | LLHYTAHPLSPYGETLTEPL |
| SH23 | 69 | AWYHGLLSRQKAEALLQQNGDFLVRASGSRGGNPV |
| | | ISCRWRGSALHFEVFRVALRPRPGRPTALFQLEDEQF |
| | | PSIPALVHSYMTGRRPLSQATGAVVSRPV |
| SH24A | 70 | WFHGILTLKKANELLLSTGMPGSFLIRVSERIKGYAL |
| | | SYLSEDGCKHFLIDASADAYSFLGVDQLQHATLADL |
| | | VEYHKEEPITSLGKELLLYPC |
| SH24B | 71 | WFHGIISREDAEALLENMTEGAFLVRVSEKIWGYTLS |
| | | YRLQKGFKHFLVDASGDFYSFLGVDPNRHATLTDLV |
| | | DFHKEEIITVSGGELLQEPC |
| SH2B1 | 72 | WFHGMLSRLKAAQLVLTGGTGSHGVFLVRQSETRR |
| | | GEYVLTFNFQGKAKHLRLSLNEEGQCRVQHLWFQSI |
| | | FDMLEHFRVHPIPLESGGSSDVVLVSYV |
| SH2B2 | 73 | WFHGTLSRVKAAQLVLAGGPRNHGLFVIRQSETRPG |
| | | EYVLTFNFQGKAKHLRLSLNGHGQCHVQHLWFQSV |
| | | LDMLRHFHTHPIPLESGGSADITLRSYV |
| SH2B3 | 74 | WFHGPISRVKAAQLVQLQGPDAHGVFLVRQSETRR |
| | | GEYVLTFNFQGIAKHLRLSLTERGQCRVQHLHFPSV |
| | | VDMLHHFQRSPIPLECGAACDVRLSSYV |
| SH2D3 | 75 | WYHGRIPREVSETLVQRNGDFLIRDSLTSLGDYVLTC |
| | | RWRNQALHFKINKVVVKAGESYTHIQYLFEQESFDH |
| | | VPALVRYHVGSRKAVSEQSGAIIYCPV |
| SH2D5 | 76 | WAFAGISRPCALALLRRDVLGAFLLWPELGASGQW |
| | | CLSVRTQCGVVPHQVFRNHLGRYCLEHLPAEFPSLE |
| | | ALVENHAVTERSLFCPLDMGRLNPTY |
| SH2D6 | 77 | WYSGNCDRYAVESALLHLQKDGAYTVRPSSGPHGS |
| | | QPFTLAVLLRGRVFNIPIRRLDGGRHYALGREGRNRE |
| | | ELFSSVAAMVQHFMWHPLPLVDRHSGSRELTCLLFP |
| | | T |
| SH2D7 | 78 | WFHGFITRKQTEQLLRDKALGSFLIRLSDRATGYILS |
| | | YRGSDRCRHFVINQLRNRRYIISGDTQSHSTLAELVH |
| | | HYQEAQLEPFKEMLTAAC |
| SHB | 79 | WYHGAISRGDAENLLRLCKECSYLVRNSQTSKHDYS |
| | | LSLRSNQGFMHMKLAKTKEKYVLGQNSPPFDSVPEV |
| | | IHYYTTRKLPIKGAEHLSLLYPV |
| SHC1 | 80 | WFHGKLSRREAEALLQLNGDFLVRESTTTPGQYVLT |
| | | GLQSGQPKHLLLVDPEGVVRTKDHRFESVSHLISYH |
| | | MDNHLPIISAGSELCLQQPV |
| SHC2 | 81 | WYHGRMSRRAAERMLRADGDFLVRDSVTNPGQYV |
| | | LTGMHAGQPKHLLLVDPEGVVRTKDVLFESISHLID |
| | | HHLQNGQPIVAAESELHLRGVV |
| SHC3 | 82 | WYQGEMSRKEAEGLLEKDGDFLVRKSTTNPGSFVL |
| | | TGMHNGQAKHLLLVDPEGTIRTKDRVFDSISHLINH |
| | | HLESSLPIVSAGSELCLQQPV |
| SHC4 | 83 | CYHGKLSRKAAESLLVKDGDFLVRESATSPGQYVLS |
| | | GLQGGQAKHLLLVDPEGKVRTKDHVFDNVGHLIRY |
| | | HMDNSLPIISSGSEVSLKQPV |
| SHD | 84 | WFHGPLNRADAESLLSLCKEGSYLVRLSETNPQDCS |
| | | LSLRSSQGFLHLKFARTRENQVVLGQHSGPFPSVPEL |
| | | VLHYSSRPLPVQGAEHLALLYPV |
| SHE | 85 | WYHGAISRAEAESRLQPCKEAGYLVRNSESGNSRYS |
| | | IALKTSQGCVHIIVAQTKDNKYTLNQTSAVFDSIPEV |
| | | VHYYSNEKLPFKGAEHMTLLYPV |
| SHF | 86 | WYHGAISRTDAENLLRLCKEASYLVRNSETSKNDFS |
| | | LSLKSSQGFMHMKLSRTKEHKYVLGQNSPPFSSVPEI |
| | | VHHYASRKLPIKGAEHMSLLYPV |
| SHIP1 | 87 | WNHGNITRSKAEELLSRTGKDGSFLVRASESISRAYA |
| | | LCVLYRNCVYTYRILPNEDDKFTVQASEGVSMRFFT |
| | | KLDQLIEFYKKENMGLVTHLQYPV |
| SHIP2 | 88 | WYHRDLSRAAAEELLARAGRDGSFLVRDSESVAGA |
| | | FALCVLYQKHVHTYRILPDGEDFLAVQTSQGVPVRR |
| | | FQTLGELIGLYAQPNQGLVCALLLPV |
| SLAP1 | 89 | WLFEGLGRDKAEELLQLPDTKVGSFMIRESETKKGF |
| | | YSLSVRHRQVKHYRIFRLPNNWYYISPRLTFQCLEDL |
| | | VNHYSEVADGLCCVLTTPC |
| SLAP2 | 90 | WLYEGLSREKAEELLLLPGNPGGAFLIRESQTRRGSY |
| | | SLSVRLSRPASWDRIRHYRIHCLDNGWLYISPRLTFPS |
| | | LQALVDHYSELADDICCLLKEPC |
| SOCS1 | 91 | FYWGPLSVHGAHERLRAEPVGTFLVRDSRQRNCFFA |
| | | LSVKMASGPTSIRVHFQAGRFHLDGSRESFDCLFELL |
| | | EHYVAAPRRMLGAPLRQRRVRP |
| SOCS2 | 92 | WYWGSMTVNEAKEKLKEAPEGTFLIRDSSHSDYLLT |
| | | ISVKTSAGPTNLRIEYQDGKFRLDSIICVKSKLKQFDS |
| | | VVHLIDYYVQMCKDKRTGPEAPRNGTVHLYLTKPL |
| SOCS3 | 93 | FYWSAVTGGEANLLLSAEPAGTFLIRDSSDQRHFFTL |
| | | SVKTQSGTKNLRIQCEGGSFSLQSDPRSTQPVPRFDC |
| | | VLKLVHHYMPPPGAPSFPSPPTE |
| SOCS4 | 94 | CYWGVMDKYAAEALLEGKPEGTFLLRDSAQEDYLF |
| | | SVSFRRYSRSLHARIEQWNHNFSFDAHDPCVFHSPDI |
| | | TGLLEHYKDPSACMFFEPLLSTPL |
| SOCS5 | 95 | CYWGVMDRYEAEALLEGKPEGTFLLRDSAQEDYLF |
| | | SVSFRRYNRSLHARIEQWNHNFSFDAHDPCVFHSST |
| | | VTGLLEHYKDPSSCMFFEPLLTISL |
| SOCS6 | 96 | WYWGPITRWEAEGKLANVPDGSFLVRDSSDDRYLL |
| | | SLSFRSHGKTLHTRIEHSNGRFSFYEQPDVEGHTSIVD |
| | | LIEHSIRDSENGAFCYSRSRLPGS |
| SOCS7 | 97 | WYWGPMNWEDAEMKLKGKPDGSFLVRDSSDPRYI |
| | | LSLSFRSQGITHHTRMEHYRGTFSLWCHPKFEDRCQS |
| | | VVEFIKRAIMHSKNGKFLYFLRSRVPGL |
| Spt6H | 98 | YIKRVIAHPSFHNINFKQAEKMMETMDQGDVIIRPSS |
| | | KGENHLTVTWKVSDGIYQHVDVREEGKENAFSLGA |
| | | TLWINSEEFEDLDEIVARYVQPMASFARDLLNHKY |
| Src | 99 | WYFGKITRRESERLLLNAENPRGTFLVRESETTKGAY |
| | | CLSVSDFDNAKGLNVKHYKIRKLDSGGFYITSRTQF |
| | | NSLQQLVAYYSKHADGLCHRLTTVC |
| SRMS | 100 | WYFSGVSRTQAQQLLLSPPNEPGAFLIRPSESSLGGY |
| | | SLSVRAQAKVCHYRVSMAADGSLYLQKGRLFPGLE |
| | | ELLTYYKANWKLIQNPLLQPC |
| STAP1 | 101 | ACFYTVSRKEATEMLQKNPSLGNMILRPGSDSRNYSI |
| | | TIRQEIDIPRIKHYKVMSVGQNYTIELEKPVTLPNLFS |
| | | VIDYFVKETRGNLRPFICSTDENTGQEPS |
| STAP2 | 102 | YMMSEVLAKEEARRALETPSCFLKVSRLEAQLLLER |
| | | YPECGNLLLRPSGDGADGVSVTTRQMHNGTHVVRH |
| | | YKVKREGPKYVIDVEQPFSCTSLDAVVNYFVSHTKK |
| | | ALVPFLLDE |
| STATI | 103 | WNDGCIMGFISKERERALLKDQQPGTFLLRFSESSRE |
| | | GAITFTWVERSQNGGEPDFHAVEPYTKKELSAVTFP |
| | | DIIRNYKVMAAENIPENPLKYLYPN |
| STAT2 | 104 | WNDGRIMGFVSRSQERRLLKKTMSGTFLLRFSESSE |
| | | GGITCSWVEHQDDDKVLIYSVQPYTKEVLQSLPLTEI |
| | | IRHYQLLTEENIPENPLRFLYPR |
| STAT3 | 105 | WNEGYIMGFISKERERAILSTKPPGTFLLRFSESSKEG |
| | | GVTFTWVEKDISGKTQIQSVEPYTKQQLNNMSFAEII |
| | | MGYKIMDATNILVSPL |
| STAT4 | 106 | WIDGYVMGFVSKEKERLLLKDKMPGTFLLRFSESHL |
| | | GGITFTWVDHSESGEVRFHSVEPYNKGRLSALPFADI |
| | | LRDYKVIMAENIPENPLKYLYPD |
| STAT5A | 107 | WNDGAILGFVNKQQAHDLLINKPDGTFLLRFSDSEIG |
| | | GITIAWKFDSPERNLWNLKPFTTRDFSIRSLADRLGD |
| | | LSYLIYVFPDRPKDEVFSKYYTPV |
| STAT5B | 108 | WNDGAILGFVNKQQAHDLLINKPDGTFLLRFSDSEIG |
| | | GITIAWKFDSQERMFWNLMPFTTRDFSIRSLADRLGD |
| | | LNYLIYVFPDRPKDEVYSKYYTPV |
| STAT6 | 109 | WFDGVLDLTKRCLRSYWSDRLIIGFISKQYVTSLLLN |
| | | EPDGTFLLRFSDSEIGGITIAHVIRGQDGSPQIENIQ |
| | | PFSAKDLSIRSLGDRIRDLAQLKNLYPKKPKDEAFRS |
| | | HYKPE |
| TEC | 110 | WYCRNMNRSKAEQLLRSEDKEGGFMVRDSSQPGLY |
| | | TVSLYTKFGGEGSSGFRHYHIKETTTSPKKYYLAEKH |
| | | AFGSIPEIIEYHKHNAAGLVTRLRYPV |
| TENS1 | 111 | WYKPEISREQAIALLKDQEPGAFIIRDSHSFRGAYGL |
| | | AMKVSSPPPTIMQQNKKGDMTHELVRHFLIETGPRG |
| | | VKLKGCPNEPNFGSLSALVYQHSIIPLALPCKLVIPN |
| TENS3 | 112 | WYKADISREQAIAMLKDKEPGSFIVRDSHSFRGAYG |
| | | LAMKVATPPPSVLQLNKKAGDLANELVRHFLIECTP |
| | | KGVRLKGCSNEPYFGSLTALVCQHSITPLALPCKLL |
| | | IPE |
| TENS4 | 113 | WFKPNITREQAIELLRKEEPGAFVIRDSSSYRGSFGLA |
| | | LKVQEVPASAQSRPGEDSNDLIRHFLIESSAKGVHLK |
| | | GADEEPYFGSLSAFVCQHSIMALALPCKLTIPQ |
| TNS2 | 114 | WYKPHLSRDQAIALLKDKDPGAFLIRDSHSFQGAYG |
| | | LALKVATPPPSAQPWKGDPVEQLVRHFLIETGPKGV |
| | | KIKGCPSEPYFGSLSALVSQHSISPISLPCCLRIPS |
| TXK | 115 | WYHRNITRNQAEHLLRQESKEGAFIVRDSRHLGSYTI |
| | | SVFMGARRSTEAAIKHYQIKKNDSGQWYVAERHAF |
| | | QSIPELIWYHQHNAAGLMTRLRYPV |
| Tyk2 | 116 | GIHGPLLEPFVQAKLRPEDGLYLIHWSTSHPYRLILTV |
| | | AQRSQAPDGMQSLRLRKFPIEQQDGAFVLEGWGRSF |
| | | PSVREL |
| VAV | 117 | WYAGPMERAGAESILANRSDGTFLVRQRVKDAAEF |
| | | AISIKYNVEVKHIKIMTAEGLYRITEKKAFRGLTELVE |
| | | FYQQNSLKDCFKSLDTTLQFPF |
| VAV2 | 118 | WFAGNMERQQTDNLLKSHASGTYLIRERPAEAERFA |
| | | ISIKFNDEVKHIKVVEKDNWIHITEAKKFDSLLELVE |
| | | YYQCHSLKESFKQLDTTLKYPY |
| VAV3 | 119 | WYAGAMERLQAETELINRVNSTYLVRHRTKESGEY |
| | | AISIKYNNEAKHIKILTRDGFFHIAENRKFKSLMELVE |
| | | YYKHHSLKEGFRTLDTTLQFPY |
| YES | 120 | WYFGKMGRKDAERLLLNPGNQRGIFLVRESETTKG |
| | | AYSLSIRDWDEIRGDNVKHYKIRKLDNGGYYITTRA |
| | | QFDTLQKLVKHYTEHADGLCHKLTTVC |
| ZAP70_C | 121 | WYHSSLTREEAERKLYSGAQTDGKFLLRPRKEQGTY |
| | | ALSLIYGKTVYHYLISQDKAGKYCIPEGTKFDTLWQ |
| | | LVEYLKLKADGLIYCLKEAC |
| ZAP70_N | 122 | FFYGSISRAEAEEHLKLAGMADGLFLLRQCLRSLGG |
| | | YVLSLVHDVRFHHFPIERQLNGTYAIAGGKAHCGPA |
| | | ELCEFYSRDPDGLPCNLRKPC |
| sFes¹ BC Loop | 123 | GQSQPD |
| sFes² BC Loop | 124 | RQRKQE |
| sFes³ BC Loop | 125 | SPRIQE |
| sFes⁴ BC Loop | 126 | SQGRKV |
| sFes⁵ BC Loop | 127 | SISKQG |
| sFes⁶ BC Loop | 128 | SQTYPG |
| sSrc BC Loop | 129 | SETVKGA |
| sFes¹ (SuperFes) | 130 | MKIEEHHHHHHSSGKLGLNDIFEAQKIEWHESSGED |
| | | LYFQSGTTMDDYKDDDDKGGSEVQKPLHEQLWYH |
| | | GAIPRAEVAELLVHSGDFLVREGQSQPDYVLSVLWD |
| | | GLPRHFIIQSLDNLYRLEGEGFPSIPLLIDHLLSTQQPL |
| | | TKKSGVVLHRAVPSR |
| sFes² | 131 | MKIEEHHHHHHSSGKLGLNDIFEAQKIEWHESSGED |
| | | LYFQSGTTMDDYKDDDDKGGSEVQKPLHEQLWYH |
| | | GAIPRAEVAELLVHSGDFLVRERQRKQEYVLSVLWD |
| | | GLPRHFIIQSLDNLYRLEGEGFPSIPLLIDHLLSTQQPL |
| | | TKKSGVVLHRAVPSR |
| sFes³ | 132 | MKIEEHHHHHHSSGKLGLNDIFEAQKIEWHESSGED |
| | | LYFQSGTTMDDYKDDDDKGGSEVQKPLHEQLWYH |
| | | GAIPRAEVAELLVHSGDFLVRESPRIQEYVLSVLWDG |
| | | LPRHFIIQSLDNLYRLEGEGFPSIPLLIDHLLSTQQPLT |
| | | KKSGVVLHRAVPSR |
| sFes⁴ | 133 | MKIEEHHHHHHSSGKLGLNDIFEAQKIEWHESSGED |
| | | LYFQSGTTMDDYKDDDDKGGSEVQKPLHEQLWYH |
| | | GAIPRAEVAELLVHSGDFLVRESQGRKVYVLSVLWD |
| | | GLPRHFIIQSLDNLYRLEGEGFPSIPLLIDHLLSTQQPL |
| | | TKKSGVVLHRAVPSR |
| sFes⁵ | 134 | MKIEEHHHHHHSSGKLGLNDIFEAQKIEWHESSGED |
| | | LYFQSGTTMDDYKDDDDKGGSEVQKPLHEQLWYH |
| | | GAIPRAEVAELLVHSGDFLVRESISKQGYVLSVLWD |
| | | GLPRHFLIQSLDNLYRLEGEGFPSIPLLIDHLLSTQQPL |
| | | TKKSGVVLHRAVPSR |
| sFes⁶ | 135 | MKIEEHHHHHHSSGKLGLNDIFEAQKIEWHESSGED |
| | | LYFQSGTTMDDYKDDDDKGGSEVQKPLHEQLWYH |
| | | GAIPRAEVAELLVHSGDFIVRESQTYPGYVLSVLWD |
| | | GLPRHFIIQSLDNLYRLEGEGFPSIPLLIDHLLSTQQPL |
| | | TKKSGVVLHRAVPSR |
| sSrc (superSrc) | 136 | MKIEEHHHHHHSSGKLGLNDIFEAQKIEWHESSGED |
| | | LYFQSGTTMDDYKDDDDKGGSDSIQAEEWYFGKIT |
| | | RRESERLLLNAENPRGTFLVRESETVKGAYALSVSDF |
| | | DNAKGLNVKHYLIRKLDSGGFYITSRTQFNSLQQLV |
| | | AYYSKHADGLCHRLTTVCPSR |
| Abl1^{sFes} | 137 | MKIEEHHHHHHSSGKLGLNDIFEAQKIEWHESSGED |
| | | LYFQSGTTMDDYKDDDDKGGSSPSNYITPVNSLEKH |
| | | SWYHGPVSRNAAEYLLSSGINGSFLVREGQSQPDRVI |
| | | SLRYEGRVYHYIINTASDGKLYVSSESRFNTLAELVH |
| | | HHSTVADGLITTLHYPAPKRNKPTVYGVSPNYSR |
| Abl1^{sSrc} | 138 | MKIEEHHHHHHSSGKLGLNDIFEAQKIEWHESSGED |
| | | LYFQSGTTMDDYKDDDDKGGSSPSNYITPVNSLEKH |
| | | SWYHGPVSRNAAEYLLSSGINGSFLVRESETVKGAR |
| | | AISLRYEGRVYHYLINTASDGKLYVSSESRENTLAEL |
| | | VHHHSTVADGLITTLHYPAPKRNKPTVYGVSPNYSR |
| BLK^{sFes} | 139 | MKIEEHHHHHHSSGKLGLNDIFEAQKIEWHESSGED |
| | | LYFQSGTTMDDYKDDDDKGSARVESLEMERWFFRS |
| | | QGRKEAERQLLAPINKAGSFLIREGQSQPDFVLSVKD |
| | | VTTQGELIKHYIIRCLDEGGYYISPRITFPSLQALVQH |
| | | YSKKGDGLCQRLTLPCVRPAPQNPWSR |
| BLK^{sSrc} | 140 | MKIEEHHHHHHSSGKLGLNDIFEAQKIEWHESSGED |
| | | LYFQSGTTMDDYKDDDDKGSARVESLEMERWFFRS |
| | | QGRKEAERQLLAPINKAGSFLIRESETVKGAFALSVK |
| | | DVTTQGELIKHYLIRCLDEGGYYISPRITFPSLQALVQ |
| | | HYSKKGDGLCQRLTLPCVRPAPQNPWSR |
| BTK^{sFes} | 141 | MKIEEHHHHHHSSGKLGLNDIFEAQKIEWHESSGED |
| | | LYFQSGTTMDDYKDDDDKGSEAEDSIEMYEWYSKH |
| | | MTRSQAEQLLKQEGKEGGFIVRDGQSQPDYVVSVFA |
| | | KSTGDPQGVIRHYIVCSTPQSQYYLAEKHLFSTIPE |
| | | LINYHQHNSAGLISRLKYPVSQQNKNAPSSR |
| BTK^{sSrc} | 142 | MKIEEHHHHHHSSGKLGLNDIFEAQKIEWHESSGED |
| | | LYFQSGTTMDDYKDDDDKGSEAEDSIEMYEWYSKH |
| | | MTRSQAEQLLKQEGKEGGFIVRDSETVKGAYAVSVF |
| | | AKSTGDPQGVIRHYLVCSTPQSQYYLAEKHLFSTIPE |
| | | LINYHQHNSAGLISRLKYPVSQQNKNAPSSR |
| CRKL^{sFes} | 143 | MKIEEHHHHHHSSGKLGLNDIFEAQKIEWHESSGED |
| | | LYFQSGTTMDDYKDDDDKGSARFDSSDRSAWYMG |
| | | PVSRQEAQTRLQGQRHGMFLVRDGQSQPDYVLSVS |
| | | ENSRVSHYIINSLPNRRFKIGDQEFDHLPALLEFY |
| | | KIHYLDTTTLIEPAPRYPSPPMGSR |
| CRKL^{sSrc} | 144 | MKIEEHHHHHHSSGKLGLNDIFEAQKIEWHESSGED |
| | | LYFQSGTTMDDYKDDDDKGSARFDSSDRSAWYMG |
| | | PVSRQEAQTRLQGQRHGMFLVRDSETVKGAYALSV |
| | | SENSRVSHYLINSLPNRRFKIGDQEFDHLPALLEFYKI |
| | | HYLDTTTLIEPAPRYPSPPMGSR |
| Fes^{sSrc} | 145 | MKIEEHHHHHHSSGKLGLNDIFEAQKIEWHESSGED |
| | | LYFQSGTTMDDYKDDDDKGGSEVQKPLHEQLWYH |
| | | GAIPRAEVAELLVHSGDFLVRESETVKGAYALSVLW |
| | | DGLPRHFLIQSLDNLYRLEGEGFPSIPLLIDHLLSTQQ |
| | | PLTKKSGVVLHRAVPSR |
| Grb2^{sFes} | 146 | MKIEEHHHHHHSSGKLGLNDIFEAQKIEWHESSGED |
| | | LYFQSGTTMDDYKDDDDKGGSPKNYIEMKPHPWFF |
| | | GKIPRAKAEEMLSKQRHDGAFLIREGQSQPDFVLSV |
| | | KFGNDVQHFIVLRDGAGKYFLWVVKFNSLNELVDY |
| | | HRSTSVSRNQQIFLRDIEQVPQQPTYVQASR |
| Grb2^{sSrc} | 147 | MKIEEHHHHHHSSGKLGLNDIFEAQKIEWHESSGED |
| | | LYFQSGTTMDDYKDDDDKGGSPKNYIEMKPHPWFF |
| | | GKIPRAKAEEMLSKQRHDGAFLIRESETVKGAFALS |
| | | VKFGNDVQHFLVLRDGAGKYFLWVVKFNSLNELVD |
| | | YHRSTSVSRNQQIFLRDIEQVPQQPTYVQASRYNPVI |
| | | LIMKRWHRYN |
| LCK^{sFes} | 148 | MKIEEHHHHHHSSGKLGLNDIFEAQKIEWHESSGED |
| | | LYFQSGTTMDDYKDDDDKGSAKANSLEPEPWFFKN |
| | | LSRKDAERQLLAPGNTHGSFLIREGQSQPDFVLSVRD |
| | | FDQNQGEVVKHYIIRNLDNGGFYISPRITFPGLHELV |
| | | RHYTNASDGLCTRLSRPCQTQKPQKPWSR |
| LCK^{sSrc} | 149 | MKIEEHHHHHHSSGKLGLNDIFEAQKIEWHESSGED |
| | | LYFQSGTTMDDYKDDDDKGSAKANSLEPEPWFFKN |
| | | LSRKDAERQLLAPGNTHGSFLIRESETVKGAFALSVR |
| | | DFDQNQGEVVKHYLIRNLDNGGFYISPRITFPGLHEL |
| | | VRHYTNASDGLCTRLSRPCQTQKPQKPWSR |
| NCK1^{sFes} | 150 | MKIEEHHHHHHSSGKLGLNDIFEAQKIEWHESSGED |
| | | LYFQSGTTMDDYKDDDDKGSSLTGKFAGNPWYYG |
| | | KVTRHQAEMALNERGHEGDFLIRDGQSQPDFVVSLK |
| | | AQGKNKHFIVQLKETVYCIGQRKFSTMEELVEHYKK |
| | | APIFTSEQGEKLYLVKHLSSR |
| NCK1^{sSrc} | 151 | MKIEEHHHHHHSSGKLGLNDIFEAQKIEWHESSGED |
| | | LYFQSGTTMDDYKDDDDKGSSLTGKFAGNPWYYG |
| | | KVTRHQAEMALNERGHEGDFLIRDSETVKGAFAVSL |
| | | KAQGKNKHFLVQLKETVYCIGQRKFSTMEELVEHY |
| | | KKAPIFTSEQGEKLYLVKHLSSR |
| P55G_N^{sFes} | 152 | MKIEEHHHHHHSSGKLGLNDIFEAQKIEWHESSGED |
| | | LYFQSGTTMDDYKDDDDKGSDSSVSLQDAEWYWG |
| | | DISREEVNDKLRDMPDGTFLVRDGQSQPDYVLTLRK |
| | | GGNNKLIIIYHRDGKYGFSDPLTFNSVVELINHYHHE |
| | | SLAQYNPKLDVKLMYPVSRYQQDQLVSR |
| P55G_N^{sSrc} | 153 | MKIEEHHHHHHSSGKLGLNDIFEAQKIEWHESSGED |
| | | LYFQSGTTMDDYKDDDDKGSDSSVSLQDAEWYWG |
| | | DISREEVNDKLRDMPDGTFLVRDSETVKGAYALTLR |
| | | KGGNNKLILIYHRDGKYGFSDPLTFNSVVELINHYHH |
| | | ESLAQYNPKLDVKLMYPVSRYQQDQLVSR |
| P85A_N^{sFes} | 154 | MKIEEHHHHHHSSGKLGLNDIFEAQKIEWHESSGED |
| | | LYFQSGTTMDDYKDDDDKGGSSLQNAEWYWGDIS |
| | | REEVNEKLRDTADGTFLVRDGQSQPDYVLTLRKGG |
| | | NNKLIIIFHRDGKYGFSDPLTFSSVVELINHYRNESLA |
| | | QYNPKLDVKLLYPVSKSR |
| P85A_N^{sSre} | 155 | MKIEEHHHHHHSSGKLGLNDIFEAQKIEWHESSGED |
| | | LYFQSGTTMDDYKDDDDKGGSSLQNAEWYWGDIS |
| | | REEVNEKLRDTADGTFLVRDSETVKGAYALTLRKG |
| | | GNNKLILIFHRDGKYGFSDPLTFSSVVELINHYRNESL |
| | | AQYNPKLDVKLLYPVSKSR |
| P85B_N^{sFes} | 156 | MKIEEHHHHHHSSGKLGLNDIFEAQKIEWHESSGED |
| | | LYFQSGTTMDDYKDDDDKGSGSPPSLQDAEWYWG |
| | | DISREEVNEKLRDTPDGTFLVRDGQSQPDYVLTLRK |
| | | GGNNKLIIVFHRDGHYGFSEPLTFCSVVDLINHYRHE |
| | | SLAQYNAKLDTRLLYPVSRYQQDQIVSR |
| P85B_N^{sSrc} | 157 | MKIEEHHHHHHSSGKLGLNDIFEAQKIEWHESSGED |
| | | LYFQSGTTMDDYKDDDDKGSGSPPSLQDAEWYWG |
| | | DISREEVNEKLRDTPDGTFLVRDSETVKGAYALTLR |
| | | KGGNNKLILVFHRDGHYGFSEPLTFCSVVDLINHYR |
| | | HESLAQYNAKLDTRLLYPVSKYQQDQIVSR |
| PTN11_C^{sFes} | 158 | MKIEEHHHHHHSSGKLGLNDIFEAQKIEWHESSGED |
| | | LYFQSGTTMDDYKDDDDKGSLNCADPTSERWFHGH |
| | | LSGKEAEKLLTEKGKHGSFLVREGQSQPDFVLSVRT |
| | | GDDKGESNDGKSKVTHVIIRCQELKYDVGGGERFDS |
| | | LTDLVEHYKKNPMVETLGTVLQLKQPLNTTRINAAD |
| | | SR |
| PTN11_C^{sSrc} | 159 | MKIEEHHHHHHSSGKLGLNDIFEAQKIEWHESSGED |
| | | LYFQSGTTMDDYKDDDDKGSLNCADPTSERWFHGH |
| | | LSGKEAEKLLTEKGKHGSFLVRESETVKGAFALSVR |
| | | TGDDKGESNDGKSKVTHVLIRCQELKYDVGGGERF |
| | | DSLTDLVEHYKKNPMVETLGTVLQLKQPLNTTRINA |
| | | ADSR |
| PTN11_N^{sFes} | 160 | MKIEEHHHHHHSSGKLGLNDIFEAQKIEWHESSGED |
| | | LYFQSGTTMDDYKDDDDKGGSGSMTSRRWFHPNIT |
| | | GVEAENLLLTRGVDGSFLARPGQSQPDFVLSVRRNG |
| | | AVTHIIIQNTGDYYDLYGGEKFATLAELVQYYMEHH |
| | | GQLKEKNGDVIELKYPLNCADSR |
| PTN11_N^{sSrc} | 161 | MKIEEHHHHHHSSGKLGLNDIFEAQKIEWHESSGED |
| | | LYFQSGTTMDDYKDDDDKGGSGSMTSRRWFHPNIT |
| | | GVEAENLLLTRGVDGSFLARPSETVKGAFALSVRRN |
| | | GAVTHILIQNTGDYYDLYGGEKFATLAELVQYYME |
| | | HHGQLKEKNGDVIELKYPLNCADSR |
| PTN6_C^{sFes} | 162 | MKIEEHHHHHHSSGKLGLNDIFEAQKIEWHESSGED |
| | | LYFQSGTTMDDYKDDDDKGSLNCSDPTSERWYHGH |
| | | MSGGQAETLLQAKGEPWTFLVREGQSQPDFVLSVLS |
| | | DQPKAGPGSPLRVTHIIVMCEGGRYTVGGLETFDSLT |
| | | DLVEHFKKTGIEEASGAFVYLRQPYYATRVNAADSR |
| PTN6_C^{sSrc} | 163 | MKIEEHHHHHHSSGKLGLNDIFEAQKIEWHESSGED |
| | | LYFQSGTTMDDYKDDDDKGSLNCSDPTSERWYHGH |
| | | MSGGQAETLLQAKGEPWTFLVRESETVKGAFALSVL |
| | | SDQPKAGPGSPLRVTHILVMCEGGRYTVGGLETFDS |
| | | LTDLVEHFKKTGIEEASGAFVYLRQPYYATRVNAAD |
| | | SR |
| SH2D1B^{sSrc} | 164 | MKIEEHHHHHHSSGKLGLNDIFEAQKIEWHESSGED |
| | | LYFQSGTTMDDYKDDDDKGGSDLPYYHGRLTKQDC |
| | | ETLLLKEGVDGNFLLRDSETVKGALALCVSFKNIVY |
| | | TYLIFREKHGYYRIQTAEGSPKQVFPSLKELISKFEKP |
| | | NQGMVVHLLKPIKRTSSR |
| SHC_1^{sFes} | 165 | MKIEEHHHHHHSSGKLGLNDIFEAQKIEWHESSGED |
| | | LYFQSGTTMDDYKDDDDKGSSMAEQLRGEPWFHG |
| | | KLSRREAEALLQLNGDFLVREGQSQPDYVLTGLQSG |
| | | QPKHLILVDPEGVVRTKDHRFESVSHLISYHMDNHL |
| | | PIISAGSELCLQQPVERKLSR |
| SHC1^{sSrc} | 166 | MKIEEHHHHHHSSGKLGLNDIFEAQKIEWHESSGED |
| | | LYFQSGTTMDDYKDDDDKGSSMAEQLRGEPWFHG |
| | | KLSRREAEALLQLNGDFLVRESETVKGAYALTGLQS |
| | | GQPKHLLLVDPEGVVRTKDHRFESVSHLISYHMDNH |
| | | LPIISAGSELCLQQPVERKLSR |
| Srcs^{Fes} | 167 | MKIEEHHHHHHSSGKLGLNDIFEAQKIEWHESSGED |
| | | LYFQSGTTMDDYKDDDDKGGSDGIQAEEWYFGKIT |
| | | RRESERLLLNAENPRGTFLVREGQSQPDYVLSVSDFD |
| | | NAKGLNVKHYIIRKLDSGGFYITSRTQFNSLQQLVAY |
| | | YSKHADGLCHRLTTVCPSR |
| VAV^{sFes} | 168 | MKIEEHHHHHHSSGKLGLNDIFEAQKIEWHESSGED |
| | | LYFQSGTTMDDYKDDDDKGSGPPQDLSVHLWYAGP |
| | | MERAGAESILANRSDGTFLVRQGQSQPDFVISIKYNV |
| | | EVKHIIIMTAEGLYRITEKKAFRGLTELVEFYQQNSL |
| | | KDCFKSLDTTLQFPFKEPEKRTISSR |
| VAV^{sSrc} | 169 | MKIEEHHHHHHSSGKLGLNDIFEAQKIEWHESSGED |
| | | LYFQSGTTMDDYKDDDDKGSGPPQDLSVHLWYAGP |
| | | MERAGAESILANRSDGTFLVRQSETVKGAFAISIKYN |
| | | VEVKHILIMTAEGLYRITEKKAFRGLTELVEFYQQNS |
| | | LKDCFKSLDTTLQFPFKEPEKRTISSR |
| VAV2^{sFes} | 170 | MKIEEHHHHHHSSGKLGLNDIFEAQKIEWHESSGED |
| | | LYFQSGTTMDDYKDDDDKGSSREIDYTAYPWFAGN |
| | | MERQQTDNLLKSHASGTYLIREGQSQPDFVISIKEND |
| | | EVKHIIVVEKDNWIHITEAKKFDSLLELVEYYQCHSL |
| | | KESFKQLDTTLKYPYKSRERSASRSR |
| VAV2^{sSrc} | 171 | MKIEEHHHHHHSSGKLGLNDIFEAQKIEWHESSGED |
| | | LYFQSGTTMDDYKDDDDKGSSREIDYTAYPWFAGN |
| | | MERQQTDNLLKSHASGTYLIRESETVKGAFAISIKFN |
| | | DEVKHILVVEKDNWIHITEAKKFDSLLELVEYYQCH |
| | | SLKESFKQLDTTLKYPYKSRERSASRSR |
| VAV3^{sFes} | 172 | MKIEEHHHHHHSSGKLGLNDIFEAQKIEWHESSGED |
| | | LYFQSGTTMDDYKDDDDKGSPKPVDYSCQPWYAG |
| | | AMERLQAETELINRVNSTYLVRHGQSQPDYVISIKYN |
| | | NEAKHIIILTRDGFFHIAENRKFKSLMELVEYYKHHS |
| | | LKEGFRTLDTTLQFPYKEPEHSAGQSR |
| VAV3^{sSrc} | 173 | MKIEEHHHHHHSSGKLGLNDIFEAQKIEWHESSGED |
| | | LYFQSGTTMDDYKDDDDKGSPKPVDYSCQPWYAG |
| | | AMERLQAETELINRVNSTYLVRHSETVKGAYAISIKY |
| | | NNEAKHILILTRDGFFHIAENRKFKSLMELVEYYKHH |
| | | SLKEGFRTLDTTLQFPYKEPEHSAGQSR |
| PTN11_C^{sFes/αA-2} | 174 | MKIEEHHHHHHSSGKLGLNDIFEAQKIEWHESSGED |
| | | LYFQSGTTMDDYKDDDDKGSLNCADPTSERWFHGH |
| | | LSRKEAEKLLTEKGKHGSFLVREGQSQPDFVLSVRT |
| | | GDDKGESNDGKSKVTHVIIRCQELKYDVGGGERFDS |
| | | LTDLVEHYKKNPMVETLGTVLQLKQPLNTTRINAAD |
| | | SR |
| PTN11_C^{sSrc/αA-2} | 175 | MKIEEHHHHHHSSGKLGLNDIFEAQKIEWHESSGED |
| | | LYFQSGTTMDDYKDDDDKGSLNCADPTSERWFHGH |
| | | LSRKEAEKLLTEKGKHGSFLVRESETVKGAFALSVR |
| | | TGDDKGESNDGKSKVTHVLIRCQELKYDVGGGERF |
| | | DSLTDLVEHYKKNPMVETLGTVLQLKQPLNTTRINA |
| | | ADSR |
| PTN11_N^{sFes/αA-2} | 176 | MKIEEHHHHHHSSGKLGLNDIFEAQKIEWHESSGED |
| | | LYFQSGTTMDDYKDDDDKGGSGSMTSRRWFHPNIT |
| | | RVEAENLLLTRGVDGSFLARPGQSQPDFVLSVRRNG |
| | | AVTHIIIQNTGDYYDLYGGEKFATLAELVQYYMEHH |
| | | GQLKEKNGDVIELKYPLNCADSR |
| PTN11_N^{sSrc/αA-2} | 177 | MKIEEHHHHHHSSGKLGLNDIFEAQKIEWHESSGED |
| | | LYFQSGTTMDDYKDDDDKGGSGSMTSRRWFHPNIT |
| | | RVEAENLLLTRGVDGSFLARPSETVKGAFALSVRRN |
| | | GAVTHILIQNTGDYYDLYGGEKFATLAELVQYYME |
| | | HHGQLKEKNGDVIELKYPLNCADSR |
| PTN6_C^{sFes/αA-2} | 178 | MKIEEHHHHHHSSGKLGLNDIFEAQKIEWHESSGED |
| | | LYFQSGTTMDDYKDDDDKGSLNCSDPTSERWYHGH |
| | | MSRGQAETLLQAKGEPWTFLVREGQSQPDFVLSVLS |
| | | DQPKAGPGSPLRVTHIIVMCEGGRYTVGGLETFDSLT |
| | | DLVEHFKKTGIEEASGAFVYLRQPYYATRVNAADSR |
| PTN6_C^{sSrc/αA-2} | 179 | MKIEEHHHHHHSSGKLGLNDIFEAQKIEWHESSGED |
| | | LYFQSGTTMDDYKDDDDKGSLNCSDPTSERWYHGH |
| | | MSRGQAETLLQAKGEPWTFLVRESETVKGAFALSVL |
| | | SDQPKAGPGSPLRVTHILVMCEGGRYTVGGLETFDS |
| | | LTDLVEHFKKTGIEEASGAFVYLRQPYYATRVNAAD |
| | | SR |
| SH2D1B^{sSrc/αA-2} | 180 | MKIEEHHHHHHSSGKLGLNDIFEAQKIEWHESSGED |
| | | LYFQSGTTMDDYKDDDDKGGSDLPYYHGRLTRQDC |
| | | ETLLLKEGVDGNFLLRDSETVKGALALCVSFKNIVY |
| | | TYLIFREKHGYYRIQTAEGSPKQVFPSLKELISKFEKP |
| | | NQGMVVHLLKPIKRTSSR |

Provided here are pharmaceutical compositions comprising the modified SH2 domain described herein. In some aspects, the pharmaceutical compositions further comprise a pharmaceutically acceptable carrier, vehicle or diluent. In some aspects, the pharmaceutical compositions are administered to a subject in a biologically compatible form for administration in vivo. In some aspects, the pharmaceutical compositions are in the form of tablets, capsules, granules or powders. In some aspects, the pharmaceutical compositions are in the form of liquid or gel.

Also provided here are pharmaceutical compositions comprising a DNA expression vector comprising nucleic acids encoding the modified SH2 domain. In some aspects, the pharmaceutical compositions further comprise a pharmaceutically acceptable carrier, vehicle or diluent. In some aspects, the pharmaceutical compositions are administered to subjects in a biologically compatible form for administration in vivo. In some aspects, the pharmaceutical compositions are in the form of tablets, capsules, granules or powders. In some aspects, the pharmaceutical compositions are in the form of liquid or gel.

By “biologically compatible form suitable for administration in vivo” is meant a form of the substance to be administered in which any toxic effects are outweighed by the therapeutic effects. Administration of a therapeutically active amount of the pharmaceutical compositions described herein, or an “effective amount”, is defined as an amount effective at dosages and for periods of time, necessary to achieve the desired result of eliciting an immune response in a human. A therapeutically effective amount of a substance may vary according to factors such as the disease state/health, age, sex, and weight of the recipient, and the inherent ability of the particular polypeptide, nucleic acid coding therefore, or recombinant virus to elicit a desired immune response. In some aspects, dosage regiments are adjusted to provide the optimum therapeutic response. In some aspects, several divided doses are administered daily or on at periodic intervals, and/or the dose is proportionally reduced as indicated by the exigencies of the therapeutic situation. In some aspects, the amount of SH2 peptide for administration depends on the route of administration, time of administration and varied in accordance with individual subject responses.

In some aspects, the pharmaceutical composition described herein is administered by any suitable means. In some aspects, the pharmaceutical composition described herein is administered orally, sublingually, buccally, parenterally (such as by subcutaneous, intravenous, intramuscular, intraperitoneal or intrasternal injection or infusion techniques (e. g., as sterile injectable aqueous or non-aqueous solutions or suspensions)), nasally (such as by inhalation spray; topically, such as in the form of a cream or ointment), or rectally (such as in the form of suppositories). In some aspects, the pharmaceutical composition described herein is administered in dosage unit formulations containing non-toxic, pharmaceutically acceptable vehicles or diluents. In some aspects, the pharmaceutical composition described herein is administered in a form suitable for immediate release or extended release. Immediate release or extended release can be achieved using suitable pharmaceutical compositions comprising the present compounds, or, particularly in the case of extended release, by the use of devices such as subcutaneous implants or osmotic pumps. In some aspects, the pharmaceutical composition described herein is administered using liposomes.

The pharmaceutical compositions described herein can be prepared by methods for the preparation of pharmaceutically acceptable compositions which can be administered to subjects, such that an effective quantity of the active substance (i.e., SH2 variant peptide) is combined in a mixture with a pharmaceutically acceptable vehicle. In some aspects, the pharmaceutical compositions include, albeit not exclusively, solutions of the substances in association with one or more pharmaceutically acceptable vehicles or diluents and may be contained in buffered solutions with a suitable pH and/or be iso-osmotic with physiological fluids.

Pharmaceutical acceptable carriers are well known to those skilled in the art. In some aspects, the pharmaceutical acceptable carriers include sterile saline, lactose, sucrose, calcium phosphate, gelatin, dextrin, agar, pectin, peanut oil, olive oil, sesame oil, water, or any combination thereof. In some aspects, the pharmaceutical acceptable carriers are MHC class II molecules.

In some aspects, the pharmaceutical compositions described herein comprise one or more stabilizers. In some aspects, the stabilizers are carbohydrates including sorbitol, mannitol, starch, sucrose, dextrin and glucose, proteins such as albumin or casein, and buffers such as alkaline phosphates.

In some aspects, the pharmaceutical compositions described here comprise another therapeutic agents. In some aspects, the pharmaceutical compositions described herein are formulated by employing conventional solid or liquid vehicles or diluents. In some aspects, the pharmaceutical composition described herein is formulated with pharmaceutical additives of a type appropriate to the mode of desired administration. In some aspects, the pharmaceutical additives are excipients, binders, preservatives, stabilizers, flavors, or any combination thereof. In some aspects, the pharmaceutical composition is formulated according to techniques such as those well known in the art of pharmaceutical formulations.

In some aspects, The pharmaceutical compositions described herein comprises one or more additional suitable therapeutic agents useful in the treatment of protein tyrosine kinase-associated disorders. In some aspects, the additional therapeutic agents are PTK inhibitors, anti-inflammatories, anti-proliferatives, chemotherapeutic agents, immunosuppressants, anti-cancer agents, cytotoxic agents, or any combination thereof.

In some aspects, the pharmaceutical compositions described herein are employed alone or in combination with additional suitable therapeutic agents useful in cellular regeneration therapies. Cellular regeneration therapies include any treatment that requires stem cell activation or differentiation to re-plenish, grow, or re-seed tissues that are deficient in required cell types. Tissues of interest include, but are not limited to, skin, spinal cord, retinal, kidney or other tissues that can be treated in a similar fashion.

In some aspects, the additional therapeutic agents are cyclosporins (e. g., cyclosporin A), CTLA4-Ig, antibodies such as anti-ICAM-3, anti-IL-2 receptor (Anti-Tac), anti-CD45RB, anti-CD2, anti-CD3 (OKT-3), anti-CD4, anti-CD80, anti-CD86, monoclonal antibody OKT3, agents blocking the interaction between CD40 and gp39, such as antibodies specific for CD40 and/or gp39 (i.e., CD154), fusion proteins constructed from CD40 and gp39 (CD40Ig and CD8gp39), inhibitors, such as nuclear translocation inhibitors, of NF-kappa B function, such as deoxyspergualin (DSG), non-steroidal anti-inflammatory drugs (NSAIDs) such as ibuprofen, steroids such as prednisone or dexamethasone, gold compounds, anti-proliferative agents such as methotrexate, FK506 (tacrolimus, Prograf), mycophenolate mofetil, cytotoxic drugs such as azathioprine and cyclophosphamide, TNF-oc inhibitors such as tenidap, anti-TNF antibodies or soluble TNF receptor such as etanercept (Enbrel), rapamycin (sirolimus or Rapamune), leflunimide (Arava), and cyclooxygenase-2 (COX-2) inhibitors such as celecoxib (Celebrex) and rofecoxib (Vioxx), or derivatives thereof, the PTK inhibitors, or any combination thereof.

### Conditions for Treatment

Variant compositions comprising the modified SH2 domain of the present disclosure can be used to inhibit protein tyrosine kinases or phosphatases, and are thus useful in the treatment, including prevention and therapy, of protein tyrosine kinase/phosphatase-associated disorders such as immunologic and oncologic disorders.

The superSrc-SH2 (sSrc) and superFyn-SH2 (sFyn) are high affinity variants of the SH2 domains of the Src and Fyn tyrosine kinases, respectively. Expression of autonomous SuperSrc-SH2 and superFyn-SH2 was shown to exert a dominant negative effect that antagonized cancer cell signaling. In contrast, expression of a high affinity variant of the Grb2 SH2 domain (sGrb2), in the context of the whole protein, exerted a dominant positive effect that stimulated stem cell differentiation pathways. It follows, that variants of sSrc and sFyn developed according to the present invention also exert a dominant negative effect that antagonizes cancer cell signaling, and that variant of the sGrb2 created according to the present invention exerts a dominant positive effect that stimulates stem cell differentiation pathways.

The compounds also inhibit receptor tyrosine kinases including EGFR and are therefore useful in the treatment of proliferative disorders such as psoriasis and cancer. The ability of these variant SH2 to inhibit EGFR and other receptor kinases will also permit their use as anti-angiogenic agents to treat disorders such as cancer and diabetic retinopathy. “Protein tyrosine kinase-associated disorders” are those disorders which result from aberrant tyrosine kinase activity, and/or which are alleviated by the inhibition of one or more of these enzymes. For example, Lck inhibitors are of value in the treatment of several such disorders (for example, the treatment of autoimmune diseases), as Lck inhibition blocks T cell activation. The treatment of T cell mediated diseases, including inhibition of T cell activation and proliferation, is one implementation of the methods and compositions described herein. Compounds which selectively block T cell activation and proliferation may be desirable. Compounds provided herein which block the activation of endothelial cell PTK by oxidative stress, thereby limiting surface expression of adhesion molecules that induce neutrophil binding, and which inhibit PTK necessary for neutrophil activation are useful, for example, in the treatment of ischemia and reperfusion injury.

Provided here are example methods for the treatment of protein tyrosine kinase/phosphatase-associated disorders, comprising administering to a subject in need thereof the pharmaceutical composition described herein in an amount effective. In some aspects, the methods further comprise administering to a subject in need thereof additional therapeutic agents such as those described below may be employed with the inventive compounds in the present methods. In some aspects, the additional therapeutic agents may be administered prior to, simultaneously with or following the administration of the compositions of the present disclosure. In some aspects, the compositions may be provided as a fused product to a membrane penetrating peptide such as a TAT protein transduction domain. The compositions may also be provided within a carrier that allows transportation across a cell membrane.

In some aspects, the disorders include, but are not limited to, transplant (such as organ transplant, acute transplant or heterograft or homograft (such as is employed in burn treatment)) rejection; protection from ischemic or reperfusion injury such as ischemic or reperfusion injury incurred during organ transplantation, myocardial infarction, stroke or other causes; transplantation tolerance induction; arthritis (such as rheumatoid arthritis, psoriatic arthritis or osteoarthritis); multiple sclerosis; chronic obstructive pulmonary disease (COPD), such as emphysema; inflammatory bowel disease, including ulcerative colitis and Crohn's disease; lupus (systemic lupus erythematosus); graft versus host disease; T-cell mediated hypersensitivity diseases, including contact hypersensitivity, delayed-type hypersensitivity, and gluten-sensitive enteropathy (Celiac disease); psoriasis; contact dermatitis (including that due to poison ivy); Hashimoto's thyroiditis; Sjogren's syndrome; Autoimmune Hyperthyroidism, such as Graves' Disease; Addison's disease (autoimmune disease of the adrenal glands); autoimmune polyglandular disease (also known as autoimmune polyglandular syndrome); autoimmune alopecia; pernicious anemia; vitiligo; autoimmune hypopituitarism; Guillain-Barre syndrome; other autoimmune diseases; cancers, including cancers where Lck or other Src-family kinases such as Src are activated or overexpressed, such as colon carcinoma and thymoma, and cancers where Src-family kinase activity facilitates tumor growth or survival; glomerulonephritis; serum sickness; urticaria; allergic diseases such as respiratory allergies (asthma, hay fever, allergic rhinitis) or skin allergies; scleroderma; mycosis fungoides; acute inflammatory responses (such as acute respiratory distress syndrome and ischemia/reperfusion injury); dermatomyositis; alopecia areata; chronic actinic dermatitis; eczema; Bechet's disease; Palmoplantar Pustulosis; Pyoderma gangrenosum; Sezary's syndrome; atopic dermatitis; systemic sclerosis; and morphea. In some aspects, the method further comprises The present disclosure also provides a method for treating the aforementioned disorders by administering any compound capable of inhibiting protein tyrosine kinase.

Src-family kinases other than Lck, such as Hck and Fgr, are important in the Fc gamma receptor responses of monocytes and macrophages. Compounds of the present disclosure inhibit the Fc gamma dependent production of TNF alpha in the monocyte cell line THP-1 that does not express Lck. The ability to inhibit Fc gamma receptor dependent monocyte and macrophage responses results in additional anti-inflammatory activity for the present compounds beyond their effects on T cells. This activity is especially of value, in some aspects, in the treatment of inflammatory diseases such as arthritis or inflammatory bowel disease.

In particular, the present SH2 superbinder domains are of value for the treatment of autoimmune glomerulonephritis and other instances of glomerulonephritis induced by deposition of immune complexes in the kidney that trigger Fc gamma receptor responses leading to kidney damage.

In addition, Src family kinases other than Lck, such as Lyn and Src, are important in the Fc epsilon receptor induced degranulation of mast cells and basophils that plays an important role in asthma, allergic rhinitis, and other allergic disease. Fc epsilon receptors are stimulated by IgE-antigen complexes. Variant SH2s of the present disclosure inhibit the Fc epsilon induced degranulation responses, including in the basophil cell line RBL that does not express Lck. The ability to inhibit Fc epsilon receptor dependent mast cell and basophil responses results in additional anti-inflammatory activity for the present compounds beyond their effect on T cells. In particular, the present compounds are of value for the treatment of asthma, allergic rhinitis, and other instances of allergic disease.

The combined activity of the present variant SH2 towards monocytes, macrophages, T cells, etc. may be of value in the treatment of any of the aforementioned disorders. Additionally, Zap70 is involved in initiating and controlling the intensity and duration of T cell receptor signaling. Variant SH2s of the present disclosure, including but not limited to Zap70 or SH2s variant combinations, can be used to modulate abnormal immune responses and develop synthetic switches for designing chimeric antigen receptor (CAR) T cells with desired performances.

In some aspects, the modified SH2 domains disclosed herein are used for inducing stem cell differentiation for the treatment of diseases such as, but not limited to, epidermolysis bullosa, myocardial infarction, disorders associated with loss of vision, Duchenne's muscular dystrophy. In some aspects, the modified SH2 domains trigger stem cell fate change and lineage specification.

Also provided here are methods of treating proliferative diseases comprising administering to a subject the pharmaceutical composition provided herein. In some aspects, the proliferative diseases include psoriasis and cancer. In some aspects, the cancer is non-small cell lung cancer, colorectal cancer, or breast cancer. Similarly, the HER2 receptor kinase has been shown to be overexpressed in breast, ovarian, lung and gastric cancer. Monoclonal antibodies that downregulate the abundance of the HER2 receptor or inhibit signaling by the HER1 receptor have shown anti-tumor efficacy in pre-clinical and clinical studies. It is therefore expected that inhibitors of the HER1 and HER2 kinases will have efficacy in the treatment of tumors that depend on signaling from either of the two receptors. These compounds are expected to have efficacy either as single agent or in combination with other chemotherapeutic agents such as placlitaxel (Taxol), doxorubicin hydrochloride (adriamycin), and cisplatin (Platinol). The above other therapeutic agents, which are not exhaustive, when employed in combination with the compounds of the present disclosure, may be used as determined by one of ordinary skill in the art.

### Diagnosis

Described herein are methods of diagnosing protein tyrosine kinase-associated disorders in a subject comprising (a) contacting a sample taken from the subject with at least one immobilized peptide comprising the modified SH2 domain; (b) isolating polypeptides bound by the at least one immobilized peptide, wherein the polypeptides are pTyr-containing polypeptides; (c) measuring the level of the pTyr-containing polypeptides in the sample using a spectroscopic, photochemical, biochemical, immunochemical, or chemical technique; (d) comparing the level of the pTyr-containing polypeptides in (c) with a control. In some aspects, the modified SH2 domain is an sSH2 domain described herein. In some aspects, the disorder is cancer.

Also provided herein are methods of screening cancer cells comprising: (a) lysing a sample to obtain cell lysates, wherein the sample comprises cells; (b) contacting the cell lysates and a control with a composition comprising one or more of the modified SH2 domains described herein; (c) collecting proteins with phosphorylated tyrosine bound to the modified SH2 domain; (d) comparing a level of the proteins with phosphorylated tyrosine in the sample to a level of the proteins with phosphorylated tyrosine in the control.

For the methods disclosed herein, the term “sample” or “biological sample” refers to cells, tissues, organs, bacteria, bodily fluids and so forth obtained from a subject. Bodily fluids include blood (whole blood, blood plasma, blood serum, capillary blood, venous blood), tears, spinal fluid, synovial fluid, bronchoalveolar fluid, bronchoalveolar lavage, tissue extracts, urine, sweat, saliva, excrement, phlegm or other like bodily fluids excreted from a subject.

The samples used in any of the methods of the present disclosure may be analyzed using any suitable quantifiable or semi-quantifiable spectroscopic, photochemical, biochemical, immunochemical, or chemical means technique, including (1) mass spectrometry (MS), tandem mass spectrometry (MS-MS), and/or MS3 analysis, SPECT, CT and PET imaging, enzyme linked immunosorbent assay (ELISA), luciferase or any other technique or assay similar to ELISA/luciferase assay especially involving a microtiter plate.

Provided herein are methods for detecting a protein tyrosine kinase in a biological sample. In some aspects, the methods include: contacting a sample with one or more peptides comprising modified SH2 domain described herein, wherein the one or more modified SH2 domain or peptide comprising the modified SH2 domain having a detectable label; applying an imaging technique for detecting the label in the sample, wherein detection of the label in the sample indicates the presence of the protein kinase or pTyr containing peptide in the sample.

Provided herein are methods for diagnosing a pTyr associated disorder in a subject, said method comprising: contacting a sample taken from said subject with one or more of the modified SH2 domain described herein or with one or more peptides comprising the modified SH2 domain described herein, the modified SH2 domain comprising the SH2 variant having a detectable label; and applying an imaging technique for detecting the label in the sample. In some aspects, the sample is a bodily fluid, cell, or tissue. Detection of the SH2 variant/protein tyrosine kinase complex or pTyr containing polypeptide in the sample being indicative of a positive diagnosis of the disorder or condition. In aspects such disorder may be, but not limited to, cancer.

Provided herein are methods for diagnosing a disorder or condition associated with altered levels of pTyr in a subject includes: (a) contacting a sample taken from said subject with at least one immobilized peptide comprising the modified SH2 variant described herein, (b) isolating polypeptides bound by the at least one immobilized peptide, thereby isolating pTyr-containing polypeptides from the sample; (c) measuring the level of pTyr-containing polypeptides in the sample; (d) comparing the level measured in (c) with a normal control or baseline level; and (e) determining whether the subject has the disorder or condition based on the comparison of (d). In some aspects, the level of pTyr-containing polypeptides in the sample is measured mass spectrometry, ELISA, luciferase detection or any other method using microtiter plates

Provided herein are methods of prognosing a disorder or condition associated with altered levels of pTyr in a subject, the method comprising (a) contacting a sample taken from said subject with at least one immobilized peptide comprising the modified SH2 domain described herein, (b) isolating polypeptides bound by the at least one immobilized peptide, thereby isolating pTyr-containing polypeptides from the sample; (c) measuring the level of pTyr-containing polypeptides in the sample using a spectroscopic, photochemical, biochemical, immunochemical, or chemical technique such as mass spectrometry, ELISA, luciferase detection or any other method using microtiter plates; (d) comparing the level measured in (c) with a normal control (a negative control) or with a baseline level or previously analyzed sample pertaining to the same or similar disease or condition being analyzed; and (e) correlating potential outcomes for the subject with the disorder or condition based on the comparison of (d).

Further provided herein are methods of evaluating the efficiency of a treatment of a disorder or condition associated with altered levels of pTyr in a subject that is being treated for said disorder or condition, the method comprising: (a) contacting samples taken from said subject at different times during the treatment with at least one immobilized peptide comprising a SH2 variant of the present disclosure, (b) isolating polypeptides bound by the at least one immobilized peptide, thereby isolating pTyr-containing polypeptides from each of the samples at the different times; (c) measuring the level of pTyr-containing polypeptides in each of the samples at the different times using a spectroscopic, photochemical, biochemical, immunochemical, or chemical technique; (d) comparing the level measured in (c) with a normal control level (negative control) or with baseline level taken from the subject prior to starting the treatment or with a previously analyzed sample being treated with a drug, pharmaceutical intervention, diet regime or any medical intervention being used to treat the same or similar disease or condition thereby following the efficiency of the treatment. In some aspects, the level of pTyr-containing polypeptides is measured using mass spectrometry, ELISA, luciferase detection or any other method using microtiter plates. Returns to a normal level of the pTyr-containing polypeptides in a subject undergoing treatment for a disorder associated with altered levels of pTyr can serve as an aid in following medical interventions (including rehabilitation therapy) of said subjects. A return to a normal level of the pTyr-containing polypeptides in the subject relative to the negative control serves to assess whether the treatment (including rehabilitation therapy) of the subject has been successful.

In some aspects, a subject's sample may be contacted with the modified SH2 domain described herein using mass spectrometry, ELISA, luciferase detection or any other method using microtiter plates with a patient's tissue extracts or liquid samples such as, but not limited to, blood, urine saliva, sweat. An advantage compared to using anti-pTyr antibody is that the modified SH2 domain has detection specificity to particular cancer pTyr site sequences. An advantage compared to the wild-type SH2 domain is that the modified SH2 domain exhibit tighter binding.

Further provided herein are libraries comprising the compositions described herein. In some aspects, a library of measurements of pTyr is established for diagnosed cases of any disorders or conditions associated with altered levels of pTyr. In some aspects, a library of measurements of pTyr is established for different types of cancers. In some aspects, this library is used as the predetermined, control set of the pTyr measurements of cancer (referred to as positive control). In some aspects, a comparison may be made of the subject's pTyr measurements against the predetermined pTyr measurements of cancer and the predetermined pTyr measurements of non-cancer (referred to as negative control or normal control) to determine not only if the patient has cancer, but also the type of cancer and the prognosis.

In some aspects, the diagnosing comprises staining of diseased and normal tissues, with a disease-specific variant SH2 domain. In some aspects, the diagnosing comprises comparing the binding profiles of normal and disease cell lysates. In some aspects, the diagnosing comprises injecting a radiolabeled and maybe TAT-tagged variant SH2 domain to a cancer patient to detect SH2 accumulation to a tumor site in the patient's body. In some aspects, to detect the SH2 variant in the samples, the modified SH2 domains are labelled with a probe molecule.

In some aspects, the modified SH2 domains disclosed herein are used in methods of identifying pTyr-positive cells. In some aspects, the methods comprise using one or more of the variant SH2 to detect for the presence of pTyr-positive cells in a sample. In some aspects, pTyr-positive sample is indicative that the sample contains cancerous cells.

Further provided here are methods of prognosing cancer comprising detecting the presence or absence of pTyr-containing cells in a sample, whereby the presence of the pTyr-containing cells in the sample may indicates that an aggressively metastatic cancer cell is present in the sample.

In some aspects, the detection of pTyr-positive cells is carried out by a probe. In some aspects, the probe includes at least a peptide comprising a SH2 variant and an imaging component. In some aspects, this probe is labelled with a detectable marker which may allow detection of the location of the pTyr-positive cells. In some aspects, the probe may allow following movement and development of pTyr-positive cells. In some aspects, the imaging component of the probe comprises a label. Methods of labelling are well known to those of skill in the art. In some aspects, the label is suitable for use in in vivo imaging. In some aspects, the SH2 superbinder probes are labelled prior to detection. In some aspects, the label binds to the hybridization product. In some aspects, the labels are detectable. In some aspects, the labels that are detectable includes, without limitation, any material having a detectable physical or chemical property and have been well-developed in the field of immunoassays. In some aspects, the label is any composition detectable by spectroscopic, photochemical, biochemical, immunochemical, or chemical means.

Labels which may be used in the present disclosure include biotin-based label, magnetic label (e.g. DYNABEADS™, ThermoFisher Scientific, Waltham, MA MAGNE™ Streptavidin Beads, Promega, Madison, WI), paramagnetic labels, radioactive label (e.g. ³H, ³⁵S, ³²P, ⁵¹Cr, or ¹²⁵I), fluorescent label (e.g. fluorescein, rhodamine, Texas Red, etc.), fluorescent proteins (i.e. GFP, RFP, CFP) electron-dense reagents (e.g. gold), enzymes (e.g. alkaline phosphatase, horseradish peroxidase, luciferase or others commonly used in an ELISA), digoxigenin, or haptens and proteins for which antisera or monoclonal antibodies may be available (In some aspects, the peptides of the present disclosure can be made detectable by, In some aspects, incorporating a radiolabel into the peptide, and used to detect antibodies specifically reactive with the peptide). In some aspects, the modified SH2 domain described herein is provided with a carrier. In some aspects, the SH2 domain is coupled to bovine serum albumin (BSA) or keyhole limpet haemocyanin. In some aspects, the modified SH2 domain is covalently or non-covalently coupled to a solid carrier. In some aspects, the modified SH2 domain is coupled to a microsphere of gold or polystyrene, a slide, a chip, or to a wall of a microtiter plate. In some aspects, the modified SH2 domain is labelled directly or indirectly with a label selected from but not limited to biotin, fluorescein, and an enzyme such as horseradish peroxidase.

The particular label used may not be critical to the present disclosure, so long as it does not interfere with the affinity of the SH2 variant for the pTyr. In some aspects, the imaging component is a radionuclide (e.g., ¹⁸F, ¹¹C, ¹³N, ⁶⁴Cu, ⁶⁸Ga, ¹²³I, ¹¹¹In, ⁹⁹mTc, etc.) due to the ease of using such techniques as SPECT, CT and PET imaging for in vivo detection of SH2 variant-pTyr complexes and tumor cells. Decision as to appropriate imaging component for agents used in SPECT or PET imaging may also be determined by whether the radionuclide is generated by generator or cyclotron or is a chelator or organic/halide.

A direct labelled probe, as used herein, may be a probe to which a detectable label is attached. Because the direct label is already attached to the probe, no subsequent steps may be required to associate the probe with the detectable label. In contrast, an indirect labeled probe may be one which bears a moiety to which a detectable label is subsequently bound, typically after the SH2 variant peptide is bound with the target pTyr.

In some aspects, monoclonal antibodies (mAb) which recognize any of the modified SH2 domains of the disclosure are made and used to detect the presence of the variant SH2 in a sample. mAb's provide a rapid and simple method of testing the compositions of the disclosure for their quality. Any suitable methods for the preparation of antibodies may be employed. In some aspects, methods to produce mAb which specifically recognize the Variant SH2 of the disclosure are well known to those of skill in the art. In general, peptides are injected in Freund's adjuvant into mice or rabbit. After being injected 9 times over a three-week period, the mice spleens are removed and re-suspended in phosphate buffered saline (PBS). The spleen cells may serve as a source of lymphocytes, some of which may be producing antibody of the appropriate specificity. These may then be fused with a permanently growing myeloma partner cell, and the products of the fusion may be plated into several tissue culture wells in the presence of a selective agent such as HAT. The wells may then be screened to identify those containing cells making useful antibody by ELISA. These may then be freshly plated. After a period of growth, these wells may again be screened to identify antibody-producing cells. Several cloning procedures may be carried out until over 90% of the wells contain single clones which are positive for antibody production. From this procedure stable lines of clones may be established which produce the mAb. The mAb may then be purified by affinity chromatography using Protein A or Protein G Sepharose.

### Additional Applications

In some aspects, the modified SH2 domains, or a gene that encode one or more of the modified SH2 domains, may be introduced into a mammalian cell line.

In some aspects, the modified SH2 domains described herein that exhibit super-high affinity to a target pTyr site (K_{d} value smaller than about 100 nM) act to mask the target pTyr site and may cause severe blocking effects of PTK signaling events downstream of the pTyr site. Therefore, in some aspects, the modified SH2 domains described herein that exhibit super-high affinity to a target pTyr site serves as an inhibitory reagent of cellular PTK signaling pathway. Super-high affinity modified SH2 domains derived from different natural SH2 domains exhibit distinct sequence recognition specificity. Consequently, a super-high affinity modified SH2 domain, when introduced in a live cell, may block a specific signaling pathway, and may be used as a reagent for investigating physiology of a particular pathway.

In some aspects, the sSH2 variants described herein is used as substitutes for an anti-pTyr antibody and may be used in research areas where an anti-pTyr antibody is used, such as, in some aspects, Western blots, ELISA, luciferase assay, LUMIER assay, proteomics (enrichment of phosphoproteins/peptides), microscopy and so forth.

The modified SH2 domains of the present disclosure that exhibit moderately enhanced affinity (variants that show enhanced affinity compared to the wild type, and in one implementation with a K_{d} value greater than about 100 nM to a target pTyr site) may be produced in accordance with the present disclosure. These modified SH2 domains do not have an ability to completely block a pTyr site and its downstream signaling, but they may retain inherent sequence recognition specificity of a parent SH2 domain to which amino acid substitutions are applied. Therefore, these modified SH2 domains may be used as tracers or biosensors of particular tyrosine phosphorylation events in cells. To detect the tracer SH2 domain in cells, in some aspects, the SH2 domain may be labelled with a probe molecule, as explained above. These bio-sensors or tracers can be transiently or stably transfected/transformed or delivered using special protein tags as previously described into cells (i.e. mammalian cells, bacteria, single celled organisms, multi-celled organisms or other similar biological systems).

### Advantages

The modified SH2 domains of the present disclosure, used either alone or in combination with one another, is superior to conventional technologies with respect to some attributes. (1) High Affinity: the majority of the SH2 superbinders of the present disclosure have ultra-high affinity for pTyr (0.8-100 nM). This allows the SH2 superbinders of the present disclosure to probe pTyr signals with greater depth. (2) Specificity: since Applicants have engineered different SH2 domains with different specificity profiles, a more diverse range of pTyr peptides can now be recovered (other technologies are biased as to which pTyr peptides they bind to). This allows SH2 superbinders of the present disclosure to probe pTyr signals with greater coverage. (3) Cost: SH2 superbinders can be easily expressed in tens of milligrams in bacteria. This means one can cheaply produce thousands of samples using standard laboratory procedures for protein production. (4) Protein stability/developability: the present disclosure demonstrates that SH2 superbinders are still fully functional even years after being frozen and weeks of storage at 4° C. (5) Scalability: due to the low cost and molecular weight of SH2 superbinders, these proteins are more amenable to scaling for large numbers of samples in typical mass spectrometry workflows.

Numerous variations, changes, and substitutions will now occur to those skilled in the art without departing from this disclosure. It should be understood that various alternatives to the exemplifications of this disclosure described herein are employed in practicing the disclosure. It is intended that the following claims define the scope of the disclosure and that methods and structures within the scope of these claims and their equivalents be covered thereby.

### Additional Methods and Compositions

Provided herein is a method of producing a modified Src homology 2 (SH2) domain having equal or increased binding affinity to a pTyr-containing ligand relative to its unmodified version of the SH2 domain (SH2 domain to be modified), the SH2 domain to be modified domain having a pTyr binding pocket (αA2), a three-strand anti-parallel β-sheet including a B anti-parallel β-sheet (βB), a C anti-parallel β-sheet (βC) and a D anti-parallel β-sheet (BD), the three-strand anti-parallel β-sheet being flanked by a two α-helices, the βB being separated from βC by a BC loop, the method comprising: a) aligning the amino acid sequence of the SH2 domain to be modified using a suitable alignment algorithm to multiple SH2 domain sequences to determine the amino acid positions of αA2, the three-strand anti-parallel β-sheet and the two α-helices; and at least one or both of b1) replacing the BC loop (amino acid positions 59 to 68) of the SH2 domain to be modified with a BC loop motif of a superbinder SH2 domain known to exhibit increased binding affinity for the pTyr-containing ligand relative to a wild-type version of the SH2 domain superbinder, and b2) when the SH2 domain to be modified lacks an Arg residue at the αA2 position (position 33), substituting the amino acid at the position αA2 of the SH2 domain to be modified with an Arg residue, thereby producing the modified SH2 domain having equal or increased binding affinity to the pTyr-containing ligand. In some aspects, when the SH2 domain to be modified contains hydrophilic amino acid residues at amino acid position 70 (PC2) and at amino acid position 102 (βD6), then the method further comprises replacing each of said hydrophilic amino acid residues at position βC2 and at position βD6 with a hydrophobic amino acid residue, wherein the amino acid position 70 and the amino acid position 102 of the SH2 domain to be modified are determined from the sequence alignment of (a). In some aspects, the superbinder SH2 domain is a superbinder Src SH2 domain (sSrc, SEQ ID NO:136), and the BC loop motif has an amino acid sequence of SETVKGA (SEQ ID NO: 129). In some aspects, the method further comprises replacing the amino acid at position βC2 (position 70) and the amino acid at position βD6 (position 102) of the SH2 domain to be modified with an Ala residue and a Leu residue respectively, wherein the amino acid position 70 and the amino acid position 102 of the SH2 domain to be modified are determined from the sequence alignment of (a). In some aspects, the superbinder SH2 domain is a superbinder Fes SH2 domain variant (sFes1) having an amino acid sequence of SEQ ID NO: 130, and the BC loop motif has an amino acid sequence of GQSQPD (SEQ ID NO: 123). In some aspects, the superbinder SH2 domain is a superbinder Fes SH2 domain variant (sFes2) having an amino acid sequence of SEQ ID NO: 131, and the BC loop motif has an amino acid sequence of RQRKQE (SEQ ID NO: 124). In some aspects, the superbinder SH2 domain is a superbinder Fes SH2 domain variant having an amino acid sequence of SEQ ID NO: 132 (sFes3), and the BC loop motif has an amino acid sequence of SPRIQE (SEQ ID NO: 125). In some aspects, the superbinder SH2 domain is a superbinder Fes SH2 domain variant having an amino acid sequence of SEQ ID NO: 133 (sFes4), and the BC loop motif has an amino acid sequence of SQGRKV (SEQ ID NO: 126). In some aspects, the superbinder SH2 domain is a superbinder Fes SH2 domain variant having an amino acid sequence of SEQ ID NO: 134 (sFes5), and the BC loop motif has an amino acid sequence of SISKQG (SEQ ID NO: 127). In some aspects, the superbinder SH2 domain is a superbinder Fes SH2 domain variant having an amino acid sequence of SEQ ID NO: 135 (sFes6), and the BC loop motif has an amino acid sequence of SQTYPG (SEQ ID NO: 128). In some aspects, the method further comprises replacing the amino acid at position βC2 (position 70) and the amino acid at position βD6 (position 102) of the SH2 domain to be modified with a Val residue and an Ile residue respectively, wherein the amino acid position 70 and the amino acid position 102 of the SH2 domain to be modified are determined from the sequence alignment of (a). In some aspects, the multiple SH2 domain sequences include the SH2 domain sequences found in Table 1 (SEQ ID NOS: 1-122).

Provided herein is a Src homology 2 (sSH2) domain having an amino acid sequence selected from SEQ ID NOS: 130 to 180.

Provided herein is a peptide comprising an SH2 domain of the present disclosure. In some aspects, the peptide includes or is conjugated to, a detectable label.

Provided herein is a BC loop of a Src homology 2 (SH2) domain having an amino acid sequence selected from SEQ ID NOS: 123-129.

Provided herein is an affinity isolation device for isolating pTyr-containing polypeptides from a sample, the affinity isolation device comprising one or more immobilized peptides comprising the SH2 domains of the present disclosure. In some aspects, the one or more immobilized peptides are immobilized to streptavidin beads and the peptides are biotinylated.

Provided herein is a method for isolating a pTyr-containing polypeptide from a complex mixture of peptides, the method comprising: (a) contacting a proteinaceous preparation with at least one immobilized peptide comprising one or more of the peptides comprising an SH2 domain of the present disclosure; and (b) isolating polypeptides bound by the at least one immobilized peptide comprising the one or more peptides comprising an SH2 domain of the present disclosure in step (a), thereby isolating the pTyr-containing polypeptide. In some aspects, the method further comprises (c) characterizing the polypeptides isolated in step (b) by mass spectrometry (MS), tandem mass spectrometry (MS-MS), and/or MS3 analysis. In some aspects, the method further comprises (d) utilizing a search program to substantially match the spectra obtained for the polypeptides isolated in step (b) during the characterization of step (c) with the spectra for a known peptide sequence, thereby identifying parent proteins of the isolated polypeptide.

Provided herein is a method for detecting a protein tyrosine kinase in a biological sample comprising: (a) contacting the biological sample with the peptide comprising an SH2 domain of the present disclosure; and (b) applying an assay for detecting the label in the biological sample, wherein detection of the label in the biological sample indicates the presence of the protein kinase in the biological sample.

Provided herein is a method of diagnosing a subject of a disorder or condition associated with altered levels of pTyr comprising: (a) obtaining a bodily fluid, cell or tissue sample from the subject; (b) contacting the sample with the peptide comprising an SH2 domain of the present disclosure; and (c) applying an assay for detecting the label in the sample, wherein detection of the label in the sample indicates a positive diagnosis of the disorder or condition. In some aspects, the method further comprises (d) comparing a signal of the label detected in (c) with the signal obtained from contacting the peptide provided herein to a normal control sample, wherein a difference in the signal from the sample and the signal in the normal control indicates a positive diagnosis of the disorder or condition. In some aspects, the assay for detecting the label includes spectroscopic, photochemical, biochemical, immunochemical, or chemical techniques.

Provided herein is a method for diagnosing a disorder or condition associated with altered levels of pTyr in a subject, said method comprising: (a) contacting a sample taken from said subject with at least one immobilized peptide comprising one or more of the peptides comprising an SH2 domain of the present disclosure, (b) isolating polypeptides bound by the at least one immobilized peptide, thereby isolating pTyr-containing polypeptides from the sample; (c) measuring the level of pTyr-containing polypeptides in the sample using a spectroscopic, photochemical, biochemical, immunochemical, or chemical technique; (d) comparing the level measured in (c) with a normal control level; and (e) determining whether the subject has the disorder or condition based on the comparison of (d). In some aspects, only when the subject is diagnosed with the disorder or condition, the method further comprises treating the subject for said disorder or condition.

Provided herein is a method for providing a prognosis of a disorder or condition associated with altered levels of pTyr in a subject, the method comprising: (a) contacting a sample taken from said subject with at least one immobilized peptide comprising a SH2 variant of the present disclosure, (b) isolating polypeptides bound by the at least one immobilized peptide, thereby isolating pTyr-containing polypeptides from the sample; (c) measuring the level of pTyr-containing polypeptides in the sample using a spectroscopic, photochemical, biochemical, immunochemical, or chemical technique; (d) comparing the level measured in (c) with a normal control level or baseline level taken from the subject; and (e) correlating potential outcomes for the subject with the disorder or condition based on the comparison of (d).

Provided herein is a method for following the efficiency of a treatment of a disorder or condition associated with altered levels of pTyr in a subject, the method comprising: (a) contacting samples taken from said subject at different times during the treatment with at least one immobilized peptide comprising a SH2 variant of the present disclosure, (b) isolating polypeptides bound by the at least one immobilized peptide, thereby isolating pTyr-containing polypeptides from each of the samples at the different times; (c) measuring the level of pTyr-containing polypeptides in each of the samples at the different times using a spectroscopic, photochemical, biochemical, immunochemical, or chemical technique; and (d) comparing the level measured in (c) with a normal control level or baseline level taken from the subject prior to commencement of the treatment to follow the efficiency of the treatment.

### EXAMPLES

The examples below further illustrate the described aspects without limiting the scope of this disclosure.

### Example 1—Methods

### Sequence and Structural Alignments

The amino acid sequences of all human SH2 and SH2-like domains were downloaded from UniProt, and their amino acid sequences were aligned using COBALT from NCBI (Table 1).

For structural alignment of SH2 domains, all available structures were retrieved from the Protein Data Bank (PDB). Custom scripts were used for importing structures and performing the alignments in PyMol.

### Construction of Fes SH2 Library and Superbinder Selection

The Fes SH2 library was generated using combinatorial site-directed mutagenesis of a phagemid vector designed for the display on the major coat protein P3 of the Fes SH2 domain (residues 448-550). A total of 13 residues, encompassing three regions were mutated with a soft randomization strategy as previously described. Mutagenic oligonucleotides were synthesized to diversify adjusting the nucleotide ratio of diversified positions to 70% of the wild-type nucleotide and 10% of each of the other nucleotides. The obtained library had a diversity of 1.6×10¹⁰.

Selections of Fes superbinders were performed as previously described with minor modifications. 50 ng/well of streptavidin and neutravidin in PBS pH 7.4 were used on alternating days to immobilize on MAXISORP plates (Thermo Fisher Scientific, Waltham, MA) the biotinylated Ezrin phosphopeptide (PV{pTyr}PPVS (SEQ ID NO: 254)). The concentration of plate-immobilized phosphopeptide was decreased after each round of selection from 100 nM to 10 nM. To avoid binding to the non-phosphorylated peptide, the phage-displayed library was depleted on 100 nM of biotinylated Ezrin peptide lacking phosphorylation.

After 5 rounds of selections, 96 clones were picked and assayed for binding by phage ELISAs to identify clones able to bind to the pTyr-peptide but not to the non-phosphorylated peptide. Amino acid sequences of selected Fes superbinders were determined by Sanger DNA sequencing (Table 1; SEQ IDs: 130-135).

### Peptide Synthesis

Peptide were synthesized using 9-fluorenylmethoxycarbonyl chemistry with the Prelude peptide synthesizer (Gyros Protein Technologies, Inc., Tucson, AZ) on Rink amide MBHA resin (Novabiochem, London, GB). Each peptide N-terminus was functionalized with biotin through a linker composed of two F-aminocaproic acids (Bachem). N-hydroxysuccinimide-fluorescein was used for N-terminal fluorescein labeling of the synthesized Ezrin pTyr-peptide used in fluorescence polarization tests. All peptides were purified using C-18 reverse phase HPLC (Waters) and their identity was confirmed by mass spectrometry on an Orbitrap Elite (ThermoFisher). Phosphopeptides that were used in the study but not synthesized in house were from GenScript.

### Fluorescence Polarization and Peptide Synthesis

Fluorescence polarization assays were performed as previously described. Briefly, binding measurements were performed in FP buffer (25 mM HEPES pH 7.5, 100 mM NaCl, 1 mM DTT, 0.01 mg/ml BSA, 0.03% BRIJ-35) by mixing in a 96-well plate 25 nM FITC-labeled Ezrin pTyr-peptide with serial dilutions of Fes-SH2 variants ranging from 1 mM to 22 nM. Samples were equilibrated at room temperature for 30 minutes before reading plates on an HTS Multi-Mode Microplate Reader (Synergy Neo) using an excitation filter of 485 nm and an emission filter of 530 nm. Dissociation constants were determined with Prism (GraphPad Software Inc) using a one-site total binding model.

### Competitive and Non-Competitive Phage ELISA

Competitive and non-competitive phage ELISAs were performed as previously described with minor modifications to the protocol. Peptide sequences used for these analyses can be found in Table 4.

Streptavidin (50 ng/well in TBS (50 mM Tris-HCl pH 8.0, 150 mM NaCl) was coated onto MAXISORP plates (Thermo Scientific). Phage binding was detected using HRP conjugated anti-M13 secondary antibody (1/2000 dilution, GE Healthcare) in TBS buffer containing 0.05% Tween-20 (TBT buffer). Phage binding was detected using the 3,3′,5,5′-tetramethylbenzidine (TMB) (Thermo Fisher Scientific) chromogenic substrate. Plates were read using a PowerWave XS microplate reader (BioTek).

Competitive phage-ELISAs were used to determine binding affinity of SH2 superbinders for their cognate target. ELISAs were performed as previously described, biotinylated and phosphorylated peptides were immobilized on plates at the concentration of 0.2 μM, and available binding sites on streptavidin were blocked by adding 25 μl/well of 50 μM D-biotin in TBS. Following to streptavidin blocking, phage-displayed SH2 superbinders were allowed to bind to the immobilized pTyr-peptide for 20 min at 25° C. in the presence of varying concentrations (8,000-2 nM) of biotinylated and phosphorylated peptides. Data were plotted using Prism 6 software (GraphPad Software Inc) and affinity of SH2 domains was inferred from determination of the half maximal inhibition concentration (IC₅₀). IC₅₀ curves were fitted using the one-site binding Hill model and determined as the concentration of pTyr-peptide competitor that reduced by 50% binding of phage-displayed SH2 domains to plate-immobilized phosphopeptides.

### Protein Expression and Purification

SH2 domains were fused at the N-terminus to a hexa-histidine tag (SEQ ID NO: 255) and an AviTag for enzymatic biotinylation. SH2 domains fused to AviTag were transformed into Escherichia coli BL21(DE3) cells that had been previously transformed with a plasmid encoding the BirA biotin-ligase enzyme. Cells were then grown in 2YT medium containing 0.1 mg/ml carbenicillin and 34 μg/ml chloramphenicol at 37° C. with 200 rpm shaking. D-biotin (Biobasic) was added to the cultures at a final concentration of 50 μM at OD₆₀₀ 0.4, and cultures were again incubated at 37° C. with 200 rpm shaking until OD₆₀₀ 0.6. SH2 domains expression was then induced by adding 1 mM IPTG and incubating cultures at 18° C. for 18 h with 200 rpm shaking. SH2 domains were purified using Ni-NTA resin using standard methods on Ni-NTA (Qiagen). Protein purity was verified by SDS-PAGE, protein biotinylation was determined using a band-shift assay as previously described and protein concentration was determined by BCA assay (Thermo Fisher Scientific).

### Cell Culture

HEK293T, HeLa cells (human cervical cancer cell line), and K562 (bone marrow derived, chronic myelogenous leukemia cell line) were from American Type Culture Collection. Cell lines were grown at 37° C., 5% CO₂ in Dulbecco's Modified Eagle Medium (DMEM) (Life Technologies), Eagle's Minimum Essential Medium (EMEM), and Iscove's Modified Dulbecco's Medium (IMDM) respectively, containing 10% vol/vol FBS (Sigma-Aldrich), 50 U/mL penicillin and 50 μg/mL streptomycin (Sigma-Aldrich).

To stimulate protein phosphorylation and inhibit phosphotyrosyl phosphatases cells were treated with a mixture of 50 ng/ml epidermal growth factor (EGF, Sigma Millipore), 1 μg/ml insulin-like growth factor (IGF, Sigma Millipore) and 1 mM sodium orthovanadate (Sigma), incubated at 37° C. with 5% CO₂ for 20 minutes. Following incubation, media was discarded, cells were washed three times with ice cold PBS, harvested and flash frozen in liquid nitrogen. To maximize the diversity of phosphoproteins in cell lysates untreated cells at about 85% confluence were washed and harvested as described above.

### Western Blotting

To assess protein phosphorylation, following separation via SDS-PAGE, samples were analyzed by Western Blot. Membranes were blocked at 25° C. for 1 h with gentle shaking in 50 mM Tris-HCl pH 7.5, 150 mM NaCl, 5% (w/v) BSA (Blocking buffer) and probed with 0.5 μg/ml anti-phosphotyrosine-HRP conjugated antibody (Pierce) in Blocking buffer containing 0.05% Tween-20 for 1 h at 25° C. with gentle shaking.

Membranes were washed three times with washing buffer (50 mM Tris-HCl pH 7.5, 150 mM NaCl, 0.5% Tween-20) for 10 min at 25° C. with gentle shaking, and phosphorylated proteins were detected with the ECL Western Blotting Substrate (ThermoFisher).

### Preparation of Total Cell Protein Extracts and Trypsin Digestion

Cells lines harvested previously were re-suspended in lysis buffer (6M Urea, Tris-HCl pH 8.0, 150 mM NaCl, EDTA-free Protease Inhibitor Cocktail (Millipore Sigma), 1 mM Sodium Orthovanadate, 10 mM PMSF, 1 mM NaF, 10 mM Sodium Pyrophosphate, 1 mM Glycerophosphate) and sonicated 3 times for 10 seconds at 30% amplitude on ice. Cell lysates were centrifuged at 12,000 rpm at 4° C., and supernatants were collected. Cysteine residues were reduced by adding dithiothreitol (DTT, Sigma-Aldrich) to a final concentration of 5 mM and incubating samples at 56° C. for 25 min. Following reduction, cysteine residues were alkylated by incubating samples with 14 mM chloroacetamide in the dark at 25° C. for 30 min. The reaction was quenched by adding DTT to a final concentration of 10 mM and incubating mixtures for 15 min at 25° C.

Proteins were purified using the chloroform methanol extraction method. Briefly, 100 μl of 1 μg/μl protein in lysis buffer were added to 400 μl methanol and vortexed to ensure mixing. Following methanol addition, 100 μl chloroform were added to samples and mixed using a vortex. Finally, 300 μl USP sterile water (Nurse Assist) were added to the mixture and vortexed shortly. Samples were centrifuged for 1 min at 12,000 rpm revealing two-phase separated layers with the interface between the two layers containing purified proteins. Following removal of the top layer, 400 μl methanol were added, samples were mixed by using a vortex and centrifuged at 14,000 rpm for 10 minutes to pellet proteins. Supernatant was removed and the protein pellet was air-dried for 30 min at room temperature. Proteins were then resuspended in 100 μl of 50 mM ammonium bicarbonate, samples concentration was determined via BCA assay (Thermo Scientific) and proteins were digested overnight at 37° C. with gentle shaking using N-tosyl-L-phenylalanine chloromethyl ketone (TPCK)-treated trypsin (Sigma-Aldrich) with a trypsin:protein ratio of 1:50 (w/w). Following overnight incubation, samples were further digested by adding trypsin to samples as previously described and incubating lysates at 37° C. for 3 hours with gentle shaking. Protein digestion was confirmed by SDS-PAGE.

Phosphorylated serine, threonine, and tyrosine (pSer, pThr, pTyr)-containing peptides were enriched via using the High-Select™ Fe-NTA phosphopeptide Enrichment Kit (ThermoFisher Scientific) according to the manufacturer's instructions. In addition, pTyr-peptides were enriched using protein A Sepharose resin (Abcam) containing an anti-phosphotyrosine antibody (4G10; Millipore Sigma). Resin was incubated with the peptide mixtures for 3 hours at 4° C. with end-over-end rotation. The resin was washed 3 times with 1 ml of a 50 mM Hepes pH 7.5, 150 mM NaCl solution (HBS buffer), and pTyr-peptides were eluted using 100 mM phenyl phosphate (Sigma-Aldrich). Peptides purified from HeLa, HEK293T, and K562 cell lysates using iron immobilized metal ion affinity chromatography (Fe-IMAC) and an anti-phosphotyrosine antibody were combined and lyophilized. Lyophilized samples were resuspended TBS (pH=8.0) and their concentration was determined using BCA assay (ThermoFisher).

Purified phosphopeptides were divided into 11 identical pools and labelled with a unique amine-reactive isobaric tandem mass tag (TMT) using the TMT10plex Isobaric Label Reagent Set and the TMT11-131C Label Reagent (Thermo Fisher Scientific™). Labelling reactions were performed according to the manufacturer's instructions.

### Pull-Down Tests of TMT-Labelled Phosphopeptides

Pull-down tests were performed by immobilizing biotinylated SH2 superbinders onto Magne™ Streptavidin Beads (Promega). 50 μL of high-capacity beads were washed with 1 ml of Tris-HCl pH 7.5, 150 NaCl (TBS buffer) for 3 times, before incubation for 1 hour at 4° C. of the beads with biotinylated Avi-tagged SH2 domains. Following immobilization of SH2 domains on beads, excess of SH2 domains was removed, beads were washed as previously described and then resuspended in 95 μL of TBS buffer. 5 μl of TMT-labeled phosphorylated peptides containing 1.67 μg peptide (1.51×10⁻⁹ M) were added to the SH2 domain-immobilized beads and allowed to incubate for 3 hours at 4° C. with gentle nutation. Following phosphopeptide binding, beads were washed as before to remove unbound peptides. Bound peptides were eluted twice using 40 μl of 60% Acetonitrile solution containing 0.1% trifluoroacetic acid (TFA) (vol/vol). Following elution, TMT-labelled phosphopeptides eluted from different beads were combined, lyophilized, and desalted using C18 Ziptips (Sigma) according to manufacturer's instructions and stored at −80° C.

To determine the binding specificity of SH2 domain superbinders, pull-down tests were performed by capturing on magnetic beads different amounts (4.8, 24, or 120 μg) of SH2 domains and adding to beads a constant concentration of TMT-labelled phosphopeptides. As a positive control, saturating concentrations of biotinylated Avi-tagged superSrc beads were immobilized on beads, whereas beads decorated with biotinylated Avi-tagged GST were used as negative control.

### LC-MS and Mass Spectra Analysis

Enriched TMT-labelled phosphopeptides were separated on a 50-cm Easy-Spray column (75-μm inner diameter) packed with 2 μm C18 resin (Thermo Scientific, Odense, Denmark) and heated to 50° C. The column is connected to an EASY nLC 1000 chromatography system (Thermo-Fisher Scientific) feeding into a nano-ESI source, coupled to an Orbitrap Fusion™ Lumos™ Tribrid™ mass spectrometer (Thermo Fisher Scientific). Phosphopeptides were eluted during at a flow-rate of 250 nl/min over 120 min using a 0-40% acetonitrile gradient. Eluted peptides were injected into the mass spectrometer, and data were acquired at a 70,000 resolution with a m/z 400. Mass spectra were acquired in full scan mode with HCD (high-energy collision dissociation) fragmentation. Acquired data were analyzed by MaxQuant software (version 1.6.3.4) for identification and quantification on the Human Proteome (Swiss-Prot database, 2019 version, protein entries: 74,349). Group-specific parameters were set to reporter ion MS3 and 11-plex (TMTllplex Lys/Nter 126C, 127C/N, 128C/N, 129C/N, 130C/N, 131C/N reporter ions) with a reporter mass tolerance of 0.003. Search parameters include cysteine carbamidomethylation as a fixed modification, N-terminal acetylation, methionine oxidation, and pSer, pThr and pTyr as variable modifications. For statistical evaluation, posterior error probability (PEP) and false discovery rate (FDR) were used, applying the default PEP value in MaxQuant and a 0.01 FDR. In the analysis, two mis-cleavage events are allowed, and a minimum of seven amino acids per identified peptide were required. Peptide identification was based on a search with an initial mass deviation of the precursor ion of up to 6 ppm, and the allowed fragment mass deviation was set to 20 ppm. To match peptide identity across different replicates, “match between runs” option in MaxQuant was enabled with a time window of 2 min.

### Data Analysis

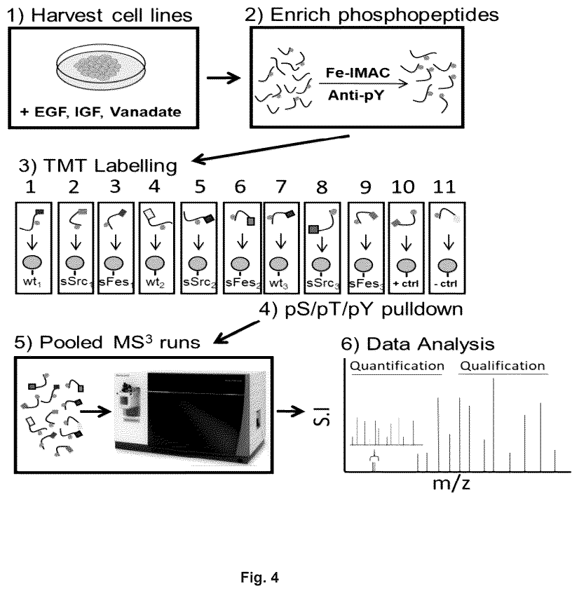

ELISA data were analyzed using Prism 6 software (GraphPad Software Inc). Mass spectrometry data was analyzed using custom scripts written in Python (v 3.7.2) using the Matplotlib (v 3.1.1) package for statistical inference and data visualization. Structural alignments and analysis were also performed using custom scripts written in Python (v 3.7.2) and visualized in PyMol (v 2.3.2). Schematic workflow for TMT-based quantitative proteomics analyses is illustrated in FIG. 4.

The sequences of all human SH2 domains were downloaded from the Universal Protein Resource (UniProt; https://www.uniprot.org/) and aligned them using the Constraint Based Alignment Tool (COBALT)30 from NCBI (National Center for Biotechnology Information; https://www.ncbi.nlm.nih.gov/tools/cobalt/re_cobalt.cgi). The sequence alignment was visualized using Jalview software (https://www.jalview.org/). Highlighted using grey boxes and bolded text in the alignment is the αA-2 (position 33), BC Loop (positions 59-68), βC-2 (position 70) and βD-6 (position 102) that correspond to residues used to make SH2 superbinders using our grafting technique. Note that dashes (“-”) depict gaps in the sequence alignment.

### Example 2 Development of High Affinity Variants of the Fes Sh2 Domain

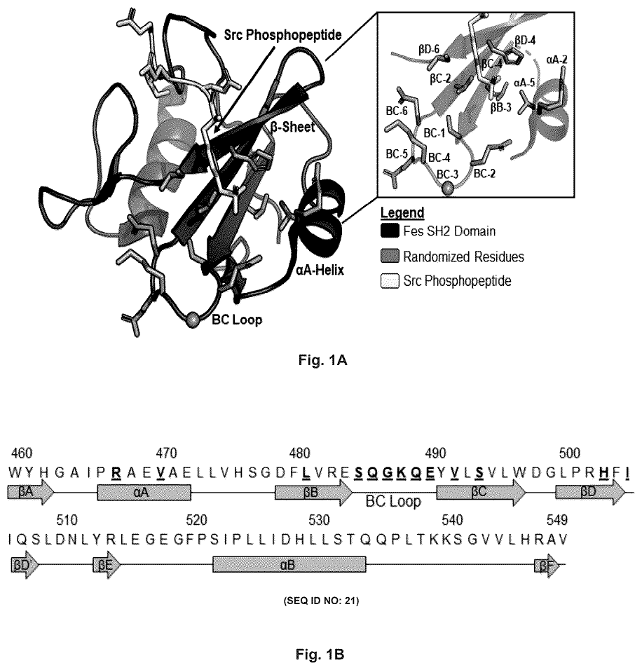

To further expand the range of ligand specificities that could be targeted with SH2 superbinders, Fes-SH2 is chosen to be modified. Fes-SH2 shares only 35% sequence identity with Src-SH2. To aid library design, the structure of Fes-SH2 was examined, as there is no structure of ligand-bound Fes-SH2 currently available. The structure of Fes-SH2 was superposed with the structure of Src-SH2 in complex with a peptide ligand ({pTyr}EEIE (SEQ ID NO: 256)) (FIG. 1A). Fes-SH2 residues that were in analogous positions with those selected for randomization in the Src-SH2 phage display library were then identified. The residues selected were those oriented towards the ligand pTyr residue and had at least one atom in the side chain within 10 Å of any atom of the pTyr residue. Applying these criteria, a set of 13 residues was chosen for diversification, including two residues in the αA-helix, five residues in the β-sheet, and all six residues comprising the BC-loop (FIG. 1B). A phage-displayed library was constructed with 1.6×10¹⁰ unique variants, using a soft randomization strategy that favored the wild-type (wt) sequence but allowed for diversity across all 13 positions.

Phage pools representing the library were cycled through five rounds of selections for binding to an immobilized pTyr-containing peptide (pEZ) derived from the natural Fes-SH2 ligand Ezrin (sequence: PPV{pTyr}EPVS (SEQ ID NO: 257)). Phage enzyme-linked immunosorbent assays (ELISAs) were used to identify individual clones that exhibited binding signals for the peptide pEZ that were at least ten-fold higher than those for an unphosphorylated peptide (EZ) with the same primary sequence, and DNA sequencing revealed six unique Fes-SH2 variants, which were named superFes-SH2-1-6 (sFes¹⁻⁶, Table 3; Table 1 SEQ IDs: 130-135). The variants were purified as free proteins and fluorescence polarization binding assays with peptide pEZ revealed that, compared with the wt protein (IC₅₀=1.3 μM), the variants exhibited 28-490-fold enhancements in apparent affinities (IC₅₀=2.7-48 nM).

TABLE 3
Unique clones isolated after five rounds of panning of the Fes SH2 library
against the ezrin phosphopeptide (PPV{pTyr}EPV (SEQ ID NO: 258))

| | | | | | | | | | | | | | | IC₅₀ | Fold | |
|---|---|---|---|---|---|---|---|---|---|---|---|---|---|---|---|---|
| | αA-2 | αA-5 | βB-3 | BC-1 | BC-2 | BC-3 | BC-4 | BC-5 | BC-6 | βC-2 | βC-4 | βD-4 | βD-6 | (μM) | Change | SEQ |
| wt | R | V | L | S | Q | G | K | Q | E | V | S | H | I | 1.3 | 1 | ID |
| sFes¹ | — | — | — | G | — | S | Q | P | D | — | — | — | — | 0.0027 | 493.20 | 130 |
| sFes² | — | — | — | R | — | R | — | — | — | — | — | — | — | 0.0039 | 344.10 | 131 |
| sFes³ | — | — | — | — | P | R | I | — | — | — | — | — | — | 0.0140 | 95.86 | 132 |
| sFes⁴ | — | — | — | — | — | — | R | K | V | — | — | — | — | 0.0174 | 76.95 | 133 |
| sFes⁵ | — | — | — | — | I | S | — | — | G | — | — | — | L | 0.0397 | 33.80 | 134 |
| sFes⁶ | — | — | I | — | — | T | Y | P | G | — | — | — | — | 0.0480 | 27.96 | 135 |

The IC₅₀ of each unique clone listed in Table 3 was determined using fluorescence polarization against the ezrin phosphopeptide. “wt” is the unmodified Fes SH2.

### Example 3—Src and Fes Superbinder Motif Swapping

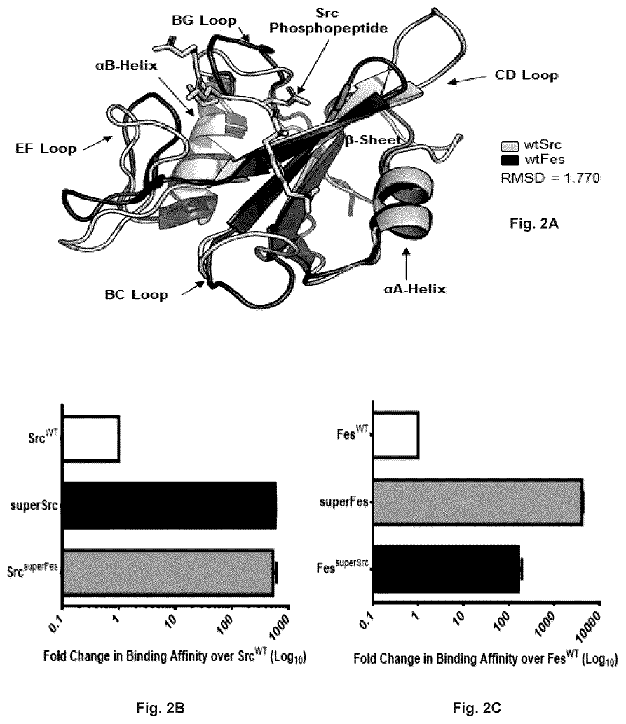

Structural-functional analysis was performed to provided basis for grafting the superbinding motifs from sSrc and sFes into one another to increase binding affinity. The structures of Src (PDB ID: 1HCT) and Fes (PDB ID: 1WQU) were superimposed and the superimposition shows that despite having different primary sequences the core structure of the domain is quite conserved (FIG. 2A).

Therefore, key residues involved in binding the pTyr residues were identified and the BC loop and corresponding backside residues were grafted from sSrc into the Fes SH2 domain and vice versa and assayed for binding affinity (IC50) using competitive phage ELISA (Table 4). The results showed that grafting the sSrc motif into Fes or the sFes motif into Src could enhance the affinity of the domain 650 and 125 times, respectively (FIGS. 2B-2C).

TABLE 4
Superbinder motif swapping between the Src and Fes SH2 domains

| | αA-2 | BC-1 | BC-2 | BC-3 | BC-4 | BC-5 | BC-6 | BC-7 | βC-2 | βD-6 | SEQ ID |
|---|---|---|---|---|---|---|---|---|---|---|---|
| Src^{wt} | R | S | E | T | T | K | G | A | S | K | 99 |
| superSrc | — | — | — | — | V | — | — | — | A | L | 136 |
| Src^{sFes} | — | G | Q | S | Q | P | D | | V | I | 167 |
| Fes^{wt} | R | S | Q | G | K | Q | E | | V | I | 21 |
| superFes | — | G | — | S | Q | P | D | | — | — | 130 |
| Fes^{sSrc} | — | S | E | T | V | K | G | A | A | L | 145 |

The IC50 of each unique clone listed in Table 4 was determined using competitive phage ELISA against the pMT peptide (EEPQ{pTyr}EEIPIY (SEQ ID NO: 181)) for Src variants and the Ezrin peptide (PPV{pTyr}EPVS (SEQ ID NO: 257)) for Fes variants and is shown in FIGS. 2B-C. “wt” is the unmodified Src or Fes SH2.

### Example 5. Motif Grafting into a Diverse Set of Sh2 Domains

Additional SH2 domains were then selected to test the grafting strategy based on: (i) favourable expression profiles in bacterial systems (>1 mg/L bacterial expression), (ii) having a known structure, (iii) known peptide specificity profile, and (iv) having varying degrees of sequence relatedness to both Src and Fes SH2 domains (30-90% primary amino sequence homology). To aid in the selection, the amino acid sequences of all human SH2 domains from the Universal Protein Resource (UniProt; https://www.uniprot.org/) were downloaded and aligned using the Constraint Based Alignment Tool (COBALT) from NCBI (National Center for Biotechnology Information; https://www.ncbi.nlm.nih.gov/tools/cobalt/re_cobalt.cgi). The positions of the residues essential for binding the pTyr residue in both Src and Fes SH2 domains in all other human SH2 domains using this alignment were identified (Table 1).

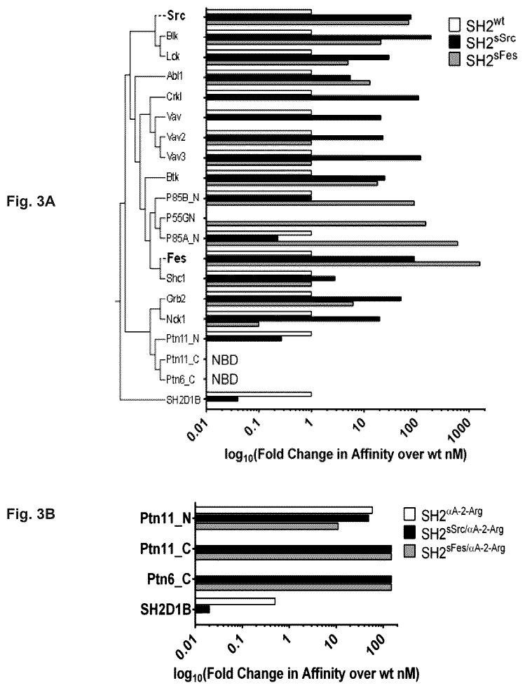

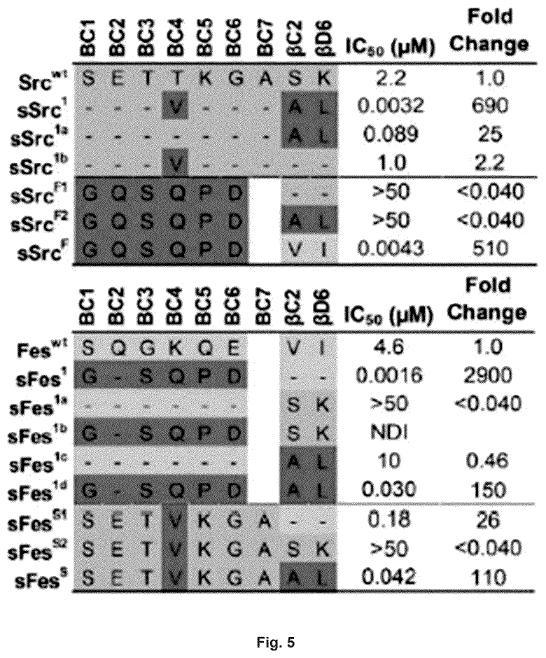

From this analysis 18 suitable SH2 domains were identified. Both the sSrc and sFes superbinder motifs were grafted into those SH2 domains. Proteins from each domain were expressed and purified as wt, sSrc, and sFes versions and their IC₅₀ values (FIG. 3A) against a phosphopeptide were determined (Table 4, FIG. 5). The affinities of the wt SH2 domains ranged from 540 to >20,000 nM for their respective phosphopeptides which is consistent with the affinities reported in the literature. For the grafted SH2 domains, the sSrc or sFes graft was sufficient to increase affinity from 2.8 to 610-fold.

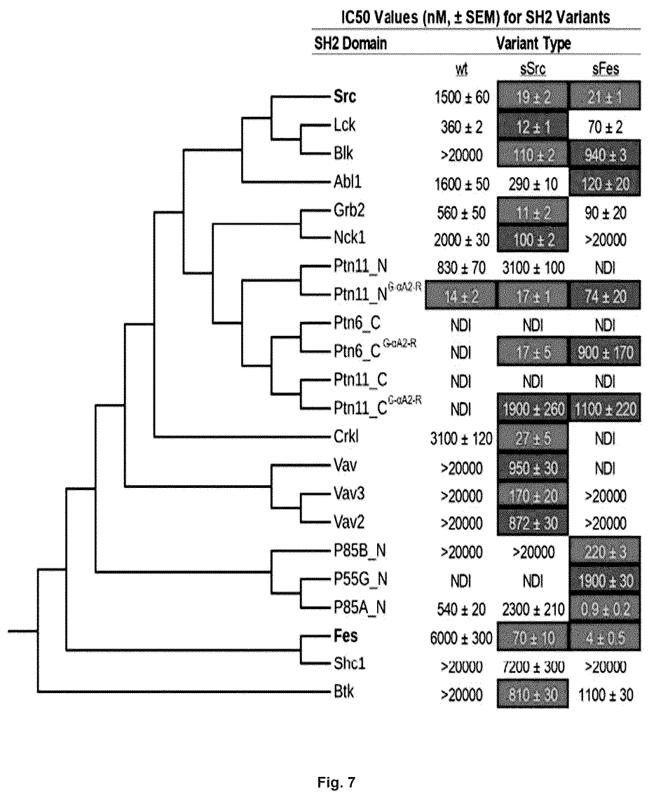

There was, however, a subset of SH2 domains (Ptn11_N, Ptn11_C, Ptn6_C, and SH2D1B) that showed no improvement in affinity with either the sSrc or sFes graft. Their sequences (Table 1) were analyzed further and the results show that these domains have a glycine or lysine residue in the crucial αA-2 position in lieu of Arg. Structural comparison of the sequences show there are interactions between the pTyr moiety and Arg-αA2 (FIGS. 6A-6D). This position was then mutated to Arg and again assayed for binding affinity by competitive ELISA to determine their IC50 values (FIG. 3B). This single point mutation substantially increased binding from 830 nM in Ptn11_N^{wt} to 14 nM in Ptn11_N^{αA-2-Arg} (FIG. 7). The αA-2-Arg mutation was not sufficient to enhance the binding of both Ptn11_C^{wt} and Ptn6_C^{wt}, but when combined with the superbinder motifs from sFes and sSrc, the binding affinity increased to 1100 and 17 nM in the Ptn11_C^{sFes/αA-2-Arg} and Ptn6_C^{ssre/αA-2-Arg} mutants, respectively (FIG. 7). The affinity could not be improved further in SH2D1B using the superbinding grafts or the αA-2-Arg substitution.

TABLE 5
Peptides used to determine the affinity
of SH2 domain variants.

| SEQ ID | | |
|---|---|---|
| NO | Domain | Peptide Used |
| 181 | Src | EEPQ{pTyr}EEIPIY |
| 182 | Blk | KQVE{pTyr}LDLDLD |
| 183 | Lck | EEPQ{pTyr}EEIPIY |
| 184 | Ab11 | SSLS{pTyr}TNPAVA |
| 185 | Crkl | EEHV{pTyr}SFPNKQ |
| 186 | Vav | PPPV{pTyr}EPVSYH |
| 187 | Vav2 | PPPV{pTyr}EPVSYH |
| 188 | Vav3 | PPPV{pTyr}EPVSYH |
| 189 | Btk | EEPQ{pTyr}EEIPIY |
| 190 | P85B_N | DDPS{pTyr}VNVQNL |
| 191 | P55G_N | DDPS{pTyr}VNVQNL |
| 192 | P85A_N | STNE{pTyr}MDMKPG |
| 193 | Fes | PPPV{pTyr}EPVSYH |
| 194 | SHC1 | EEPQ{pTyr}EEIPIY |
| 195 | Grb2 | DDPS{pTyr}VNVQNL |
| 196 | Nck1 | PPPT{pTyr}TEVDP |
| 197 | Ptn11_N | KQVE{pTyr}LDLDLD |
| 198 | Ptn11_C | PPPT{pTyr}TEVDP |
| 199 | Ptn6_C | QGVI{pTyr}SDLNLP |
| 200 | SH2D1B | SLTI{pTyr}AQVQKA |
| 201 | Ptn11_N^{G12R} | KQVE{pTyr}LDLDLD |
| 202 | Ptn11_C^{G12R} | PPPT{pTyr}TEVDP |
| 203 | Ptn6_C1^{G12R} | QGVI{pTyr}SDLNLP |

Peptides listed in Table 5 were used to assay for binding affinity of each corresponding SH2 domain. Affinities for these interactions are shown in FIGS. 3A and 3B.

### Example 6—Superbinder Sh2 Domains as Affinity Pull-Down Tools for Mass Spectromety

To examine further whether SH2 domain superbinders retain their intrinsic binding specificity, pTyr-peptide pulldown was performed using the wt and both the superFes and superSrc versions of a subset of SH2 domains. To maximize the diversity and concentration of phosphorylated targets, pTyr-peptides (Table 6; SEQ ID NOS: 204-253, y in lower case indicates phosphorylated Tyrosine) were isolated from untreated and growth factor-stimulated cells (HeLa, HEK293T and K562) in the presence of an inhibitor of phosphotyrosyl phosphatases. Following cell lysis, cell lysates and enriched phosphorylated peptides were combined by affinity chromatography using iron immobilized metal ion affinity chromatography (Fe-IMAC) and an anti-phosphotyrosine antibody. To compare the pTyr-peptide enrichment profiles between wild type and superbinder versions of a particular SH2 domain as well as among different SH2 domain variants, the isolated phosphopeptides were divided into eleven identical peptide pools and labelled them with a unique tandem mass tag (TMT). TMT-labelled peptides were then captured with different SH2 domain variants and subjected to analysis using mass spectrometry. The results showed that SH2 superbinders can enrich unique subsets of phosphopeptides (Table 7). This result enables the use of each SH2 domain superbinder of the present disclosure to enrich unique subsets of the phosphoproteome.

TABLE 6
pTyr-peptides Sequences

| SEQ ID | |
|---|---|
| NO | pTyr-peptides Sequences |
| 204 | IGEGTyGVVYK |
| 205 | IYQyIQSR |
| 206 | GEPNVSyICSR |
| 207 | HPDIyAVPIK |
| 208 | REEPEALyAAVNK |
| 209 | TLEPVKPPTVPNDyMTSPAR |
| 210 | LIEDNEyTAR |
| 211 | IKPSSSANAIySLAAR |
| 212 | RyLENVK |
| 213 | ySPSQNSPIHHIPSR |
| 214 | yRVCNVTRR |
| 215 | GSPHYFSPFRPy |
| 216 | SESVVyADIR |
| 217 | SLPSGSHQGPVIyAQLDHSGGHHSDK |
| 218 | AVDGyVKPQIK |
| 219 | LGHyATQLQK |
| 220 | LMTGDTyTAHAGAK |
| 221 | IADPEHDHTGFLTEyVATR |
| 222 | VADPDHDHTGFLTEyVATR |
| 223 | GIVVyTGDR |
| 224 | HySVVLPTVSHSGFLYK |
| 225 | TLSEVDyAPAGPAR |
| 226 | QyDEKLAQK |
| 227 | NSLETLLyKPVDR |
| 228 | ANGTTyATYER |
| 229 | ANGTTYATyER |
| 230 | MSLTAlyDK |
| 231 | FLNAESyYK |
| 232 | yRSDIHTEAVQAALAK |
| 233 | MAVILGIFGTVQyRSR |
| 234 | LCDFGSASHVADNDITPyLVSR |
| 235 | ANyQTLK |
| 236 | HTDDEMTGyVATR |
| 237 | YSRLSSTDDGyIDLQFK |
| 238 | yKLTVGKYR |
| 239 | GNFQDyVRQAVSIARQVPGTPVK |
| 240 | GHGQPGADAEKPFyVNVEFHHER |
| 241 | IEKyNVPLNR |
| 242 | AySDLSR |
| 243 | VQIyHNPTANSFR |
| 244 | LRSLyANYK |
| 245 | VIEDNEyTAR |
| 246 | GLPSDyGR |
| 247 | yVDMSVKTK |
| 248 | PGQATIFVyLK |
| 249 | VDVNSVyQK |
| 250 | NEVyDNYQNWTSLK |
| 251 | SHGyMSTPPPVK |
| 252 | VyISAVLRDQR |
| 253 | NHTYPHLGyHHPMR |

TABLE 7
Peptide enrichment profiles by a select subset of SH2 superbinders analyzed
using mass spectrometry

| Src | Fes | Grb2 | P85A_N | PTN11_C |
|---|---|---|---|---|
| IGEGTyGVVYK | IGEGTyGVVYK | IGEGTyGVVYK | IGEGTyGVVYK | IGEGTyGVVYK |
| (SEQ ID NO: 204) | (SEQ ID NO: 204) | (SEQ ID NO: 204) | (SEQ ID NO: | (SEQ ID NO: 204) |
| | | | 204) | |
| IYQyIQSR | LCDFGSASHVA | yRVCNVTRR | IYQyIQSR | HPDIyAVPIK |
| (SEQ ID NO: 205) | DNDITPyLVSR | (SEQ ID NO: 214) | (SEQ ID NO: | (SEQ ID NO: 207) |
| | (SEQ ID NO: 234) | | 205) | |
| GEPNVSyICSR | IYQyIQSR | GHGQPGADAEK | VQIyHNPTANSF | LIEDNEyTAR |
| (SEQ ID NO: 206) | (SEQ ID NO: 205) | PFyVNVEFHHER | R | (SEQ ID NO: 210) |
| | | (SEQ ID NO: 240) | (SEQ ID NO: 243 | |
| HPDIyAVPIK | HPDIyAVPIK | LMTGDTyTAHA | HTDDEMTGyV | RyLENVK |
| (SEQ ID NO: 207) | (SEQ ID NO: 207) | GAK | ATR | (SEQ ID NO: 212) |
| | | (SEQ ID NO: 220) | (SEQ ID NO: | |
| | | | 236) | |
| REEPEALyAAVNK | ANyQTLK | IADPEHDHTGFL | RyLENVK | PGQATIFVyLK |
| (SEQ ID NO: 208) | (SEQ ID NO: 235) | TEyVATR | (SEQ ID NO: | (SEQ ID NO: 248) |
| | | (SEQ ID NO: 221) | 212) | |
| TLEPVKPPTVPNDy | HTDDEMTGyVA | VADPDHDHTGFL | LRSLyANYK | VDVNSVyQK |
| MTSPAR | TR | TEyVATR | (SEQ ID NO: | (SEQ ID NO: 249) |
| (SEQ ID NO: 209) | (SEQ ID NO: 236) | (SEQ ID NO: 222) | 244) | |
| LIEDNEyTAR | YSRLSSTDDGyI | IEKyNVPLNR | VIEDNEyTAR | SLPSGSHQGPVIy |
| (SEQ ID NO: 210) | DLQFK | (SEQ ID NO: 241) | (SEQ ID NO: | AQLDHSGGHHS |
| | (SEQ ID NO: 237) | | 245) | DK |
| | | | | (SEQ ID NO: 217) |
| IKPSSSANAIySLAA | yKLTVGKYR | AySDLSR | AVDGyVKPQIK | IADPEHDHTGFL |
| R | (SEQ ID NO: | (SEQ ID NO: 242) | (SEQ ID NO: | TEyVATR |
| (SEQ ID NO: 211) | 238) | | 218) | (SEQ ID NO: 221) |
| RyLENVK | RyLENVK | IGEGTyGVVYK | IADPEHDHTGF | GIVVyTGDR |
| (SEQ ID NO: 212) | (SEQ ID NO: 212) | (SEQ ID NO: 204) | LTEyVATR | (SEQ ID NO: 223) |
| | | | (SEQ ID NO: | |
| | | | 221) | |
| ySPSQNSPIHHIPSR | GNFQDyVRQAV | | VADPDHDHTG | NEVyDNYQNWT |
| (SEQ ID NO: 213) | SIARQVPGTPVK | | FLTEyVATR | SLK (SEQ ID NO: |
| | (SEQ ID NO: 239) | | (SEQ ID NO: | 250) |
| | | | 222) | |
| yRVCNVTRR | SESVVyADIR | | GIVVyTGDR | SHGyMSTPPPVK |
| (SEQ ID NO: 214) | (SEQ ID NO: 216) | | (SEQ ID NO: | (SEQ ID NO: 251) |
| | | | 223) | |
| GSPHYFSPFRPy | GHGQPGADAEK | | GLPSDyGR | VyISAVLRDQR |
| (SEQ ID NO: 215) | PFyVNVEFHHER | | (SEQ ID NO: | (SEQ ID NO: 252) |
| | (SEQ ID NO: 240) | | 246) | |
| SESVVyADIR | LMTGDTyTAHA | | TLSEVDyAPAG | NHTYPHLGyHHP |
| (SEQ ID NO: 216) | GAK | | PAR | MR |
| | (SEQ ID NO: 220) | | (SEQ ID NO: | (SEQ ID NO: 253) |
| | | | 225) | |
| SLPSGSHQGPVIyAQ | IADPEHDHTGFL | | yVDMSVKTK | yRSDIHTEAVQA |
| LDHSGGHHSDK | TEyVATR | | (SEQ ID NO: | ALAK |
| (SEQ ID NO: 217) | (SEQ ID NO: 221) | | 247) | (SEQ ID NO: 232) |
| AVDGyVKPQIK | VADPDHDHTGF | | | |
| (SEQ ID NO: 218) | LTEyVATR | | | |
| | (SEQ ID NO: 222) | | | |
| LGHyATQLQK | NSLETLLyKPVD | | | |
| (SEQ ID NO: 219) | R | | | |
| | (SEQ ID NO: 227) | | | |
| LMTGDTyTAHAGA | | | | |
| K | | | | |
| (SEQ ID NO: 220) | | | | |
| IADPEHDHTGFLTEy | | | | |
| VATR | | | | |
| (SEQ ID NO: 221) | | | | |
| VADPDHDHTGFLTE | | | | |
| yVATR | | | | |
| (SEQ ID NO: 222) | | | | |
| GIVVyTGDR | | | | |
| (SEQ ID NO: 223) | | | | |
| HySVVLPTVSHSGF | | | | |
| LYK | | | | |
| (SEQ ID NO: 224) | | | | |
| TLSEVDyAPAGPAR | | | | |
| (SEQ ID NO: 225) | | | | |
| QyDEKLAQK | | | | |
| (SEQ ID NO: 226) | | | | |
| NSLETLLyKPVDR | | | | |
| (SEQ ID NO: 227) | | | | |
| ANGTTyATYER | | | | |
| SEQ ID NO: 228) | | | | |
| ANGTTYATyER | | | | |
| (SEQ ID NO: 229) | | | | |
| MSLTAIyDK | | | | |
| SEQ ID NO: 230) | | | | |
| FLNAESyYK | | | | |
| (SEQ ID NO: 231) | | | | |
| yRSDIHTEAVQAAL | | | | |
| AK | | | | |
| SEQ ID NO: 232) | | | | |
| MAVILGIFGTVQyR | | | | |
| SR | | | | |
| (SEQ ID NO: 233) | | | | |

SH2 domains were adhered to streptavidin-conjugated magnetic beads and used to enrich pTyr-containing peptides from cell lysate. The peptides enriched for each subset of SH2 domains (including superbinders) are shown above. Lower case “y” denotes the position of the phosphotyrosine within the peptide.

### Example 7. A Universal Method to Create Sh2 Superbinders

Using Table 2 only as a guide, an SH2 superbinder was created using the grafting technique of the present disclosure: (1) Align the amino acid sequence of the SH2 domain in question (i.e., the SH2 domain to be modified) using a relevant or suitable alignment algorithm (e.g. COBALT¹²) to multiple SH2 domain sequences, In some aspects to multiple SH2 sequences of SEQ ID NOS: 1-122 found in Table 1 (i.e., a combination of two or more of SEQ ID NOS: 1-122 or any other SH2 sequence not included in Table 1), as long as when aligned the relevant positions described herein are covered (i.e., positions of the BC loop, βD-6, βC-2, αA-2 as indicated herein), (2a) graft a BC loop of any one of sFes1-6 motifs (SEQ IDs: 123-128) into the BC loop (positions 59-68) of the SH2 domain to be modified, if the amino acid at position 70 of the SH2 domain to be modified is hydrophilic, then substitute said hydrophilic amino acid with V into βC-2 (position 70) and if the amino acid at position 102 of the SH2 domain to be modified is hydrophilic, then substitute said hydrophilic amino acid with I into βD-6 (position 102)) or (2b) graft the BC loop of the sSrc motif (SEQ ID: 129) into the BC loop (positions 59-68) of the SH2 domain to be modified, if the amino acid at position 70 of the SH2 domain to be modified is hydrophilic, then substitute said hydrophilic amino acid with A into βC-2 (position 70) and if the amino acid at position 102 of the SH2 domain to be modified is hydrophilic, then substitute said hydrophilic amino acid with L into βD-6 (position 102)) and/or (3) graft an Arg residue into the αA-2 (position 33) of the SH2 domain to be modified. The SH2 domain sequence to be modified may be any SH2 domain (i.e., any protein domain labelled as “SH2” or “Src Homology 2” or “SH2 like” or “Src Homology 2 like” that is derived from any organism (i.e., any subject of the animal kingdom, including humans). In some aspects, the SH2 domain sequence to be modified is aligned with SH2 domain sequences SEQ ID NOS: 1-122 provided in Table 1 to assess positions to be modified in the SH2 domain of interest (i.e., positions of the BC loop, βD-6, βC-2, αA-2 as indicated before). See Table 1; SEQ IDs: 130-180 for a list of the sequences of the SH2 superbinders created using this method that were used in the ELISA binding and phosphoproteomics tests.

The amino acid sequences of additional SH2 domains (SEQ ID NOS: 130-180) with either the sSrc or sFes graft housed within the domain was tagged with His6x-AviTag—TEV Cleavage Site—FLAG Tag at its N-terminus.

The foregoing description and accompanying drawings set forth a number of representatives at the present time. Various modifications, additions, and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A composition comprising a modified Src homology 2 (SH2) domain, wherein the modified SH2 domain comprises one or more amino acid substitutions in a BC loop region, and wherein the amino acid substitutions provide for increasedphosphorylated tyrosine (pTyr) binding with the modified SH2 domain compared to an unmodified SH2 domain as measured by competitive ELISA.

2. The composition of claim 1, wherein the modified SH2 domain provides for at least 10-fold increase in binding as compared to unmodified SH2 domain.

3. The composition of claim 1, wherein the modified SH2 domain further comprises one or more amino acid substitutions in a C anti-parallel β-sheet (βC) and a D anti-parallel β-sheet (BD), wherein the amino acid substitutions result in hydrophobic interactions with pTyr.

4. The composition of claim 3, wherein the one or more amino acid substitutions in the C anti-parallel β-sheet (βC) comprise a substitution of a Cys at position 70 for an Ala or a Val.

5. The composition of claim 3, wherein the one or more amino acid substitutions in the D anti-parallel β-sheet (βD) comprise a substitution of a Lys at position 102 for a Leu or Ile.

6. The composition of claim 3, wherein the one or more amino acid substitutions in the C anti-parallel β-sheet (βC) comprise a substitution of a Cys at position 70 for an Ala, and the one or more amino acid substitutions in the D anti-parallel β-sheet (βD) comprise a substitution of a Lys at position 102 for a Leu.

7. The composition of claim 3, wherein the one or more amino acid substitutions in the C anti-parallel β-sheet (βC) comprise a substitution of a Cys at position 70 for a Val, and the one or more amino acid substitutions in the D anti-parallel β-sheet (βD) comprise a substitution of a Lys at position 102 for an Ile.

8. The composition of claim 1, wherein the modified SH2 domain further comprises an Arg residue in an αA-helix at position 33.

9. The composition of claim 8, wherein in the increase in binding comprises an increased hydrogen binding with pTyr.

10. The composition of claim 1, wherein the BC loop region comprising one or more amino acid substitutions comprises an amino acid sequence selected from SEQ ID NOS: 123-129.

11. The composition of claim 1, wherein the modified SH2 domain has at least 80% sequence identity to a sequence selected from SEQ ID NOS: 130-180.

12. The composition of claim 1, wherein the modified SH2 domain has at least 95% sequence identity to a sequence selected from SEQ ID NOS: 130-180.

13. The composition of claim 1, wherein the SH2 domain comprising one or more amino acid substitutions has at least 99% sequence identity to an amino acid sequence selected from SEQ ID NOS: 130-180.

14. The composition of claim 1, wherein the modified SH2 domain is a superbinder Fes SH2 domain variant having at least 99% sequence identity to an amino acid sequence of SEQ ID NO: 130-135.

15. The composition of claim 1, wherein the modified SH2 domain is a superbinder Fes SH2 domain variant having an amino acid sequence of SEQ ID NO: 130-135.

16. The composition of claim 1, further comprising a detectable label, wherein the detectable label is a radioactive label, a fluorescent label, a biotin-based label, an electron-dense reagent, or an enzyme.

17. The composition of claim 16, wherein the fluorescent label is fluorescein, rhodamine, or Texas Red.

18. The composition of claim 16, wherein the fluorescent label comprises one or more fluorescent proteins.

19. The composition of claim 16, wherein the enzyme is alkaline phosphatase, horseradish peroxidase, or luciferase.

20. The composition of claim 16, wherein the modified SH2 domain is covalently or non-covalently coupled to a solid carrier.

21. The composition of claim 20, wherein the modified SH2 domain further comprises a biotin label.

22. A BC loop of an SH2 domain having an amino acid sequence selected from SEQ ID NOS: 123-129.

23. A pharmaceutical composition comprising the composition of claim 1, a solubilizing agent, and an excipient.

24. A method of treating a disorder associated with protein phosphorylation, comprising administering to a subject the pharmaceutical composition of claim 23.
